(19) United States
(12) Patent Application Publication
(10) Pub. No.: US 2025/0249008 A1
(43) Pub. Date: Aug. 7, 2025

(54) PHARMACEUTICAL COMPOSITIONS OF ENSIFENTRINE FOR CHRONIC OBSTRUCTIVE PULMONARY DISEASE

(71) Applicant: Verona Pharma PLC, Cardiff (GB)

(72) Inventors: Peter Lionel SPARGO, Canterbury (GB); Tara Renae RHEAULT, Cary, NC (US); Doris K. WEILERT, Lee's Summit, MO (US); Kathleen RICKARD, Durham, NC (US); Thomas BENGTSSON, Lund (SE); Kevin TURNER, Leeds (GB)

(73) Assignee: Verona Pharma PLC, Cardiff (GB)

(21) Appl. No.: 19/046,713

(22) Filed: Feb. 6, 2025

Related U.S. Application Data
(60) Provisional application No. 63/550,786, filed on Feb. 7, 2024.

(51) Int. Cl. A61K 31/519; A61K 9/00; A61K 9/10; A61K 47/02; A61K 47/26; A61P 11/08

(52) U.S. Cl. CPC A61K 31/519; A61K 9/0078; A61K 9/10; A61K 47/02; A61K 47/26; A61P 11/08

(57) ABSTRACT

Provided herein are pharmaceutical compositions and methods for treating COPD or COPD exacerbations in subjects suffering from, for example, moderate COPD, or subjects with decreased eosinophil concentrations, or are suffering from chronic bronchitis. Also provided herein are methods of increasing trough lung function in subjects suffering from moderate COPD.

### CROSS-REFERENCE

This application claims the benefit of U.S. Provisional Application No. 63/550,786, filed Feb. 7, 2024, which is incorporated herein by reference in its entirety.

### BACKGROUND

Ensifentrine (N-(2-{(2E)-9,10-dimethoxy-4-oxo-2-[(2,4,6-trimethylphenyl)imino]-6,7-dihydro-2H-pyrimido[6,1-a]isoquinolin-3(4H)-yl}ethyl)urea; also known as RPL554) is a dual PDE3/PDE4 inhibitor and is described in WO 00/58308 A1.

As a combined PDE3/PDE4 inhibitor, ensifentrine has both bronchodilatory and anti-inflammatory activity and is useful in the treatment of respiratory disorders including chronic obstructive pulmonary disease (COPD). The chemical structure of ensifentrine is shown below.

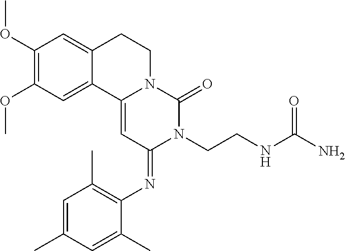 [SMILES: COc1cc2c(cc1OC)-c1c/c(=N\c3c(C)cc(C)cc3C)n(CCNC(N)=O)c(=O)n1CC2]

COPD is a progressive, long-term condition and can be experienced by patients at different levels of severity. The symptoms (e.g., breathlessness and cough) of COPD can range from mild to severe, and different treatment options can be preferred for different severities of COPD. Treatment of COPD typically comprises maintenance treatment in which a subject is administered a drug on a regular basis (e.g., once or twice daily) to improve lung function and ameliorate the symptoms of COPD.

### SUMMARY

Provided herein are methods of treating COPD, wherein the subjects being administered ensifentrine belong to one or more specific subgroups that show an increased response to ensifentrine. In some embodiments, provided herein are methods of treating COPD in subjects not having chronic bronchitis. In some embodiments, ensifentrine is effective in treating COPD in subjects without chronic bronchitis. In some embodiments, ensifentrine is effective in treating COPD in subjects with chronic bronchitis.

Also provided herein are methods of treating COPD in subjects having a reversible reversibility status of COPD. In some embodiments, ensifentrine is effective in treating COPD in subjects with reversible COPD. In some embodiments, ensifentrine is effective in treating COPD in subjects with non-reversible COPD.

Provided herein, in some embodiments, are methods of increasing trough lung function in subjects suffering from moderate COPD. In some embodiments, ensifentrine is effective in increasing trough lung function in subjects suffering from moderate COPD. In some embodiments, ensifentrine is effective in increasing trough lung function in subjects suffering from severe or very severe COPD. Also provided herein are methods of decreasing the frequency and/or severity of COPD exacerbations in subjects, such as subjects suffering from moderate COPD. In some embodiments, ensifentrine is effective in reducing the frequency/severity of COPD exacerbations in subjects with moderate COPD. In some embodiments, ensifentrine is effective in reducing the frequency/severity of COPD exacerbations in subjects with severe or very severe COPD. In some instances, also provided herein are methods of decreasing the frequency and/or severity of COPD exacerbations in subjects having blood eosinophil concentrations of less than or equal to 150 cells/μL. Ensifentrine may be uniquely suited to treat COPD and COPD exacerbations in subjects with low eosinophil concentrations, as other treatments, such as inhalable corticosteroids (ICS) lack efficacy in treating subjects with low eosinophil counts. Identification and treatment of subgroup populations that demonstrate increased responses to ensifentrine is desirable to best provide treatments for COPD and symptoms thereof.

In some embodiments, provided herein is a method of treating chronic obstructive pulmonary disease (COPD) in a human subject not having chronic bronchitis, the method comprising administering to the human subject a liquid pharmaceutical composition comprising a therapeutically effective amount of ensifentrine or a pharmaceutically acceptable salt thereof. In some embodiments, the human subject has a mean change in baseline to peak FEV₁ (forced expiratory volume in 1 second) of from about 0.1 L to about 0.2 L. In some embodiments, the human subject has a mean change in baseline to peak FEV₁ of about 0.17 L. In some embodiments, the mean change in baseline to peak FEV₁ of the human subject not having chronic bronchitis is at least 15% greater than the mean change in baseline to peak FEV₁ of a human subject who does have chronic bronchitis being administered the liquid pharmaceutical composition. In some embodiments, the mean change in baseline to peak FEV₁ of the human subject not having chronic bronchitis is at least 20% greater than the mean change in baseline to peak FEV₁ of a human subject who does have chronic bronchitis. In some embodiments, the human subject has a mean FEV₁ AUC_{0-4 h} of from about 0.1 L to about 0.2 L. In some embodiments, the human subject has a mean FEV₁ AUC_{0-4 h} of about 0.16 L. In some embodiments, the human subject has a mean FEV₁ AUC_{0-4 h} of about 0.16 L. In some embodiments, the mean FEV₁ AUC_{0-4 h} of the human subject not having chronic bronchitis is at least 20% greater than the mean FEV₁ AUC_{0-4 h} of a human subject who does have chronic bronchitis being administered the liquid pharmaceutical composition. In some embodiments, the human subject has a mean FEV₁ AUC_{0-12 h} of from about 0.1 L to about 0.2 L. In some embodiments, the human subject has a mean FEV₁ AUC_{0-12 h} of about 0.12 L. In some embodiments, the mean FEV₁ AUC_{0-12 h} of the human subject not having chronic bronchitis is at least 30% greater than the mean FEV₁ AUC_{0-12 h} of a human subject who does have chronic bronchitis being administered the liquid pharmaceutical composition. In some embodiments, the mean FEV₁ AUC_{0-12 h} of the human subject not having chronic bronchitis is at least 40% greater than the mean FEV₁ AUC_{0-12 h} of a human subject who does have chronic bronchitis being administered the liquid pharmaceutical composition. In some embodiments, the mean change in baseline to peak FEV₁ or mean FEV₁ AUC_{0-12 h} is measured after 12 weeks of administration of the liquid pharmaceutical composition. In some embodiments, provided herein is a method of increasing trough lung function in a human subject suffering from moderate chronic obstructive pulmonary disease (COPD), the method comprising administering to the human subject a liquid pharmaceutical composition comprising a therapeutically effective amount of ensifentrine or a pharmaceutically acceptable salt thereof. In some embodiments, the human subject has a change in baseline to morning trough FEV₁ of from about 30 mL to about 90 mL. In some embodiments, the human subject has a change in baseline to morning trough FEV₁ of the human subject of about 60 mL. In some embodiments, the change in baseline to morning trough FEV₁ of the human subject suffering from moderate COPD is at least 100% greater than a change in baseline to morning trough FEV₁ of a human subject suffering from severe or very severe COPD being administered the liquid pharmaceutical composition. In some embodiments, the change in baseline to morning trough FEV₁ is measured after 12 weeks of administration of the liquid pharmaceutical composition. In some embodiments, provided herein is a method of treating chronic obstructive pulmonary disease (COPD) having a reversibility status of reversible in a human subject, the method comprising administering to the human subject a liquid pharmaceutical composition comprising a therapeutically effective amount of ensifentrine or a pharmaceutically acceptable salt thereof. In some embodiments, the human subject having a reversible status of COPD has a change from baseline FEV₁ to peak FEV₁ of from about 0.1 L to about 0.2 L. In some embodiments, the human subject having a reversible status of COPD has a change from baseline FEV₁ to peak FEV₁ of about 0.17 L. In some embodiments, the change from baseline FEV₁ to peak FEV₁ for a human subject having a reversible status of COPD is at least 20% greater than a change from baseline FEV₁ to peak FEV₁ for a human subject having a non-reversible status of COPD being administered the liquid pharmaceutical composition. In some embodiments, the change from baseline FEV₁ to peak FEV₁ for a human subject having a reversible status of COPD is about 25% greater than a change from baseline FEV₁ to peak FEV₁ for a human subject having a non-reversible status of COPD being administered the liquid pharmaceutical composition. In some embodiments, the human subject having a reversible status of COPD has a FEV₁ AUC_{0-4 h} of from about 0.1 L to about 0.2 L. In some embodiments, the human subject having a reversible status of COPD has a FEV₁ AUC_{0-4 h} of about 0.16 L. In some embodiments, the FEV₁ AUC_{0-4 h} for a human subject having a reversible status of COPD is at least 25% greater than a FEV₁ AUC_{0-4 h} for a human subject having a non-reversible status of COPD being administered the liquid pharmaceutical composition. In some embodiments, the FEV₁ AUC_{0-4 h} for a human subject having a reversible status of COPD is about 33% greater than a FEV₁ AUC_{0-4 h} for a human subject having a non-reversible status of COPD being administered the liquid pharmaceutical composition. In some embodiments, provided herein is a method of decreasing the frequency and/or severity of chronic obstructive pulmonary disease (COPD) exacerbations in a human subject suffering from moderate COPD, the method comprising administering to the human subject a liquid pharmaceutical composition comprising a therapeutically effective amount of ensifentrine or a pharmaceutically acceptable salt thereof. In some embodiments, the COPD exacerbations are moderate to severe COPD exacerbations. In some embodiments, the moderate to severe COPD exacerbations are reduced by at least 30%. In some embodiments, the moderate to severe COPD exacerbations are reduced by about 36%. In some embodiments, the moderate to severe COPD exacerbations are reduced by about 44%. In some embodiments, decreasing the frequency and/or severity of COPD exacerbations comprises increasing the amount of time to a first COPD exacerbation, as measured by a hazard ratio. In some embodiments, the hazard ratio for the human subject having moderate COPD is at least 40% lower than a hazard ratio for a subject having severe or very severe COPD being administered the liquid pharmaceutical composition. In some embodiments, the hazard ratio for the human subject having moderate COPD is about 60% lower than a hazard ratio for a subject having severe or very severe COPD being administered the liquid pharmaceutical composition. In some embodiments, decreasing the frequency and/or severity of COPD exacerbations comprises decreasing exacerbation risk (e.g., as measured by time to first event). In some embodiments, provided herein is a method of decreasing the frequency and/or severity of chronic obstructive pulmonary disease (COPD) exacerbations in a human subject suffering from moderate COPD, the method comprising administering to the human subject a liquid pharmaceutical composition comprising a therapeutically effective amount of ensifentrine or a pharmaceutically acceptable salt thereof. In some embodiments, the COPD exacerbations are moderate to severe COPD exacerbations. In some embodiments, the moderate to severe COPD exacerbations are reduced by at least 30%. In some embodiments, the moderate to severe COPD exacerbations are reduced by about 36%. In some embodiments, the moderate to severe COPD exacerbations are reduced by about 44%. In some embodiments, decreasing the frequency and/or severity of COPD exacerbations comprises increasing the amount of time to a first COPD exacerbation, as measured by a hazard ratio. In some embodiments, the hazard ratio for the human subject having moderate COPD is at least 40% lower than a hazard ratio for a subject having severe or very severe COPD being administered the liquid pharmaceutical composition. In some embodiments, the hazard ratio for the human subject having moderate COPD is about 60% lower than a hazard ratio for a subject having severe or very severe COPD being administered the liquid pharmaceutical composition. In some embodiments, decreasing the frequency and/or severity of COPD exacerbations comprises decreasing exacerbation risk (e.g., as measured by time to first event). In some embodiments, the exacerbation risk is reduced by at least 40%. In some embodiments, the exacerbation risk is reduced by at least 50%. In some embodiments, the human subject comprises a baseline blood eosinophils concentration of less than or equal to 150 cells/μL. In some embodiments, an exacerbation rate ratio for the human subject having moderate COPD is at least 50% lower than an exacerbation rate for a human subject having severe or very severe COPD being administered the liquid pharmaceutical composition. In some embodiments, the exacerbation rate ratio for the human subject having moderate COPD is about 60% lower than an exacerbation rate for a human subject having severe or very severe COPD being administered the liquid pharmaceutical composition. In some embodiments, provided herein is a method of treating chronic obstructive pulmonary disease (COPD) in a human subject being administered inhalable corticosteroids (ICS) and a long acting beta agonist (LABA), the method comprising administering to the human subject a liquid pharmaceutical composition comprising a therapeutically effective amount of ensifentrine or a pharmaceutically acceptable salt thereof. In some embodiments, a change in TDI (transition dyspnea index) is at least 100% greater for the human subject being administered the liquid pharmaceutical composition than for a human subject being administered a placebo. In some embodiments, a change in TDI (transition dyspnea index) is about 125% greater for the human subject being administered the liquid pharmaceutical composition than for a human subject being administered a placebo. In some embodiments, the administering is by inhalation. In some embodiments, the inhalation is by nebulizer. In some embodiments, the ensifentrine is ensifentrine free base. In some embodiments, the administering to the human subject is at least once, twice, or three times per day. In some embodiments, the administering to the human subject is at least twice a day. In some embodiments, the therapeutically effective amount of ensifentrine or the pharmaceutically acceptable salt thereof is of from about 2 mg to about 4 mg. In some embodiments, the therapeutically effective amount of ensifentrine or the pharmaceutically acceptable salt thereof is 3 mg. In some embodiments, the therapeutically effective amount of ensifentrine or the pharmaceutically acceptable salt thereof comprises a total daily dose of the ensifentrine or the pharmaceutically acceptable salt thereof of from about 0.5 mg to about 10 mg. In some embodiments, the therapeutically effective amount of ensifentrine or the pharmaceutically acceptable salt thereof comprises a total daily dose of the ensifentrine or the pharmaceutically acceptable salt thereof of from about 5 mg to about 7 mg. In some embodiments, the administering to the human subject comprises a first dose and a second dose. In some embodiments, the first dose comprises of from about 2 mg to about 4 mg of ensifentrine or the pharmaceutically acceptable salt thereof and the second dose comprises of from about 2 mg to about 4 mg of ensifentrine or the pharmaceutically acceptable salt thereof. In some embodiments, the first and the second dose comprise about 3 mg of the ensifentrine or the pharmaceutically acceptable salt thereof. In some embodiments, the first dose is administered in the morning and the second dose is administered in the evening. In some embodiments, the first dose is administered within three hours after the human subject waking, and the second dose is administered within three hours before the human subject sleeps. In some embodiments, the first dose and the second dose are administered to the human subject about 10 h to about 14 h apart. In some embodiments, the method comprises administering the liquid pharmaceutical composition to the subject at least once per day for at least 8 weeks. In some embodiments, the administering to the human subject is at least once per day for at least 16 weeks. In some embodiments, the administering to the human subject is at least one per day for at least 24 weeks. In some embodiments, the liquid pharmaceutical composition is an inhalable pharmaceutical composition. In some embodiments, the liquid pharmaceutical composition comprises a suspension of ensifentrine particles comprising the ensifentrine or a pharmaceutically acceptable salt thereof in a diluent. In some embodiments, the ensifentrine particles comprise at least 95% by weight of the ensifentrine or the pharmaceutically acceptable salt thereof. In some embodiments, the ensifentrine particles comprise at least 95% by weight of ensifentrine free base. In some embodiments, the ensifentrine is in the form of a crystalline solid consisting of greater than 99% by weight of ensifentrine free base, at least 95% in the polymorphic form of a thermodynamically stable polymorph (I) of ensifentrine free base having the following structural parameters obtained by single crystal analysis, such as shown in Table 1.

TABLE 1

| Wavelength | 0.71073 Å | |
|---|---|---|
| Crystal system | Triclinic | |
| Space group | P-1 | |
| Unit cell dimensions | a = 8.1246(4) Å | α = 91.583(4)° |
| | b = 11.4573(5) Å | β = 90.299(4)° |
| | c = 13.2398(6) Å | γ = 99.628(4)° |
| Volume | 1214.56(10) Å³ | |
| Z | 2 | |

In some embodiments, the ensifentrine particles comprise: of from about 0% to about 0.6% by weight of 1,3-bis(2-(2-(mesitylimino)-9,10-dimethoxy-4-oxo-6,7-dihydro-2H-pyrimido[6,1-a]isoquinolin-3(4H)-yl)ethyl)urea (BMIQU) relative to the total weight of the ensifentrine or the pharmaceutically acceptable salt thereof. In some embodiments, the ensifentrine particles comprise of from about 0% to about 0.5% by weight of a biuret impurity of formula (A) relative to the total weight of the ensifentrine or the pharmaceutically acceptable salt thereof:

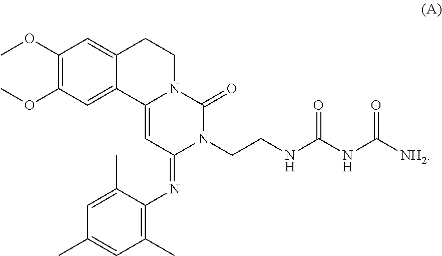 [SMILES: COc1cc2c(cc1OC)-c1c/c(=N\c3c(C)cc(C)cc3C)n(CCNC(=O)NC(N)=O)c(=O)n1CC2]

In some embodiments, the ensifentrine particles comprise of from about 0% to about 0.3% by weight of the biuret impurity relative to the total weight of the ensifentrine or the pharmaceutically acceptable salt thereof. the ensifentrine particles comprise of from about 0% to about 0.3% by weight of the biuret impurity relative to the total weight of the ensifentrine or the pharmaceutically acceptable salt thereof. In some embodiments, the ensifentrine particles comprise of from 0.01% to 0.3% by weight of BMIQU relative to the total weight of ensifentrine or a pharmaceutically acceptable salt thereof. the liquid pharmaceutical composition comprises: (a) ensifentrine particles; (b) a buffer; (c) one or more surfactants; and (d) a tonicity adjuster. In some embodiments, the liquid pharmaceutical composition comprises: (a) ensifentrine particles at a concentration of from about 1 mg/mL to about 1.4 mg/mL; (b) a buffer at a concentration of from about 1 mg/mL to about 2.0 mg/mL; (c) one or more surfactants at a total concentration of from about 0.3 mg/mL to about 0.8 mg/mL; and (d) a tonicity adjuster at a concentration of from about 5 mg/mL to about 10 mg/mL. In some embodiments, the liquid pharmaceutical composition comprises: (a) ensifentrine particles at a concentration of from about 1 mg/mL to about 1.4 mg/mL; (b) polysorbate 20 (Tween 20) at a concentration of from about 0.3 mg/mL to about 0.7 mg/mL; (c) sorbitan monolaurate (Span 20) at a concentration of from 0 mg/mL to about 0.1 mg/mL; (d) sodium dihydrogen phosphate dihydrate at a concentration of from about 0.5 mg/mL to about 1 mg/mL; (e) disodium hydrogen phosphate dihydrate at a concentration of from about 0.5 mg/mL to about 1 mg/mL; and (f) sodium chloride at a concentration of from about 5 mg/mL to about 10 mg/mL. In some embodiments, the liquid pharmaceutical composition comprises: (a) ensifentrine particles at a concentration of 1.2 mg/mL; (b) polysorbate 20 (Tween 20) at a concentration of 0.5 mg/mL; (c) sorbitan monolaurate (Span 20) at a concentration of 0.05 mg/mL; (d) sodium dihydrogen phosphate dihydrate at a concentration of 0.744 mg/mL; (e) disodium hydrogen phosphate dihydrate at a concentration of 0.853 mg/mL; and (f) sodium chloride at a concentration of 8.6 mg/mL. In some embodiments, a total weight of the ensifentrine or the pharmaceutically acceptable salt thereof in the liquid pharmaceutical composition is of from about 2.7 mg to about 3.3 mg. In some embodiments, the ensifentrine or the pharmaceutically acceptable salt thereof is used in combination with a muscarinic receptor antagonist, a beta-adrenergic receptor agonist, or an inhaled corticosteroid. Provided herein are uses of any one of the methods provided herein for treating COPD in a human subject.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings (also “Figure” and “FIG.” herein), of which:

FIG. 1 shows change from baseline rescue medication usage in subjects after 6, 12, or 24 weeks of administration with ensifentrine.

FIG. 2 shows increases in transition dyspnea index (TDI) by week for subjects administered ensifentrine and a placebo.

FIG. 3A shows change in FEV₁ as function of time since dose at day 1 of administration with the ensifentrine for subjects with a reversible status of COPD. FIG. 3B shows change in FEV₁ as function of time since dose at week 6 of administration with the ensifentrine for subjects with a reversible status of COPD. FIG. 3C shows change in FEV₁ as function of time since dose at week 12 of administration with the ensifentrine for subjects with a reversible status of COPD. FIG. 3D shows change in FEV₁ as function of time since dose at week 24 of administration with the ensifentrine for subjects with a reversible status of COPD.

FIG. 4A shows change in FEV₁ as function of time since dose at day 1 of administration with the ensifentrine for subjects with a non-reversible status of COPD. FIG. 4B shows change in FEV₁ as function of time since dose at week 6 of administration with the ensifentrine for subjects with a non-reversible status of COPD. FIG. 4C shows change in FEV₁ as function of time since dose at week 12 of administration with the ensifentrine for subjects with a non-reversible status of COPD. FIG. 4D shows change in FEV₁ as function of time since dose at week 24 of administration with the ensifentrine for subjects with a non-reversible status of COPD.

### DETAILED DESCRIPTION

### Certain Definitions

As used herein and in the appended claims, the singular forms “a,” “and,” and “the” include plural referents unless the context clearly dictates otherwise. Thus, for example, reference to “an agent” includes a plurality of such agents. When ranges are used herein for physical properties, such as molecular weight, or chemical properties, such as chemical formulae, all combinations and subcombinations of ranges and specific embodiments therein are intended to be included. The term “about” when referring to a number or a numerical range means that the number or numerical range referred to is an approximation within experimental variability (or within statistical experimental error), and thus the number or numerical range may vary between 1% and 15% of the stated number or numerical range. The term “substantially the same” when referring to two numbers or ranges means that the numbers or ranges are within between 1% and 15% of each other. The term “comprising” (and related terms such as “comprise” or “comprises” or “having” or “including”) is not intended to exclude, An embodiment of any composition of matter, composition, method, or process, or the like, described herein, may “consist of” or “consist essentially of” the described features.

As used herein, unless stated otherwise, “%” or “percent” as used herein means percent by weight (e.g., w/w %), percent by volume (e.g., v/v %), molar percentage (e.g., mol/mol %). Preferably, “%” or “percent” as used herein means percent by weight (e.g., w/w %).

The terms “treat,” “treating,” or “treatment” as used herein, include reducing, alleviating, abating, ameliorating, managing, relieving, or lessening the symptoms associated with a disease, disease state, condition, or indication (e.g., provided herein) in either a chronic or acute therapeutic scenario. Also, treatment of a disease or disease state described herein includes the disclosure of use of such compound or composition for the treatment of such disease, disease state, disorder, or indication.

### Trough Lung Function

The lung function of a COPD subject typically follows a cycle during maintenance therapy. Lung function in COPD can be measured by determining the forced expiratory volume in one second (FEV₁) of a subject. In the period following administration of the drug, improved lung function may be achieved, reaching a peak improvement a certain period after the drug was administered. The lung function of the subject then decreases towards the trough lung function: i.e. the lowest point in the lung function cycle during maintenance therapy. The trough lung function generally occurs shortly before the next administration of the maintenance therapy drug. For instance, for a twice daily (morning and evening) maintenance therapy, trough lung function generally occurs immediately before the morning dose of the drug (morning trough lung function and evening dose of the drug (evening trough lung function).

The trough lung function achieved during a maintenance therapy may have significant effects on the symptoms and/or quality of life of a COPD subject. The trough lung function effectively represents the worst lung function at a stage during the maintenance treatment. For a twice daily dosing, the morning trough lung function will often coincide with a period of time for which the patient is asleep. This means that the patient can have poor lung function for part of the night, disrupting his or her sleep (for instance due to limited oxygen being absorbed) and causing tiredness, thereby reducing quality of life. In some embodiments, this is the case in COPD subjects who already have disturbed sleep, for instance due to sleep disorders and co-morbidities that disrupt sleep.

If trough lung function is improved, symptoms of COPD may be more consistently ameliorated along with quality of life. Improvement in trough lung function may be beneficial in those subjects already susceptible to sleep disruption. While there are a number of drugs disclosed for use in treating COPD, they are not all equally effective specifically in improving trough lung function, and in particular morning trough lung function. It would be clinically advantageous to administer specific drugs which are effective in improving trough lung function to subjects for whom it is a specific therapeutic desire to optimize the therapeutic effect of the pharmacological intervention.

In some embodiments ensifentrine is effective in increasing trough lung function in subject with or without COPD. In some instances, ensifentrine is effective in increasing trough lung function in subjects with COPD when used as a maintenance therapy. In some embodiments, ensifentrine may improve morning trough lung function in COPD subjects, which may be beneficial for COPD subjects who are susceptible to sleep disruption, for instance due to the presence of a co-morbidity that affects sleep.

### COPD Exacerbations

In some instances, subjects with COPD also suffer from occasional, temporary worsening of their symptoms (known as a COPD exacerbation). A COPD exacerbation typically involves an increase in breathlessness (dyspnea), the presence of excessive mucus (increased sputum volume), change in mucus color (sputum purulence) and/or an increase in coughing. In some instances, the methods of reducing COPD exacerbations provided herein (e.g., such as by administering ensifentrine) improve dyspnea. In some instances, the methods of reducing COPD exacerbations provided herein (e.g., such as by administering ensifentrine) improve excessive mucus production. In some instances, the methods of reducing COPD exacerbations provided herein (e.g., such as by administering ensifentrine) also decrease frequency of coughing.

In some instances, the methods of reducing COPD exacerbations provided herein (e.g., such as by administering a pharmaceutical composition comprising ensifentrine) decrease the frequency of coughing by at least 10% (e.g., at least 20%, at least 30%, at least 40%, at least 50%, at least 60%, or at least 70%), such as compared to as subject who is not be administered a pharmaceutical composition (e.g., comprising ensifentrine) as provided elsewhere herein. In some instances, the methods of reducing COPD exacerbations provided herein (e.g., such as by administering a pharmaceutical composition comprising ensifentrine) decrease the frequency of coughing by at most 95% (e.g., at most 90%, at most 80%, at most 70%, at most 60%, or at most 50%), such as compared to a subject who is not administered a pharmaceutical composition (e.g., comprising ensifentrine) as provided elsewhere herein. In some embodiments, administration of a pharmaceutical composition described herein (e.g., such as comprising ensifentrine) to a subject reduces COPD exacerbations by at least 20%, at least 30%, at least 40%, at least 50%, at least 60%, or at least 70%.

In some instances, certain patient groups are particularly susceptible to COPD exacerbations. In some instances, this includes subjects suffering from additional respiratory conditions such as asthma and subjects suffering from allergic conditions. Subjects susceptible to COPD exacerbations have typically experienced COPD exacerbations previously. In some embodiments, a patient group particularly susceptible to COPD exacerbations includes subjects with asthma. In some embodiments, a patient group particularly susceptible to COPD exacerbations includes subjects suffering from allergic conditions.

In some instances, COPD exacerbations can be severe and limit the quality of life of subjects with COPD. Not all drugs which are disclosed for use in treating COPD are equally effective in reducing the severity and/or frequency of COPD exacerbations or time to first COPD exacerbation. It may be advantageous for a COPD drug to be used which is effective in reducing the severity and/or frequency of COPD exacerbations when there is a specific clinical need to address such exacerbations.

In some embodiments, ensifentrine is highly effective in reducing the frequency and/or severity of COPD exacerbations in subjects with COPD. In some embodiments, ensifentrine has been found to lead to a more pronounced reduction in COPD exacerbations compared to many known treatments for COPD.

### COPD Severity

The Global Initiative for Chronic Obstructive Lung Disease (GOLD) classifies COPD into four distinct stages: mild COPD, moderate COPD, severe COPD, or very severe COPD. The 2022 COPD report is published by the Global Initiative for Chronic Obstructive Disease, Inc, and that document is incorporated by reference herein in its entirety.

In some embodiments, the abovementioned stages of COPD can be classified as set out below, where FVC is forced vital capacity. In some embodiments, mild COPD is classified as FEV₁/FVC<0.7 and FEV₁≥80% predicted. In some embodiments, moderate COPD is classified as FEV₁/FVC<0.7 and 50%≤FEV₁<80% predicted. In some embodiments, severe COPD is classified as FEV₁/FVC<0.7 and 30%≤FEV₁<50% predicted. In some embodiments, very severe COPD is classified as FEV₁/FVC<0.7 and FEV₁<30% predicted.

In some embodiments, the actual FEV₁ for a subject is compared with a predicted FEV₁ value based on factors such as age and height of the subject. These predicted values are readily available to the skilled person, for instance from the National Health and Nutrition Examination Survey III (Hankinson J L, Odencrantz J R, Fedan K B. Spirometry reference values from a sample of the general U.S. Population. Am J Respir Crit Care. 1999; 159:179-187). In some instances, examples of equations for calculating the predicted FEV₁ (in L) for a subject are as follows, where H is height (cm) and A is age (yrs):
Males: 0.0430H-0.0290A-2.490
Females: 0.0395H-0.025A-2.600

The FEV₁ and FVC may be used to determine the severity of COPD in a patient and are measured by carrying out spirometry shortly after administration of an adequate dose of at least one short-acting inhaled bronchodilator. Typically, measurement of FEV₁ and FVC for determining COPD disease severity is done between 15 and 30 minutes following administration of salbutamol (albuterol).

In some embodiments, as used herein, FEV₁ and FVC are determined as set out in the article Standardisation of spirometry, EurJ 2005; 26; 319-338.

In some embodiments, a subject may accordingly be determined to have moderate COPD by measuring FEV₁/FVC<0.7 and 50%≤FEV1<80% predicted FEV₁ value, where FEV₁ is forced expiratory volume in 1 second and FVC is forced vital capacity as measured between 15 and 30 minutes after a dose of a bronchodilator, optionally wherein the bronchodilator is salbutamol. In some embodiments, a subject may accordingly be determined to have severe COPD by measuring FEV₁/FVC<0.7 and 30% FEV₁<50% predicted FEV₁ value, where FEV₁ is forced expiratory volume in 1 second and FVC is forced vital capacity as measured between 15 and 30 minutes after a dose of a bronchodilator, optionally wherein the bronchodilator is salbutamol. The determination of the subject's COPD severity may take place at least 1 day prior to the first administration of the compound (e.g., ensifentrine). The determination of the subject's COPD severity may take place at least 1 week prior to the first administration of the ensifentrine (e.g., or a pharmaceutical composition comprising ensifentrine).

In some instances, as used herein FEV₁ and FVC are determined as set out in the article Standardization of Spirometry, Eur J 2005; 26; 319-338.

In some instances, ensifentrine is effective in treating all forms of COPD. In some instances, ensifentrine is effective in treating moderate COPD. In some instances, ensifentrine is effective in treating mild COPD. In some instances, ensifentrine is effective in treating severe COPD. In some instances, ensifentrine is effective in treating very severe COPD. In some instances, inhaled administration of ensifentrine has been found to result in a significant increase in FEV₁ (forced expiratory volume in 1 second) in subjects with moderate COPD).

A subject provided herein may also be referred to herein as a “patient”. In any of the embodiments provided herein, a subject may be a human subject. In some embodiments, a subject is a male. In some embodiments, a subject is a female. In some embodiments, a subject has an age of greater than or equal to 65 years. In some embodiments, a subject has an age of less than 65 years. In some embodiments, the subjects provided herein may be classified into one or more subgroup populations. In some embodiments, a subject may or may not suffer from chronic bronchitis. In some embodiments, a subject may suffer from moderate COPD. In some embodiments, a subject may have a reversibility status of reversible or non-reversible. In some embodiments, the subject has a blood eosinophil count of less than 150 cells/μL.

In some embodiments, the subjects provided herein may take a background medication. In some embodiments, the background medication is selected from one or more of a long-acting muscarinic antagonist (LAMA), a long-acting beta-agonist (LABA) and an inhaled corticosteroid (ICS). In some embodiments, the background medication is a long-acting muscarinic antagonist (LAMA). In some embodiments, the background medication is a long-acting beta agonist (LABA). In some embodiments, the background medication is an inhaled corticosteroid (ICS). In some embodiments, the subjects provided herein do not take a background medication, which background medication is a long-acting muscarinic antagonist (LAMA), a long-acting beta-agonist (LABA) and an inhaled corticosteroid (ICS).

### Ensifentrine Pharmaceutical Compositions

In some embodiments, provided herein are pharmaceutical compositions comprising the ensifentrine or a pharmaceutically acceptable salt thereof. In some embodiments, provided herein are pharmaceutical compositions comprising ensifentrine free base.

In any of the embodiments provided herein, the pharmaceutical compositions may be liquid pharmaceutical compositions.

In some embodiments, the pharmaceutical composition is a suspension formulation (e.g., a suspension of particles comprising the ensifentrine in a diluent). In some embodiments, the pharmaceutical composition is a dry powder. In some embodiments, the dry powder comprises particles comprising the compound and particles of a carrier (e.g., lactose). In some embodiments, the dry powder is dispersed in a diluent, such as a diluent described elsewhere herein.

In some embodiments, the pharmaceutical compositions provided herein are inhalable pharmaceutical compositions. In some embodiments, the inhalable pharmaceutical composition comprises a suspension of particles of ensifentrine in a diluent, such as a diluent described elsewhere herein.

In some embodiments, the pharmaceutical compositions provided herein comprise (e.g., ensifentrine) particles. In some embodiments, the particles comprise ensifentrine or a pharmaceutically acceptable salt. In some embodiments, the particles comprise at least 90% by weight of ensifentrine or a pharmaceutically acceptable salt thereof. In some embodiments, the particles comprise at least 95% by weight of ensifentrine or a pharmaceutically acceptable salt thereof. In some embodiments, the particles comprise at least 97.5% by weight of ensifentrine or a pharmaceutically acceptable salt thereof. In some embodiments, the particles comprise at least 98% by weight of ensifentrine or a pharmaceutically acceptable salt thereof. In some embodiments, the particles comprise at least 99% by weight of ensifentrine or a pharmaceutically acceptable salt thereof. In some embodiments, the particles comprise at least 99.2% by weight of ensifentrine or a pharmaceutically acceptable salt thereof. In some embodiments, the particles consist of ensifentrine or a pharmaceutically acceptable salt thereof. In some embodiments, the particles comprise at least 90% by weight of ensifentrine free base. In some embodiments, the particles comprise at least 95% by weight of ensifentrine free base. In some embodiments, the particles comprise at least 97.5% by weight of ensifentrine free base. In some embodiments, the particles comprise at least 98% by weight of ensifentrine free base. In some embodiments, the particles comprise at least 99% by weight of ensifentrine free base. In some embodiments, the particles comprise at least 99.2% by weight of ensifentrine free base. In some embodiments, the particles consist of ensifentrine free base.

In some embodiments, a particle comprising ensifentrine as used herein may be referred to as an “ensifentrine particle”.

In some embodiments, the ensifentrine provided herein is in crystalline form. In some embodiments, at least 90% of the ensifentrine by weight is in the form of ensifentrine free base Form I. In some embodiments, at least 90% of the particles comprise ensifentrine free base Form I. Ensifentrine free base Form I is a crystalline polymorph of ensifentrine (crystalline polymorph Form I) which typically has a powder X-ray diffraction pattern comprising characteristic peaks at 10.1° and 12.9°±0.1° 2θ. As used herein, values of °2θ may be measured using an X-ray wavelength of Cu Kα radiation (λ=1.5406 Å). In some embodiments, the powder x-ray diffraction pattern of Form I further comprises characteristic peaks at 15.3° and 17.6°±0.1° 2θ. In some embodiments, Form I of ensifentrine may have a powder X-ray diffraction pattern comprising at least 5 characteristic peaks selected from 6.4°, 10.1°, 12.6°, 12.9°, 13.6°, 14.2°, 14.7°, 15.3°, 15.4°, 15.8°, 17.0°, 17.6°, 18.9°, 20.9°, 22.4°, 22.8° and 28.7°±0.1° 2θ. In some embodiments, crystalline polymorph Form I has a differential scanning calorimetry trace showing a maximum at 248° C.

In some embodiments, the powder x-ray diffraction pattern of Form I of ensifentrine comprises at least 5 (e.g., at least 3, 4, 6, 7, or 8) characteristic peaks, in terms of 2θ, selected from about 6.4°, about 10.1°, about 12.6°, about 12.9°, about 13.6°, about 14.2°, about 14.7°, about 15.3°, about 15.4°, about 15.8°, about 17.0°, about 17.6°, about 18.9°, about 20.9°, about 22.4°, about 22.8°, and about 28.7°.

In some embodiments, the particles comprise at least 98% by weight of ensifentrine crystalline polymorph Form I. In some embodiments, the (e.g., ensifentrine) particles comprise at least 99% by weight of ensifentrine crystalline polymorph Form I.

In some embodiments, ensifentrine crystalline polymorph Form I has the following structural parameters obtained by single crystal analysis:

| Wavelength | 0.71073 Å | |
|---|---|---|
| Crystal System | Triclinic | |
| Space Group | P-1 | |
| Unit Cell Dimensions | a = 8.1246(4) Å | α = 91.583(4)° |
| b | b = 11.4573(5) Å | β = 90.299(4)° |
| c | c = 13.2398(6) Å | γ = 99.628(4)° |
| Volume | 1214.56(10) Å 3 | |
| C | 2. | |

In some instances, the crystalline polymorph Form I is the most thermodynamically stable polymorphic form of ensifentrine and is also expected to have the longest shelf life storage. It is expected that this advantage should extend further to the potential shelf life of any commercial ensifentrine drug product comprising the stable crystalline form I as an API. Furthermore, crystalline polymorph I is more amenable for the development of a reproducible uniform micronized dry solid powder of ensifentrine for certain pharmaceutical formulation applications, such as preparation of formulations described herein.

### Impurity Profile

It has been found that known processes for producing ensifentrine are associated with undesirable levels of impurities. For example, the process described in WO 00/58308 A1 in which a urea group is added using sodium cyanate and aqueous hydrochloric acid produces and ensifentrine substance containing a biuret impurity which could not be readily removed. WO 2018/020249 A1 discusses several possible reagents which may be used to add a urea group in the final step of the production of ensifentrine, but no detailed discussion of the conditions of the final ureation step is provided. It may be desirable to utilize synthetic processes for the production of ensifentrine which produce a drug substance with a favorable impurity profile without the need for extensive purification and recrystallization, and where the drug substance comprises low levels of a biuret impurity. In some instances, it has been found that it is possible to produce ensifentrine with a favorable impurity profile, and ensifentrine having low levels of a biuret impurity, by reaction comprising reacting an amine intermediate with 4-nitrophenyl chloroformate and ammonia in a solvent comprising dichloromethane.

In some embodiments, the (e.g., ensifentrine) particles provided herein comprise 1,3-bis(2-(2-(mesitylimino)-9,10-dimethoxy-4-oxo-6,7-dihydro-2H-pyrimido[6,1-a]isoquinolin-3(4H)-yl)ethyl)urea (BMIQU). In some embodiments, the structure of BMIQU is:

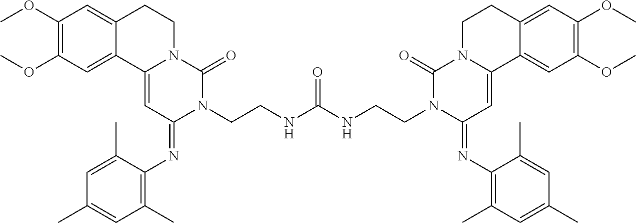

In some embodiments, the (e.g., ensifentrine) particles provided herein comprise the BMIQU in an amount of from 0% to about 0.6% by weight. In some embodiments, the particles comprise BMIQU in an amount of at least about 0.005% by weight. In some embodiments, the particles comprise BMIQU in an amount of at least about 0.01% by weight. In some embodiments, the particles comprise BMIQU in an amount of at least about 0.1% by weight. In some embodiments, the particles comprise BMIQU in an amount of at most about 1% by weight. In some embodiments, the particles comprise BMIQU in an amount of at most about 0.6% by weight. In some embodiments, the particles comprise BMIQU in an amount of at most about 0.3% by weight. In some embodiments, the particles comprise BMIQU in an amount of from about 0.01% to about 0.3% by weight. In some embodiments, the particles comprise BMIQU in an amount of from about 0.3% to about 0.6% by weight. In some embodiments, the particles comprise BMIQU in an amount of from about 0.02% to about 0.06% by weight. In some embodiments, the weight percentage is relative to the total weight of ensifentrine (e.g., free base). In some embodiments, the weight percentage is relative to the total weight of the particles.

In some embodiments, the (e.g., ensifentrine) particles provided herein comprise a biuret impurity of formula (A):

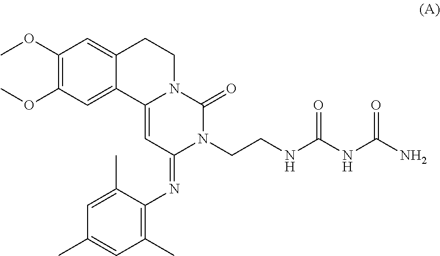 [SMILES: COc1cc2c(cc1OC)-c1c/c(=N\c3c(C)cc(C)cc3C)n(CCNC(=O)NC(N)=O)c(=O)n1CC2]

In some embodiments, the (e.g., ensifentrine) particles comprise the biuret impurity of formula (A) in an amount of from 0% to about 0.5% by weight. In some embodiments, the particles comprise the biuret impurity of formula (A) in an amount of at least 0.01% by weight. In some embodiments, the particles comprise the biuret impurity of formula (A) in an amount of at least 0.05% by weight. In some embodiments, the particles comprise the biuret impurity of formula (A) in an amount of at least 0.1% by weight. In some embodiments, the particles comprise the biuret impurity of formula (A) in an amount of at least 0.3% by weight. In some embodiments, the particles comprise the biuret impurity of formula (A) in an amount of at most 0.7% by weight. In some embodiments, the particles comprise the biuret impurity of formula (A) in an amount of at most 0.5% by weight. In some embodiments, the particles comprise the biuret impurity of formula (A) in an amount of at most 0.3% by weight. In some embodiments, the particles comprise the biuret impurity of formula (A) in an amount of at most 0.2% by weight. In some embodiments, the particles comprise the biuret impurity of formula (A) in an amount of from 0% to about 0.3% by weight. In some embodiments, the particles comprise the biuret impurity of formula (A) in an amount of from 0% to about 0.05% by weight. In some embodiments, the particles comprise the biuret impurity of formula (A) in an amount of from 0% to about 0.03% by weight. In some embodiments, the weight percentage is relative to the total weight of ensifentrine (e.g., free base). In some embodiments, the weight percentage is relative to the total weight of the particles.

In some embodiments, the (e.g., ensifentrine) particles comprise 1-(2-(9-hydroxy-2-(mesitylimino)-10-methoxy-4-oxo-6,7-dihydro-2H-pyrimido[6,1-a]isoquinolin-3(4H)-yl)ethyl)urea (9-des-methyl impurity). In some embodiments, the (e.g., ensifentrine) particles comprise 1-(2-(10-hydroxy-2-(mesitylimino)-9-methoxy-4-oxo-6,7-dihydro-2H-pyrimido[6,1-a]isoquinolin-3(4H)-yl)ethyl)urea (10-des-methyl impurity). In some embodiments, the particles comprise the 9-des-methyl impurity and the 10-des-methyl impurity. In some embodiments, the particles comprise the 9-des-methyl or the 10-des-methyl impurity.

In some embodiments, the 9-des-methyl impurity has the structure:

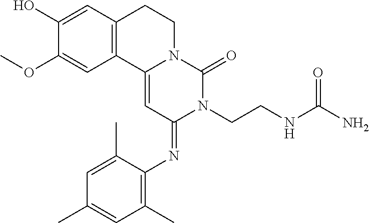 [SMILES: COc1cc2c(cc1O)CCn1c-2c/c(=N\c2c(C)cc(C)cc2C)n(CCNC(N)=O)c1=O]

In some embodiments, the 10-des-methyl impurity has the structure:

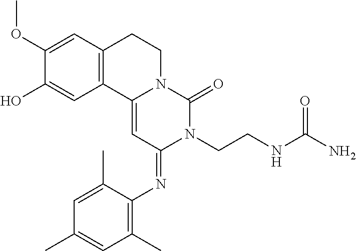 [SMILES: COc1cc2c(cc1O)-c1c/c(=N\c3c(C)cc(C)cc3C)n(CCNC(N)=O)c(=O)n1CC2]

In some embodiments, the (e.g., ensifentrine) particles comprise the 9-des-methyl impurity in an amount of from 0% to about 0.1% by weight. In some embodiments, the particles comprise the 9-des-methyl impurity in an amount of at least 0.01% by weight. In some embodiments, the particles comprise the 9-des-methyl impurity in an amount of at least 0.05% by weight. In some embodiments, the particles comprise the 9-des-methyl impurity in an amount of at least 0.1% by weight. In some embodiments, the particles comprise the 9-des-methyl impurity in an amount of at most 0.2% by weight. In some embodiments, the particles comprise the 9-des-methyl impurity in an amount of 0.1% by weight. In some embodiments, the particles comprise the 9-des-methyl impurity in an amount of from about 0.01% to about 0.1% by weight. In some embodiments, the particles comprise the 9-des-methyl impurity in an amount of from about 0.01% to about 0.05% by weight. In some embodiments, the weight percentage is relative to the total weight of ensifentrine (e.g., free base). In some embodiments, the weight percentage is relative to the total weight of the particles.

In some embodiments, the (e.g., ensifentrine) particles comprise the 10-des-methyl impurity in an amount of from 0% to about 0.1% by weight. In some embodiments, the particles comprise the 10-des-methyl impurity in an amount of at least 0.01% by weight. In some embodiments, the particles comprise the 10-des-methyl impurity in an amount of at least 0.05% by weight. In some embodiments, the particles comprise the 10-des-methyl impurity in an amount of at least 0.1% by weight. In some embodiments, the particles comprise the 10-des-methyl impurity in an amount of at most 0.2% by weight. In some embodiments, the particles comprise the 10-des-methyl impurity in an amount of 0.1% by weight. In some embodiments, the particles comprise the 10-des-methyl impurity in an amount of from about 0.01% to about 0.1% by weight. In some embodiments, the particles comprise the 10-des-methyl impurity in an amount of from about 0.01% to about 0.05% by weight. In some embodiments, the weight percentage is relative to the total weight of ensifentrine (e.g., free base). In some embodiments, the weight percentage is relative to the total weight of the particles.

In some embodiments, the (e.g., ensifentrine) particles comprise (E)-2-(mesitylimino)-9,10-dimethoxy-2,3,6,7-tetrahydro-4H-pyrimido[6,1-a]isoquinolin-4-one (compound (I)). In some embodiments, the (e.g., ensifentrine) particles comprise (E)-3-(2-aminoethyl)-2-(mesitylimino)-9,10-dimethoxy-2,3,6,7-tetrahydro-4H-pyrimido[6,1-a]isoquinolin-4-one (compound (IV)). In some embodiments, the particles comprise both compound (I) and compound (IV). In some embodiments, the particles comprise compound (I) or compound (IV).

In some embodiments, the structure of compound (I) is:

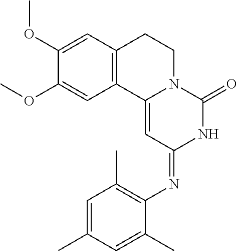 [SMILES: COc1cc2c(cc1OC)-c1c/c(=N\c3c(C)cc(C)cc3C)[nH]c(=O)n1CC2]

In some embodiments, the structure of compound (IV) is:

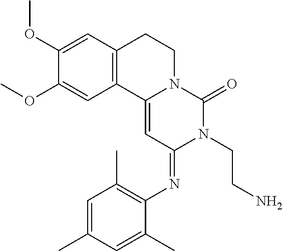 [SMILES: COc1cc2c(cc1OC)-c1c/c(=N\c3c(C)cc(C)cc3C)n(CCN)c(=O)n1CC2]

In some embodiments, the (e.g., ensifentrine) particles comprise from 0% to about 0.04% by weight of compound (I). In some embodiments, the particles comprise at least 0.01% by weight of compound (I). In some embodiments, the particles comprise at least 0.02% by weight of compound (I). In some embodiments, the particles comprise at most 0.05% by weight of compound (I). In some embodiments, the particles comprise at most 0.04% by weight of compound (I). In some embodiments, the (e.g., ensifentrine) particles comprise from 0% to about 0.04% by weight of compound (IV). In some embodiments, the particles comprise at least 0.01% by weight of compound (IV). In some embodiments, the particles comprise at least 0.02% by weight of compound (IV). In some embodiments, the particles comprise at most 0.05% by weight of compound (IV). In some embodiments, the particles comprise at most 0.04% by weight of compound (IV). In some embodiments, the particles do not comprise compound (I). In some embodiments, the particles do not comprise compound (IV). In some embodiments, the particles do not comprise compound (I) or compound (IV). In some embodiments, the (e.g., ensifentrine) particles comprise from 0% to about 0.04% by weight total of compound (I) and compound (IV). In some embodiments, the particles comprise at least 0.01% by weight total of compound (I) and compound (IV). In some embodiments, the particles comprise at least 0.02% by weight total of compound (I) and compound (IV). In some embodiments, the particles comprise at most 0.05% by weight total of compound (I) and compound (IV). In some embodiments, the particles comprise at most 0.04% by weight total of compound (I) and compound (IV). In some embodiments, the weight percentage is relative to the total weight of ensifentrine (e.g., free base). In some embodiments, the weight percentage is relative to the total weight of the particles.

In some embodiments, the BMIQU, biuret impurity, 9-des-methyl impurity, 10-des-methyl impurity, compound (I), and compound (IV), if present, may optionally be present in the form of a salt. In certain embodiments, the BMIQU, biuret impurity, 9-des-methyl impurity, 10-des-methyl impurity, compound (I), and compound (IV) are present in free base form.

In some embodiments, the (e.g., ensifentrine) particles provided herein comprise ensifentrine (e.g., ensifentrine free base (e.g., in crystalline Form 1)), BMIQU, biuret impurity, 9-des-methyl impurity, and 10-des-methyl impurity. In some embodiments, the (e.g., ensifentrine) particles provided herein consists of ensifentrine (e.g., ensifentrine free base (e.g., in crystalline Form 1)), BMIQU, biuret impurity, 9-des-methyl impurity, and 10-des-methyl impurity.

In some embodiments, the (e.g., ensifentrine) particles provided herein comprise from about 99.4% to about 99.9% by weight of ensifentrine, from about 0.01% to about 0.3% by weight of BMIQU, from 0% to about 0.1% by weight of the biuret impurity, from about 0.01% to about 0.2% by weight of the 9-des-methyl impurity, and from about 0.01% to about 0.2% by weight of the 10-des-methyl impurity, wherein the weight percent is relative to the total weight of the particles.

In some embodiments, the (e.g., ensifentrine) particles provided herein comprise from about 99.5% to about 99.9% by weight of ensifentrine, from about 0.02% to about 0.1% by weight of BMIQU, from 0% to about 0.04% by weight of the biuret impurity, from about 0.01% to about 0.1% by weight of the 9-des-methyl impurity, and from about 0.01% to about 0.1% by weight of the 10-des-methyl impurity, wherein the weight percent is relative to the total weight of the particles.

In some embodiments, the (e.g., ensifentrine) particles provided herein consists of from about 99.6% to about 99.9% by weight of ensifentrine, from about 0.02% to about 0.1% by weight of BMIQU, from 0% to about 0.04% by weight of the biuret impurity, from about 0.01% to about 0.1% by weight of the 9-des-methyl impurity, and from about 0.01% to about 0.1% by weight of the 10-des-methyl impurity, wherein the weight percent is relative to the total weight of the particles.

In some embodiments, provided herein are particles comprising ensifentrine (e.g., ensifentrine free base). In some embodiments, the particles have a particle size distribution with a Dv50 of from about 0.5 μm to about 5.0 μm. In some embodiments, the particles comprise a Dv50 of from about 1.0 μm to about 2.0 μm. In some embodiments, the particles comprise a Dv90 of from about 1.8 μm to about 2 μm. In some embodiments, the particles comprise a Dv90 of from about 2.5 μm to about 6.0 μm. In some embodiments, the particles comprise a Dv90 of from about 2.8 μm to about 4.2 μm. In some embodiments, the particles comprise a Dv10 of from about 0.2 μm to about 1.0 μm In some embodiments, the particles comprise a Dv10 of from about 0.4 μm to about 0.6 μm. In some embodiments, the particles comprise a Dv10 of from about 0.4 μm to about 0.6 μm and a Dv90 of from about 2.8 μm to about 4.2 μm. In certain embodiments, the particles comprise a Dv90 that is not more than 4.5 μm. In some embodiments, the particles comprise a Dv90 of about 2, 2.1, 2.2, 2.3, 2.5, 2.5, or 2.6 μm.

In some embodiments, particle sizes are described herein by reference to the Dv50 value, which is the median particle size for a volume distribution. Thus, half of the volume of the particles have diameters of less than the Dv50 value and half the volume of the particles have diameters of greater than the Dv50 value. Similarly, in some embodiments, particle sizes are described herein by their Dv90 or Dv10 values. The skilled artisan would understand this to be a well-known parameter in which to describe particle size distributions.

The technique used to measure particle size (e.g., Dv10, Dv50, Dv90 values) as described herein may be laser diffraction.

The technique used to measure the Dv50 values as stated herein is typically laser diffraction. For instance, the particle size distribution can be measured by laser diffraction using a Malvern Mastersizer 3000. Typically, the instrument parameters for the Malvern Mastersizer 3000 are as follows:
Non-spherical particle mode: Yes
Refractive index: 1.500
Absorption index: 0.500
Particle density: 1.00 g/cm³
Dispersant name: 0.02% Polysorbate 20 in water
Refractive index: 1.330
Stirrer; 1000 rpm
Sample measurement: 10 s
Obscuration: 10-20%

The particles described herein may comprise ensifentrine (e.g., ensifentrine free base). In some embodiments, the particles comprise at least 90 wt % ensifentrine free base relative to the total weight of the particles. In some embodiments, the particles comprise at least 95 wt % ensifentrine. In some embodiments, the particles comprise at least 97 wt % ensifentrine. In some embodiments, the particles comprise at least 99 wt % ensifentrine. In some embodiments, the particles comprise at least 99.5 wt % ensifentrine. In some instances, the particles consist of ensifentrine.

The (e.g., ensifentrine) particles comprising ensifentrine (e.g., ensifentrine free base) may be present in the pharmaceutical compositions in any suitable concentration according to one of skill in the art. In some embodiments, particles comprising the ensifentrine are present in the pharmaceutical compositions at a concentration of about 0.1 mg/mL to about 5.0 mg/mL. In some embodiments, the particles are present in the pharmaceutical compositions at a concentration of about 0.1 mg/mL to about 2.5 mg/mL. In some embodiments, the particles are present in the pharmaceutical compositions at a concentration of about 1.0 mg/mL to about 2.0 mg/mL. In some embodiments, the particles comprising ensifentrine are present in the pharmaceutical compositions at a concentration of (e.g., about) 1.2 mg/mL.

In some embodiments, the concentration of ensifentrine or a pharmaceutically acceptable salt thereof (e.g., ensifentrine free base) in the pharmaceutical composition is at least 0.8 mg/mL. In some embodiments, the concentration of ensifentrine or a pharmaceutically acceptable salt thereof (e.g., ensifentrine free base) is at least about 0.9 mg/mL (e.g., 1 mg/mL, 1.1 mg/mL). In some embodiments, the concentration of ensifentrine or a pharmaceutically acceptable salt thereof (e.g., ensifentrine free base) is at most 1.5 mg/mL. the concentration of ensifentrine or a pharmaceutically acceptable salt thereof (e.g., ensifentrine free base) is at most 1.4 mg/mL (e.g., 1.3 mg/mL). In some embodiments, the concentration of ensifentrine or a pharmaceutically acceptable salt thereof (e.g., ensifentrine free base) is from about 1 mg/mL to about 1.4 mg/mL (e.g., 1.1 mg/mL to 1.3 mg/mL). In certain embodiments, the concentration of ensifentrine or a pharmaceutically acceptable salt thereof (e.g., ensifentrine free base) is about 1.2 mg/mL. In some embodiments, the concentration of ensifentrine free base in the pharmaceutical composition is (e.g., about) 1.2 mg/mL.

In some embodiments, the pharmaceutical compositions provided herein further comprise one or more tonicity adjusters, one or more buffers, and one or more surfactants. In some embodiments, the pharmaceutical composition further comprises one or more tonicity adjusters. In some embodiments, the pharmaceutical composition further comprises one or more buffers. In some embodiments, the pharmaceutical composition further comprises one or more surfactants.

In some embodiments, the pharmaceutical compositions provided herein further comprise one or more tonicity adjusters. In some embodiments, the tonicity adjuster comprises sodium chloride. In some embodiments, the tonicity adjuster (e.g., sodium chloride) is present in the pharmaceutical composition at a concentration of at least 1 mg/mL (e.g., 2 mg/mL, 3 mg/mL, 4 mg/mL, 5 mg/mL, 6 mg/mL, 7 mg/mL). In some embodiments, the tonicity adjuster (e.g., sodium chloride) is present in the pharmaceutical composition at a concentration of at most 15 mg/mL (e.g., 14 mg/mL, 13 mg/mL, 12 mg/mL, 11 mg/mL, 10 mg/mL, 9 mg/mL). In some embodiments, the tonicity adjuster (e.g., sodium chloride) is present in the pharmaceutical composition at a concentration of from about 1 mg/mL to about 15 mg/mL, about 3 mg/mL to about 10 mg/mL, about 4 mg/mL to about 10 mg/mL, about 5 mg/mL to about 11 mg/mL, about 5 mg/mL to about 9 mg/mL, or about 6 mg/mL to about 9 mg/mL. In some embodiments, the tonicity adjuster (e.g., sodium chloride) is present in the pharmaceutical composition at a concentration of about 7 mg/mL to about 10 mg/mL. In some embodiments, the tonicity adjuster (e.g., sodium chloride) is present in the pharmaceutical composition at a concentration of 8 mg/mL to 9 mg/mL. In certain embodiments, the tonicity adjuster (e.g., sodium chloride) is present in the pharmaceutical composition at a concentration of (e.g., about) 8.6 mg/mL.

In some embodiments, the pharmaceutical compositions provided herein further comprise one or more buffers. In some embodiments, the one or more buffers comprise one or more of a citrate buffer, a phosphate buffer, an acetate buffer, and/or a bicarbonate buffer. In certain embodiments, the buffer is a phosphate buffer. In some embodiments, the buffer comprises sodium dihydrogen phosphate dihydrate and/or disodium hydrogen phosphate dihydrate. In some embodiments, the buffer (e.g., sodium dihydrogen phosphate dihydrate and/or disodium hydrogen phosphate dihydrate) is present in the pharmaceutical composition at a concentration of at least 0.1 mg/mL (e.g., 0.2 mg/mL, 0.4 mg/mL, 0.8 mg/mL, 1 mg/mL, or 1.4 mg/mL). In some embodiments, the buffer (e.g., sodium dihydrogen phosphate dihydrate and/or disodium hydrogen phosphate dihydrate) is present in the pharmaceutical composition at a concentration of 4 mg/mL (e.g., 3.8 mg/mL, 3.3 mg/mL, 2.8 mg/mL, 2.5 mg/mL, or 2 mg/mL). In some embodiments, the buffer (e.g., sodium dihydrogen phosphate dihydrate and/or disodium hydrogen phosphate dihydrate) is present in the pharmaceutical composition at a concentration of about 1 mg/mL to about 2 mg/mL, about 1.2 mg/mL to about 1.8 mg/mL, or about 1.4 mg/mL to about 1.7 mg/mL. In some embodiments, the buffer (e.g., sodium dihydrogen phosphate dihydrate and/or disodium hydrogen phosphate dihydrate) is present in the pharmaceutical composition at a concentration of about 1.5 mg/mL to about 1.7 mg/mL.

In some embodiments, the pharmaceutical composition comprises sodium dihydrogen phosphate dihydrate at a concentration of at least 0.1 mg/mL (e.g., 0.2 mg/mL, 0.3 mg/mL, 0.4 mg/mL, 0.5 mg/mL, 0.6 mg/mL). In some embodiments, the pharmaceutical composition comprises sodium dihydrogen phosphate dihydrate at a concentration of at most 1.2 mg/mL (e.g., 1.1 mg/mL, 1.0 mg/mL, 0.9 mg/mL, 0.8 mg/mL). In some embodiments, the pharmaceutical composition comprises sodium dihydrogen phosphate dihydrate at a concentration of from about 0.5 mg/mL to about 0.9 mg/mL. In some embodiments, the pharmaceutical composition comprises sodium dihydrogen phosphate dihydrate at a concentration of about 0.7 mg/mL to about 0.8 mg/mL. In some embodiments, the pharmaceutical composition comprises sodium dihydrogen phosphate dihydrate at a concentration of about 0.7 mg/mL. In certain embodiments, the pharmaceutical composition comprises sodium dihydrogen phosphate dihydrate at a concentration of (e.g., about) 0.744 mg/mL.

In some embodiments, the pharmaceutical composition comprises disodium hydrogen phosphate dihydrate at a concentration of at least 0.1 mg/mL (e.g., 0.2 mg/mL, 0.3 mg/mL, 0.4 mg/mL, 0.5 mg/mL, 0.6 mg/mL, 0.7 mg/mL). In some embodiments, the pharmaceutical composition comprises disodium hydrogen phosphate dihydrate at a concentration of at most 1.2 mg/mL (e.g., 1.1 mg/mL, 1.0 mg/mL, 0.9 mg/mL). In some embodiments, the pharmaceutical composition comprises disodium hydrogen phosphate dihydrate at a concentration of from about 0.7 mg/mL to about 1 mg/mL. In some embodiments, the pharmaceutical composition comprises disodium hydrogen phosphate dihydrate at a concentration of about 0.8 mg/mL to about 0.9 mg/mL. In some embodiments, the pharmaceutical composition comprises disodium hydrogen phosphate dihydrate at a concentration of about 0.8 mg/mL. In some embodiments, the pharmaceutical composition comprises disodium hydrogen phosphate dihydrate at a concentration of about 0.9 mg/mL. In certain embodiments, the pharmaceutical composition comprises disodium hydrogen phosphate dihydrate at a concentration of (e.g., about) 0.853 mg/mL.

In some embodiments, the pharmaceutical compositions provided herein further comprise one or more surfactants. In some embodiments, the one or more surfactants comprise one or more of lecithin, oleic acid, polyoxyethylene glycol alkyl ethers (e.g., PEG 300, PEG 600, PEG 1000, Brij 30, Brij 35, Brij 56, Brij 76 and Brij 97), polypropylene glycol (e.g., PPG 2000), glucoside alkyl ethers, polyoxyethylene glycol octylphenol ethers, polyoxyethylene glycol alkylphenol ethers, glycerol alkyl esters, polyoxyethylene glycol sorbitan alkyl esters (e.g., polysorbates, for instance polysorbate 20, polysorbate 40, polysorbate 60 and polysorbate 80), sorbitan alkyl esters (e.g., sorbitan monolaurate (Span 20), sorbitan monooleate (Span 80) and sorbitan trioleate (Span 85)), cocamide MEA, cocamide DEA, dodecyldimethylamine oxide, block copolymers of polyethylene glycol and polypropylene glycol (poloxamers), block copolymers of polyethylene glycol and polypropylene oxide (e.g., Pluronic surfactants), polyvinyl pyrrolidone K25, polyvinyl alcohol, oligolactic acid, sodium dioctyl sulfosuccinate and polyethoxylated tallow amine (POEA). In certain embodiments, the one or more embodiments, comprise a polysorbate and/or a sorbitan alkyl ester. In some embodiments, the one or more surfactants comprise polysorbate 20 (polyoxyethylene (20) sorbitan monolaurate), polysorbate 40 (polyoxyethylene (20) sorbitan monopalmitate), polysorbate 60 (polyoxyethylene (20) sorbitan monostearate) or polysorbate 80 (polyoxyethylene (20) sorbitan monooleate). In some embodiments, the one or more surfactants comprise sorbitan monolaurate (Span 20), sorbitan monooleate (Span 80) or sorbitan trioleate (Span 85). In some embodiments, the pharmaceutical composition comprises polysorbate 20 (Tween 20) and/or sorbitan monolaurate (Span 20). In some instances, the surfactants act as wetting agents in the (e.g., liquid) pharmaceutical compositions described herein.

In some embodiments, the pharmaceutical composition comprises the one or more surfactants (e.g., polysorbate 20 and/or sorbitan monolaurate) in a concentration of at least 0.05 mg/mL (e.g., 0.1 mg/mL, 0.2 mg/mL, 0.3 mg/mL, 0.4 mg/mL). In some embodiments, the pharmaceutical composition comprises the one or more surfactants (e.g., polysorbate 20 and/or sorbitan monolaurate) in a concentration of at most 1 mg/mL (e.g., 0.9 mg/mL, 0.8 mg/mL, 0.7 mg/mL, 0.6 mg/mL). In some embodiments, the pharmaceutical composition comprises the one or more surfactants (e.g., polysorbate 20 and/or sorbitan monolaurate) in a concentration of from about 0.3 mg/mL to about 0.7 mg/mL. In some embodiments, the pharmaceutical composition comprises the one or more surfactants (e.g., polysorbate 20 and/or sorbitan monolaurate) at a concentration of from about 0.01 mg/mL to 2 mg/mL. In some embodiments, the pharmaceutical composition comprises the one or more surfactants (e.g., polysorbate 20 and/or sorbitan monolaurate) in a concentration of from about 0.4 mg/mL to about 0.6 mg/mL. In some embodiments, the pharmaceutical composition comprises the one or more surfactants (e.g., polysorbate 20 and/or sorbitan monolaurate) at a concentration of about 0.5 mg/mL. In some embodiments, the pharmaceutical composition comprises the one or more surfactants (e.g., polysorbate 20 and/or sorbitan monolaurate) at a concentration of about 0.6 mg/mL. In certain embodiments, the pharmaceutical composition comprises the one or more surfactants (e.g., polysorbate 20 and/or sorbitan monolaurate) in a concentration of (e.g., about) 0.55 mg/mL.

In some embodiments, the pharmaceutical composition comprises polysorbate 20 (e.g., Tween 20) in a concentration of at least 0.1 mg/mL (e.g., 0.2 mg/mL, 0.3 mg/mL, 0.4 mg/mL). In some embodiments, the pharmaceutical composition comprises polysorbate 20 (e.g., Tween 20) in a concentration of at most 1 mg/mL (e.g., 0.9 mg/mL, 0.8 mg/mL, 0.7 mg/mL, 0.6 mg/mL). In some embodiments, the pharmaceutical composition comprises polysorbate 20 (e.g., Tween 20) in a concentration of from about 0.3 mg/mL to about 0.7 mg/mL. In some embodiments, the pharmaceutical composition comprises polysorbate 20 (e.g., Tween 20) in a concentration of about 0.4 mg/mL to about 0.6 mg/mL. In certain embodiments, the pharmaceutical composition comprises polysorbate 20 (e.g., Tween 20) in a concentration of about 0.5 mg/mL.

In some embodiments, the pharmaceutical composition comprises sorbitan monolaurate (Span 20) in a concentration of at least 0.01 mg/mL (e.g., 0.02 mg/mL, 0.03 mg/mL, 0.04 mg/mL). In some embodiments, the pharmaceutical composition comprises sorbitan monolaurate (Span 20) in a concentration of at most 0.1 mg/mL (e.g., 0.09 mg/mL, 0.08 mg/mL, 0.07 mg/mL, 0.06 mg/mL). In some embodiments, the pharmaceutical composition comprises sorbitan monolaurate (Span 20) in a concentration of from about 0.03 mg/mL to about 0.07 mg/mL. In some embodiments, the pharmaceutical composition comprises sorbitan monolaurate (Span 20) in a concentration of from about 0.04 mg/mL to about 0.06 mg/mL. In certain embodiments, the pharmaceutical composition comprises sorbitan monolaurate (Span 20) in a concentration of (e.g., about) 0.05 mg/mL.

In some embodiments, the pharmaceutical compositions provided herein comprise:
a. water;
b. particles consisting of ensifentrine free base at a concentration of from about 0.1 mg/mL to about 20 mg/mL;
c. one or more tonicity adjusters at a concentration of from about 1.0 mg/mL to about 15 mg/mL;
d. one or more buffers at a total concentration of from about 0.1 mg/mL to about 4.0 mg/mL; and
e. one or more surfactants at total concentration of from about 0.05 mg/mL to about 3 mg/mL.

In some embodiments, the liquid pharmaceutical compositions provided herein comprise:
a. water;
b. particles consisting of ensifentrine free base at a concentration of from about 0.5 mg/mL to about 6 mg/mL;
c. sodium chloride at a concentration of from about 5 mg/mL to about 12 mg/mL;
d. sodium dihydrogen phosphate dihydrate at a concentration of from about 0.3 mg/mL to about 2 mg/mL;
e. disodium hydrogen phosphate dihydrate at a concentration of from about 0.3 mg/mL to about 2 mg/mL; and
f. sorbitan monolaurate at a concentration of from about 0.01 mg/mL to about 0.5 mg/mL.

In some instances, the pharmaceutical compositions provided herein may be referred to as ensifentrine compositions.

In some embodiments, the pharmaceutical compositions herein are provided in an ampule, such as for administration by inhalation using a nebulizer. In some embodiments, the ampule comprises the suspension of ensifentrine particles as described herein. In some embodiments, the ampule comprises about 2.6 mL of the pharmaceutical composition. In some embodiments, the ampule comprises at least 3 mg of ensifentrine (e.g., ensifentrine free base). In some embodiments, the ampule comprises about 3.1 mg of ensifentrine. In some embodiments, the ampule is a low density polyethylene (LDPE) ampule.

In some embodiments, ensifentrine or a pharmaceutically acceptable salt as described herein may be used in combination with a second active agent. In some embodiments, the ensifentrine or pharmaceutically salt thereof may be administered separately or simultaneously with the second active agent. In some embodiments, the subject may already be taking a second active agent as a background therapy for COPD. Alternatively, treatment with the second active agent may start at around the same time as treatment with ensifentrine. The ensifentrine and the second active agent may be administered in a fixed combination.

In some embodiments, the second active agent as provide herein is a muscarinic receptor antagonist, a beta adrenergic receptor agonist, or an inhaled corticosteroid. In some embodiments, the ensifentrine or pharmaceutically acceptable salt may be used in combination with muscarinic receptor antagonist, a beta adrenergic receptor agonist, or an inhaled corticosteroid. The second active agent may be a long-acting muscarinic receptor antagonist (LAMA) or a long-acting beta-adrenergic receptor agonist (LABA). In some embodiments, the second active agent is a muscarinic receptor antagonist. In some embodiments, the second active agent is a beta adrenergic receptor agonist. In some embodiments, the second active agent is an inhaled corticosteroid.

In some embodiments, non-limiting examples of LAMAs include aclidinium, darotropium, tiotropium, glycopyrrolate and umeclidinium. In some embodiments, non-limiting examples of LABAs include salmeterol, formoterol, indacaterol, vilanterol, olodaterol, abediterol and carmoterol. In some embodiments, non-limiting examples of inhaled corticosteroids include beclomethosone, budesonide, fluticasone propionate, ciclesonide, mometasone and fluticasone furoate.

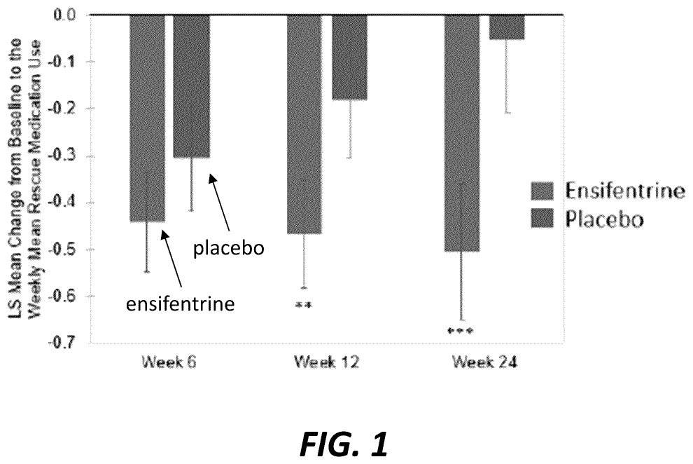

In some embodiments, the subjects provided herein may be using a beta-agonist, such as salbutamol, as a rescue medication. In some embodiments, provided herein are methods of reducing the necessity for a rescue medication. In some embodiments, administration of a pharmaceutical composition as provided herein reduces the necessity for use of a rescue medication. In some embodiments, the administration of a pharmaceutical composition provided herein results in a statistically significant decrease in rescue mediation use (e.g., average daily puffs over a week), such as over 24 weeks. In some instances, the average daily rescue medication use decreases incrementally with the length that the pharmaceutical composition is administered. In some instances, an individual administered with a pharmaceutical composition for 24 weeks has a higher decrease in rescue mediation use as compared to a placebo than an individual administered with a pharmaceutical composition for 6 weeks (e.g., see FIG. 1). In some instances, an individual administered a pharmaceutical composition for 6 weeks has a higher decrease in rescue mediation use as compared to placebo than an individual administered with a pharmaceutical composition for 24 weeks (e.g., see FIG. 1). In certain embodiments, an individual administered with a pharmaceutical composition exhibits reduced rescue medication use (average puffs over a week) in comparison to a subject being administered a placebo.

### Methods of Treating COPD

Provided herein are methods of treating chronic obstructive pulmonary disease (COPD) (e.g., of varying severity), such as COPD provided elsewhere herein. In some embodiments, the methods provided herein comprise administering to a subject in need thereof an (e.g., liquid) pharmaceutical composition, such as a pharmaceutical composition provided elsewhere herein, comprising a therapeutically effective amount of ensifentrine. In some embodiments, the methods provided herein comprise administering to the subject in need thereof (e.g., a therapeutically effective amount of) ensifentrine.

Methods of measuring improvement in COPD may include, but are not limited to one or more of FEV₁, evaluating respiratory symptoms (E-RS), transition dyspnea index (TDI), and quality of life (e.g., using St. George's respiratory questionnaire (SGRQ) or St. George's respiratory questionnaire for COPD patients (SGRQ-C)).

In some embodiments, the methods provided herein are effective in treating mild COPD, moderate COPD, severe COPD, or very severe COPD. In some embodiments, the methods provided herein are effective in treating mild COPD. In some embodiments, the methods provided herein are effective in treating moderate COPD. In some embodiments, the methods provided herein are effective in treating severe COPD. In some embodiments, the methods provided herein are effective in treating very severe COPD. In some embodiments, the methods provided herein are effective in treating moderate or severe COPD, such as moderate or severe COPD such as classified as described elsewhere herein.

As used herein, AUC₀₋ₜₐᵤ is the AUC from administration to the end of the dosing period (which may for instance be 8 h, 12 h or 24 h). As used herein, tau is typically 12 h and AUC₀₋ₜₐᵤ is AUC_{0-12 h}.

In some embodiments, administration of a pharmaceutical composition (e.g., comprising ensifentrine) to a subject having mild or moderate COPD provides a FEV₁ AUC_{0-12 h}(e.g., after 12 weeks of administration) of at least 0.08 L (80 mL). In some embodiments, administration of a pharmaceutical composition (e.g., comprising ensifentrine) to a subject having mild or moderate COPD provides a FEV₁ AUC_{0-12 h} (e.g., after 12 weeks of administration) of at least 0.09 L (e.g., 0.1 L, 0.11 L). In some embodiments, administration of a pharmaceutical composition (e.g., comprising ensifentrine) to a subject having mild or moderate COPD provides a FEV₁ AUC_{0-12 h} (e.g., after 12 weeks of administration) of at most 0.14 L (140 mL). In some embodiments, administration of a pharmaceutical composition (e.g., comprising ensifentrine) to a subject having mild or moderate COPD provides a FEV₁ AUC_{0-12 h} (e.g., after 12 weeks of administration) of at most 0.13 L (e.g., 0.12 L, 0.11 L). In some embodiments, administration of a pharmaceutical composition (e.g., comprising ensifentrine) to a subject having mild or moderate COPD provides a FEV₁ AUC_{0-12 h} (e.g., after 12 weeks of administration) of about 0.08 L to about 0.14 L. In some embodiments, administration of a pharmaceutical composition (e.g., comprising ensifentrine) to a subject having mild or moderate COPD provides a FEV₁ AUC_{0-12 h} (e.g., after 12 weeks of administration) of about 0.09 L to about 0.13 L. In some embodiments, administration of a pharmaceutical composition (e.g., comprising ensifentrine) to a subject having mild or moderate COPD provides a FEV₁ AUC_{0-12 h} (e.g., after 12 weeks of administration) of about 0.11 L (110 mL).

In some embodiments, administration of a pharmaceutical composition (e.g., comprising ensifentrine) to a subject having severe or very severe COPD provides a FEV₁ AUC_{0-12 h} (e.g., after 12 weeks of administration) of at least 0.03 L (30 mL). In some embodiments, administration of a pharmaceutical composition (e.g., comprising ensifentrine) to a subject having severe or very severe COPD provides a FEV₁ AUC_{0-12 h} (e.g., after 12 weeks of administration) of 0.04 L (e.g., 0.05 L, 0.06 L). In some embodiments, administration of a pharmaceutical composition (e.g., comprising ensifentrine) to a subject having severe or very severe COPD provides a FEV₁ AUC_{0-12 h} (e.g., after 12 weeks of administration) of at most 0.09 L (e.g., 0.08 L, 0.07 L, 0.06 L). In some embodiments, administration of a pharmaceutical composition (e.g., comprising ensifentrine) to a subject having severe or very severe COPD provides a FEV₁ AUC_{0-12 h} (e.g., after 12 weeks of administration) of about 0.03 L to about 0.09 L. In some embodiments, administration of a pharmaceutical composition (e.g., comprising ensifentrine) to a subject having severe or very severe COPD provides a FEV₁ AUC_{0-12 h} (e.g., after 12 weeks of administration) of about 0.05 L to about 0.07 L. In some embodiments, administration of a pharmaceutical composition (e.g., comprising ensifentrine) to a subject having severe or very severe COPD provides a FEV₁ AUC_{0-12 h} (e.g., after 12 weeks of administration) of about 0.06 L (60 mL).

In some embodiments, administration of a pharmaceutical composition (e.g., comprising ensifentrine) to a subject having mild or moderate COPD provides a change from baseline FEV₁ to peak FEV₁ (e.g., after 12 weeks of administration) of at least 0.14 L (e.g., 140 mL). In some embodiments, administration of a pharmaceutical composition (e.g., comprising ensifentrine) to a subject having mild or moderate COPD provides a change from baseline FEV₁ to peak FEV₁ (e.g., after 12 weeks of administration) of at least 0.15 L (e.g., 0.16 L, 0.17 L). In some embodiments, administration of a pharmaceutical composition (e.g., comprising ensifentrine) to a subject having mild or moderate COPD provides a change from baseline FEV₁ to peak FEV₁ (e.g., after 12 weeks of administration) of at most 0.2 L (e.g., 0.19 L, 0.18 L, 0.17 L). In some embodiments, administration of a pharmaceutical composition (e.g., comprising ensifentrine) to a subject having mild or moderate COPD provides a change from baseline FEV₁ to peak FEV₁ (e.g., after 12 weeks of administration) of about 0.14 L to about 0.2 L. In some embodiments, administration of a pharmaceutical composition (e.g., comprising ensifentrine) to a subject having mild or moderate COPD provides a change from baseline FEV₁ to peak FEV₁ (e.g., after 12 weeks of administration) of about 0.15 L to about 0.18 L. In some embodiments, administration of a pharmaceutical composition (e.g., comprising ensifentrine) to a subject having mild or moderate COPD provides a change from baseline FEV₁ to peak FEV₁ (e.g., after 12 weeks of administration) of about 0.17 L (170 mL).

In some embodiments, administration of a pharmaceutical composition (e.g., comprising ensifentrine) to a subject having severe or very severe COPD provides a change from baseline FEV₁ to peak FEV₁ (e.g., after 12 weeks of administration) of at least 0.08 L (80 mL). In some embodiments, administration of a pharmaceutical composition (e.g., comprising ensifentrine) to a subject having severe or very severe COPD provides a change from baseline FEV₁ to peak FEV₁ (e.g., after 12 weeks of administration) of at least 0.09 L (e.g., 0.1 L, 0.11 L). In some embodiments, administration of a pharmaceutical composition (e.g., comprising ensifentrine) to a subject having severe or very severe COPD provides a change from baseline FEV₁ to peak FEV₁ (e.g., after 12 weeks of administration) of at most 0.015 L (150 mL). In some embodiments, administration of a pharmaceutical composition (e.g., comprising ensifentrine) to a subject having severe or very severe COPD provides a change from baseline FEV₁ to peak FEV₁ (e.g., after 12 weeks of administration) of at most 0.15 L (e.g., 0.14 L, 0.13 L, or 0.12 L). In some embodiments, administration of a pharmaceutical composition (e.g., comprising ensifentrine) to a subject having severe or very severe COPD provides a change from baseline FEV₁ to peak FEV₁ (e.g., after 12 weeks of administration) of from about 0.08 L to about 0.15 L. In some embodiments, administration of a pharmaceutical composition (e.g., comprising ensifentrine) to a subject having severe or very severe COPD provides a change from baseline FEV₁ to peak FEV₁ (e.g., after 12 weeks of administration) of from about 0.1 L to about 0.14 L. In some embodiments, administration of a pharmaceutical composition (e.g., comprising ensifentrine) to a subject having severe or very severe COPD provides a change from baseline FEV₁ to peak FEV₁ (e.g., after 12 weeks of administration) of about 0.11 L.

In some embodiments, ensifentrine is effective in subjects having mild, moderate, severe, or very severe COPD compared to baseline. In some instances, subjects with mild or moderate COPD experience greater changes in baseline FEV₁ to peak FEV₁. In some embodiments, the change in baseline FEV₁ to peak FEV₁ for a subject having mild or moderate COPD is at least 30% greater than for a subject having severe or very severe COPD. In some embodiments, the change in baseline to peak FEV₁ for a subject having mild or moderate COPD is at least 40% greater than for a subject having severe or very severe COPD. In some embodiments, the change in baseline to peak FEV₁ for a subject having mild or moderate COPD is from about 40% to about 60% greater than for a subject having severe of very severe COPD. In some embodiments, the change in baseline to peak FEV₁ for a subject having mild or moderate COPD is about 55% greater than for a subject having severe or very severe COPD.

In some instances, subjects with severe or very severe COPD experience greater changes in baseline FEV₁ to peak FEV₁. In some embodiments, the change in baseline FEV₁ to peak FEV₁ for a subject having severe or very severe COPD is at least 30% greater than for a subject having mild or moderate COPD. In some embodiments, the change in baseline FEV₁ to peak FEV₁ for a subject having severe or very severe COPD is at least 40% greater than for a subject having mild or moderate COPD. In some embodiments, the change in baseline FEV₁ to peak FEV₁ for a subject having severe or very severe COPD is from about 40% to about 60% greater than for a subject having mild or moderate COPD. In some embodiments, the change in baseline FEV₁ to peak FEV₁ for a subject having severe or very severe COPD is about 55% greater than for a subject having mild or moderate COPD. In some embodiments, administration of a pharmaceutical composition (e.g., comprising ensifentrine) to a subject having mild or moderate COPD provides a FEV₁ AUC_{0-4 h} (e.g., after 12 weeks of administration) of at least 0.13 L (130 mL). In some embodiments, administration of a pharmaceutical composition (e.g., comprising ensifentrine) to a subject having mild or moderate COPD provides a FEV₁ AUC_{0-4 h}(e.g., after 12 weeks of administration) of at least 0.14 L (e.g., 0.15 L, 0.16 L). In some embodiments, administration of a pharmaceutical composition (e.g., comprising ensifentrine) to a subject having mild or moderate COPD provides a FEV₁ AUC_{0-4 h} (e.g., after 12 weeks of administration) of at most 0.19 L (190 mL). In some embodiments, administration of a pharmaceutical composition (e.g., comprising ensifentrine) to a subject having mild or moderate COPD provides a FEV₁ AUC_{0-4 h} (e.g., after 12 weeks of administration) of at most 0.18 L (e.g., 0.17 L, 0.16 L). In some embodiments, administration of a pharmaceutical composition (e.g., comprising ensifentrine) to a subject having mild or moderate COPD provides a FEV₁ AUC_{0-4 h} (e.g., after 12 weeks of administration) of from about 0.13 L to about 0.19 L. In some embodiments, administration of a pharmaceutical composition (e.g., comprising ensifentrine) to a subject having mild or moderate COPD provides a FEV₁ AUC_{0-4 h} (e.g., after 12 weeks of administration) of about 0.14 L to about 0.18 L. In some embodiments, administration of a pharmaceutical composition (e.g., comprising ensifentrine) to a subject having mild or moderate COPD provides a FEV₁ AUC_{0-4 h} (e.g., after 12 weeks of administration) of about 0.16 L (160 mL).

In some embodiments, administration of a pharmaceutical composition (e.g., comprising ensifentrine) to a subject having severe or very severe COPD provides a FEV₁ AUC_{0-4 h}(e.g., after 12 weeks of administration) of at least 0.07 L (70 mL). In some embodiments, administration of a pharmaceutical composition (e.g., comprising ensifentrine) to a subject having severe or very severe COPD provides a FEV₁ AUC_{0-4 h} (e.g., after 12 weeks of administration) of at least 0.08 L (e.g., 0.09 L, 0.1 L). In some embodiments, administration of a pharmaceutical composition (e.g., comprising ensifentrine) to a subject having severe or very severe COPD provides a FEV₁ AUC_{0-4 h} (e.g., after 12 weeks of administration) of at most 0.14 L (140 mL). In some embodiments, administration of a pharmaceutical composition (e.g., comprising ensifentrine) to a subject having severe or very severe COPD provides a FEV₁ AUC_{0-4 h}(e.g., after 12 weeks of administration) of at most 0.13 L (e.g., 0.12 L, 0.11 L, 0.1 L). In some embodiments, administration of a pharmaceutical composition (e.g., comprising ensifentrine) to a subject having severe or very severe COPD provides a FEV₁ AUC_{0-4 h} (e.g., after 12 weeks of administration) of about 0.07 L to about 0.14 L. In some embodiments, administration of a pharmaceutical composition (e.g., comprising ensifentrine) to a subject having severe or very severe COPD provides a FEV₁ AUC_{0-4 h} (e.g., after 12 weeks of administration) of about 0.08 L to about 0.12 L. In some embodiments, administration of a pharmaceutical composition (e.g., comprising ensifentrine) to a subject having severe or very severe COPD provides a FEV₁ AUC_{0-4 h} (e.g., after 12 weeks of administration) of about 0.1 L (e.g., 100 mL).

In some embodiments, ensifentrine is effective in subjects having mild, moderate, severe or very severe COPD. In some instances, following treatment with a composition described herein, subjects with mild or moderate COPD experience greater changes in FEV₁ AUC_{0-4 h}. In some embodiments, the FEV₁ AUC_{0-4 h} for a subject having mild or moderate COPD is at least 40% greater than for a subject having severe or very severe COPD following treatment with a composition described herein. In some embodiments, the FEV₁ AUC_{0-4 h} for a subject having mild or moderate COPD is at most 70% greater than for a subject having severe or very severe COPD following treatment with a composition described herein. In some embodiments, the FEV₁ AUC_{0-4 h} for a subject having mild or moderate COPD is from about 50% to about 70% greater than for a subject having severe or very severe COPD following treatment with a composition described herein. In some embodiment, the FEV₁ AUC_{0-4 h} for a subject having mild or moderate COPD is about 60% greater than for a subject having severe or very severe COPD following treatment with a composition described herein.

In some instances, following treatment with a composition described herein, subjects with severe or very severe COPD experience greater changes in FEV₁ AUC_{0-4 h}. In some embodiments, the FEV₁ AUC_{0-4 h} for a subject having severe or very severe COPD is at least 40% greater than for a subject having mild or moderate COPD following treatment with a composition described herein. In some embodiments, the FEV₁ AUC_{0-4 h} for a subject having severe or very severe COPD is at least 70% greater than for a subject having mild or moderate COPD following treatment with a composition described herein. In some embodiments, the FEV₁ AUC_{0-4 h} for a subject having severe or very severe COPD is at most 70% greater than for a subject having mild or moderate COPD following treatment with a composition described herein. In some embodiments, the FEV₁ AUC_{0-4 h} for a subject having severe or very severe COPD is from about 50% to about 70% greater than for a subject having mild or moderate COPD following treatment with a composition described herein. In some embodiments, the FEV₁ AUC_{0-4 h} for a subject having severe or very severe COPD is about 60% greater than for a subject having mild or moderate COPD following treatment with a composition described herein.

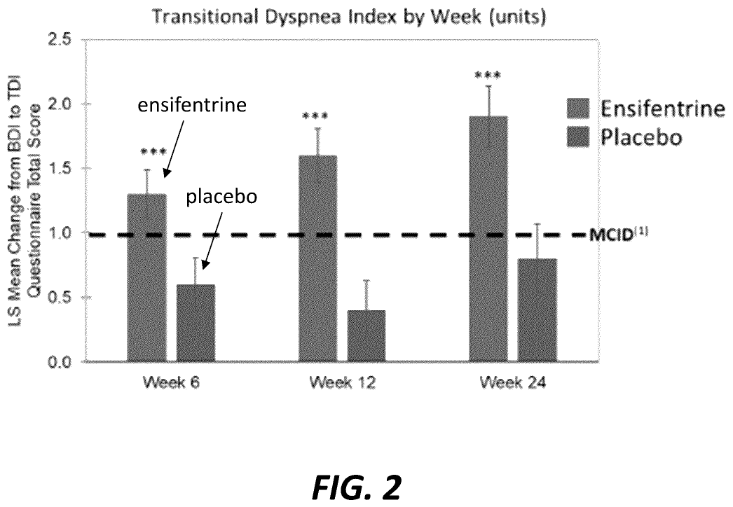

In some embodiments, administration of a pharmaceutical composition provided herein provides an increase in TDI (transition dyspnea index) score in comparison to a placebo. In some embodiments, the TDI score increases by at least 100% in comparison to a placebo. In some embodiments, the TDI score increases by at most 300% in comparison to a placebo. In some embodiments, the TDI score increases by from about 100% to about 300% in comparison to a placebo. In some embodiments, the TDI score increases by about 160% in comparison to a placebo. In some embodiments, the TDI score increases by about 275% in comparison to a placebo. In some embodiments, the TDI score increases by about 150% in comparison to a placebo. In some instances, the TDI score increases with increasing length of treatment, such as shown in FIG. 2.

FEV₁ as described herein may be measured using spirometry, which the skilled artisan would understand as a well standardized, easy to perform, and consistent reproducible measure of pulmonary function.

### Trough Lung Function

In some embodiments, provided herein are methods of increasing trough lung function in a subject suffering from COPD, the method comprising administering to the subject (e.g., a therapeutically effective amount of) ensifentrine. In some embodiments, provided herein are methods of increasing trough lung function in a subject suffering from COPD, the method comprising administering to the subject a pharmaceutical composition (e.g., comprising ensifentrine) described elsewhere herein. In some embodiments, the pharmaceutical compositions provided herein may increase trough lung function in subjects with any severity of COPD, such as mild, moderate, severe, or very severe. In some embodiments, the subject is suffering from moderate COPD. In some embodiments, provided herein is a method of increasing trough lung function in a subject suffering from moderate COPD. In some instances, ensifentrine provides benefits for subjects with mild or moderate COPD over other PDE4 inhibitors (e.g., roflumilast), which in some instances have been shown to only be effective in subjects with severe COPD.

In some instances, improving trough lung function comprises improvements in morning and night trough lung function. In some embodiments, improving trough lung function comprises improvements in morning trough lung function. In some embodiments, improving night trough lung function.

In some embodiments, provided herein is a method of treating COPD in a subject comprising administering (e.g., a therapeutically effective amount of) ensifentrine, wherein the subject is susceptible to disturbed sleep. In some embodiments, provided herein is a method of treating COPD in a subject comprising administering a pharmaceutical composition described elsewhere herein, wherein the subject is susceptible to disturbed sleep.

In some instances, an increase in trough lung function in a subject is determined by measuring an increase in trough FEV₁ (e.g., the FEV₁ of the subject shortly before administration of the compound as part of the maintenance therapy). The improvement in trough lung function may for example arise from a change in maintenance therapy drug.

In some embodiments, the methods herein comprise increasing morning trough lung function (e.g., trough lung function following sleep). Morning trough lung function may be measured by determining the FEV₁ of the subject shortly before the morning administration of the pharmaceutical composition as part of the maintenance therapy. In some embodiments, FEV₁ may be measured less than an hour before the morning administration of the ensifentrine. Morning trough FEV₁ may be the FEV₁ as measured between 11.5 and 12 h following the prior evening dose.

In some embodiments, improvement in trough lung function can improve sleep in a COPD subject. In some instances, this may be important in subjects susceptible to disturbed sleep. Subjects susceptible to disturbed sleep typically suffer from a condition that directly affects their ability to sleep (e.g., insomnia or sleep apnea) or from a condition that indirectly makes it harder to sleep (e.g., skin condition such as psoriasis that irritate the skin and make it harder for a patient to fall asleep or remain asleep).

As used herein, a “patient susceptible to disturbed sleep” is typically a patient suffering from one or more disease or condition selected from obesity, insomnia, sleep apnea, narcolepsy, restless leg syndrome, REM sleep behavior disorder, circadian rhythm sleep disorders parasomnias, depression, anxiety, psoriasis, dermatitis, eczema, or urticaria. In some embodiments, the ensifentrine may be used in treating COPD in a subject suffering from COPD and sleep apnea. In some embodiments, the compound may be used in treating COPD in a subject suffering from COPD and a skin condition such as psoriasis, dermatitis, eczema, or urticaria.

In some embodiments, the improvement in trough lung function can (e.g., significantly) assist a COPD subject in exercising. In some instances, the subject is suffering from obesity.

In some embodiments, administration of a pharmaceutical composition provided herein to a subject with moderate COPD increases trough lung function. In some embodiments, trough function may be increased after administration with ensifentrine in subjects with any level of COPD severity (e.g., mild, moderate, severe, or very severe). In some instances, subjects with moderate COPD experience large increases in trough lung function following treatment with a composition described herein. In some instances, subjects with severe COPD experience large increases in trough lung function following treatment with a composition described herein. In some instances, subjects with very severe COPD experience large increases in trough lung function following treatment with a composition described herein. In some embodiments, subjects with mild COPD experience large increases in trough lung function following treatment with a composition described herein. In some embodiments, administration of a pharmaceutical composition provided herein to a subject with moderate COPD provides a mean change in baseline to morning trough FEV₁ (forced expiratory volume in one second) of from about 0.03 L (30 mL) to about 0.09 L (90 mL). In some embodiments, administration of a pharmaceutical composition provided herein to a subject with moderate COPD provides a mean change in baseline to morning trough FEV₁ (forced expiratory volume in one second) of at least 0.03 L (30 mL). In some embodiments, administration of a pharmaceutical composition provided herein to a subject with moderate COPD provides a mean change in baseline to morning trough FEV₁ (forced expiratory volume in one second) of at least 0.04 L (e.g., 0.05 L, 0.06 L). In some embodiments, administration of a pharmaceutical composition provided herein to a subject with moderate COPD provides a mean change in baseline to morning trough FEV₁ (forced expiratory volume in one second) of at most 0.09 L (90 mL). In some embodiments, administration of a pharmaceutical composition provided herein to a subject with moderate COPD provides a mean change in baseline to morning trough FEV₁ (forced expiratory volume in one second) of at most 0.08 L (e.g., 0.07 L, 0.06 L). In some embodiments, administration of a pharmaceutical composition provided herein to a subject with moderate COPD provides a mean change in baseline to morning trough FEV₁ (forced expiratory volume in one second) of from about 0.05 L to about 0.07 L. In some embodiments, administration of a pharmaceutical composition provided herein to a subject with moderate COPD provides a mean change in baseline to morning trough FEV₁ (forced expiratory volume in one second) of about 0.06 L (60 mL). In some instances, the change in baseline to morning trough FEV₁ is measured after 12 weeks of administration of the pharmaceutical composition provided herein, such as described in Example 1.

In some embodiments, the pharmaceutical compositions provided herein increase trough lung function in subjects with any severity level of COPD. In some instances subjects with moderate COPD respond to a greater extent than subjects with other etiologies. In some embodiments, following treatment with a composition described herein, the change in baseline to morning trough FEV₁ for a subject with moderate COPD is at least 50% greater than for a subject with severe or very severe COPD. In some embodiments, following treatment with a composition described herein, the change in baseline to morning trough FEV₁ for a subject with moderate COPD is at least 75% greater than for a subject with severe or very severe COPD. In some embodiments, following treatment with a composition described herein, the change in baseline to morning trough FEV₁ for a subject with moderate COPD is about 100% greater than for a subject with severe of very severe COPD. In some embodiments, following treatment with a composition described herein, the change in baseline to morning trough FEV₁ for a subject with moderate COPD is at most 150% greater than for a subject with severe or very severe COPD.

In some instances subjects with severe or very severe COPD respond to a greater extent than subjects with other etiologies following treatment with a composition described herein. In some embodiments, following treatment with a composition described herein, the change in baseline to morning trough FEV₁ for a subject with severe or very severe COPD is at least 50% greater than for a subject with moderate COPD. In some embodiments, following treatment with a composition described herein, the change in baseline to morning trough FEV₁ for a subject with severe or very severe COPD is at least 75% greater than for a subject with moderate COPD. In some embodiments, following treatment with a composition described herein, the change in baseline to morning trough FEV₁ for a subject with severe or very severe COPD is about 100% greater than for a subject with moderate COPD. In some embodiments, following treatment with a composition described herein, the change in baseline to morning trough FEV₁ for a subject with severe or very severe COPD is at most 150% greater than for a subject with moderate COPD.

### COPD Exacerbations

In some embodiments, provided herein are methods of treating the frequency and/or severity of COPD exacerbations in a subject suffering from COPD, the method comprising administering to the subject (e.g., a therapeutically effective amount of) ensifentrine comprised in a pharmaceutical composition provided herein. In some embodiments, provided herein is a method of treating the frequency of COPD exacerbations in a subject, the method comprising administering to the subject (e.g., a therapeutically effective amount of) ensifentrine comprised in a pharmaceutical composition provided herein. In some embodiments, provided herein is a method of treating the severity of COPD exacerbations in a subject, the method comprising administering to the subject (e.g., a therapeutically effective amount of) ensifentrine comprised in a pharmaceutical composition provided herein. Also provided herein in some embodiments is a method of increasing the time to first COPD exacerbation in a subject suffering from COPD, comprising administering (e.g., a therapeutically effective amount of) ensifentrine comprised in a pharmaceutical composition provided herein. In some embodiments, provided herein are methods of treating the frequency/severity of COPD exacerbations in subjects suffering from moderate COPD and have chronic bronchitis. Ensifentrine may provide advantages in subjects suffering from moderate COPD and chronic bronchitis over other PDE4 inhibitors (e.g., roflumilast), as in some instances, other PDE4 inhibitors are only effective in subjects with severe COPD.

In some embodiments, provided herein are methods of decreasing the frequency and/or severity of COPD exacerbations in a subject. In some embodiments, ensifentrine is effective in decreasing the frequency and/or severity of COPD exacerbations regardless of the severity level of the COPD (e.g., mild, moderate, severe, or very severe). In some instances, following treatment with a composition described herein, subjects with moderate COPD experience greater decreases in frequency and/or severity of COPD exacerbations. In some instances, following treatment with a composition described herein, subjects with severe COPD experience greater decreases in frequency and/or severity of COPD exacerbations. In some embodiments, following treatment with a composition described herein, the pharmaceutical compositions decrease the frequency and/or severity of COPD exacerbations in subjects with moderate COPD. In some embodiments, the pharmaceutical compositions disclosed herein decrease the frequency and/or severity of COPD exacerbations in subjects with severe COPD. In some embodiments, the subject has moderate COPD. In some embodiments, the subject has a baseline blood eosinophils concentration of less than or equal to 150 cells/μL. In some embodiments, provided herein are methods of decreasing the frequency and/or severity of COPD exacerbations in a subject suffering from moderate COPD. In some embodiments, provided herein are methods of decreasing the frequency and/or severity of COPD exacerbations in a subject with a baseline blood eosinophils concentration of less than or equal to 150 cells/μL. Ensifentrine's ability to decrease the frequency and/or severity of COPD exacerbations in subjects with blood eosinophil concentrations of less than or equal to 150 cells/μL may be indicative of ensifentrine's effects on Type 1 inflammation in COPD patients. This is in contrast to data with the anti-inflammatory effects of ICS, which are known to be limited to COPD patients with high eosinophil levels. In some embodiments, provided herein are methods of decreasing the severity and/or frequency of COPD exacerbations in subjects suffering from moderate COPD and having a baseline blood eosinophils concentration of less than or equal to 150 cells/μL.

In some embodiments, provided herein is a method of treating COPD in a subject, comprising administering to the subject (e.g., a therapeutically effective amount of) ensifentrine or a pharmaceutically acceptable salt thereof, wherein the subject is susceptible to COPD exacerbations. In some embodiments, provided herein is a method of treating COPD in a subject, comprising administering to the subject a pharmaceutical composition provided elsewhere herein, wherein the subject is susceptible to COPD exacerbations.

In some embodiments, the pharmaceutical compositions provided herein may decrease the frequency and/or severity of COPD exacerbations in a subject suffering from COPD. In some embodiments, the pharmaceutical compositions provided herein decrease the frequency of COPD exacerbations. For example, the subject may suffer two or fewer (e.g., one or zero) COPD exacerbations per year while being treated with the pharmaceutical composition, for example as a maintenance therapy. The number of COPD exacerbations experienced by the subject per year during treatment with the pharmaceutical composition may be one to three fewer than the number of COPD exacerbations experienced by the subject per year prior to treatment with the pharmaceutical composition.

In some embodiments, administration of a pharmaceutical composition provided herein reduces the rate of COPD exacerbations by from about 30% to about 50%. In some embodiments, administration of a pharmaceutical composition provided herein reduces the rate of COPD exacerbations by from about 35% to about 45%. In some embodiments, administration of a pharmaceutical composition provided herein reduces the rate of COPD exacerbations by at least 25%. In some embodiments, administration of a pharmaceutical composition provided herein reduces the rate of COPD exacerbations by at least 30% (e.g., 32%, 34%, 36%, 38%, 40%, or 42%). In some embodiments, administration of a pharmaceutical composition provided herein reduces the rate of COPD exacerbations by at most 50% (e.g., 48%, 46%, 44%, 42%, or 40%). In some embodiments, administration of a pharmaceutical composition provided herein reduces the rate of COPD exacerbations by about 36%, 43%, or 44%. In some embodiments, administration of a pharmaceutical composition provided herein reduces the rate of COPD exacerbations by about 40%. In some embodiments, administration of a pharmaceutical composition provided herein reduces the rate of COPD exacerbations by about 36%.

In some embodiments, administration of a pharmaceutical composition provided herein may provide longer times to a first moderate or severe COPD exacerbation (e.g., such as compared to subjects administered with a placebo). In some embodiments, administration of a pharmaceutical composition provided herein may provide at least a 25% reduction in risk of a COPD exacerbation (e.g., such as over 24 weeks of treatment). In some embodiments, administration of a pharmaceutical composition provided herein may provide at least a 30% (e.g., 32%, 34%, 36%, or 38%) reduction in risk of a COPD exacerbation. In some embodiments, administration of ensifentrine may provide at most a 60% reduction in COPD risk (e.g., 58%, 56%, 52%, 50%, 46%, or 42%). In some embodiments, administration of a pharmaceutical composition provided herein may provide a reduction in COPD exacerbation risk of about 38%. In some embodiments, administration of a pharmaceutical composition provided herein may provide a reduction in COPD exacerbation risk of about 52% (e.g., such as over 48 weeks of administration). In some instances, the reduction of risk of a COPD exacerbation may be determined by Cox's proportional hazard test and/or by log-rank test.

In some embodiments, administration of a pharmaceutical composition provided herein provides reductions in moderate or severe COPD exacerbations in subjects with a baseline blood eosinophils >150 cells/μL. In some embodiments, administration of a pharmaceutical composition provided herein provides reductions in moderate or severe COPD exacerbations in subjects with a baseline blood eosinophils ≤150 cells/μL. In some instances, these reductions in COPD exacerbation may be indicative of effects of ensifentrine on both Type 1 inflammation in COPD patients (e.g., more neutrophilic) and Type 2 inflammation (e.g., more eosinophilic). In some instances, this includes significant effects on neutrophils, macrophages, lymphocytes, and eosinophils. This is in contrast to data with the anti-inflammatory effects of ICS, which are known to be limited to COPD patients with high blood eosinophils.

In some embodiments, the pharmaceutical compositions provided herein may increase the time to a first COPD exacerbation in a subject. In some instances, the subject may not have yet experienced a COPD exacerbation and the pharmaceutical compositions may increase the time until the subject experiences a first COPD exacerbation (e.g., the first COPD exacerbation is delayed). The pharmaceutical compositions may accordingly reduce the risk of COPD exacerbations in a subject with COPD.

The COPD exacerbations as described herein may comprise one or more of dyspnea (breathlessness), increased coughing, increased sputum volume, sputum purulence, wheezing, sore throat, a cold, and fever. Sputum purulence is a change in the color of spontaneously expectorated samples from uncolored to yellow-green. The COPD exacerbation may last for at least one day or at least two days. In some embodiments, the COPD exacerbation lasts for one day. In some embodiments, the COPD exacerbation lasts for at least one day. In some embodiments, the COPD exacerbation lasts for at least two days.

In some embodiments, the methods of treating the frequency and/or severity of COPD exacerbations also treat dyspnea in the subject. In some embodiments, the methods of treating the frequency and/or severity of COPD exacerbations also decrease the frequency of coughing in the subject. In some embodiments, the methods of treating the frequency and/or severity of COPD exacerbations also decrease sputum volume in the subject. In some embodiments, the methods of treating the frequency and/or severity of COPD exacerbations also improve sputum purulence in the subject. In some embodiments, the methods of treating the frequency and/or severity of COPD exacerbations also improve wheezing in the subject. In some embodiments, the methods of treating the frequency and/or severity of COPD exacerbations also improve sore throat in the subject. In some embodiments, the methods of treating the frequency and/or severity of COPD exacerbations also improve symptoms of a cold in the subject. In some embodiments, the methods of treating the frequency and/or severity of COPD exacerbations also improve symptoms of a fever in the subject.

In some embodiments, COPD exacerbation comprises worsening of two or more of the following major symptoms for at least two consecutive days: dyspnea, sputum volume and sputum purulence. In some embodiments, COPD exacerbation comprises worsening of any one major symptom together with any one of the following minor symptoms for at least two consecutive days: sore throats, colds (nasal discharge and/or nasal congestion), fever (oral temperature >37.5° C.) without other cause and increased cough. In some instances, a COPD exacerbation comprises worsening of two or more of the major symptoms (dyspnea, sputum volume, and sputum purulence) for at least two consecutive days.

A COPD exacerbation may be a moderate COPD exacerbation or a severe COPD exacerbation. A moderate exacerbation is defined as worsening symptoms of COPD (as defined hereinabove) requiring a minimum of three days of treatment with oral/systemic corticosteroids and/or antibiotics. A severe exacerbation is defined as worsening symptoms of COPD (as defined hereinabove) requiring in-patient hospitalization. In some embodiments, the pharmaceutical compositions provided herein reduce the severity of COPD exacerbations in a patient, and may be used in preventing severe COPD exacerbations in a subject. In some embodiments, pharmaceutical compositions reduce the severity of COPD exacerbations. In some embodiments, the pharmaceutical compositions reduce the severity of COPD exacerbations. In some instances, the subject may experience no severe COPD exacerbations in the year following first administration of the pharmaceutical composition.

In some embodiments, the administration of a pharmaceutical composition provided herein lengthens the time to a COPD exacerbation. In some embodiments, the pharmaceutical composition may increase the time to a COPD exacerbation by at least one week. In some embodiments, the pharmaceutical composition may increase the time to a COPD exacerbation by at least 2 weeks. In some embodiments, the pharmaceutical composition may increase the time to a COPD exacerbation by at least 1 month. In some embodiments, the pharmaceutical composition may increase the time to a COPD exacerbation by at least two months. In some embodiments, the pharmaceutical composition may increase the time to a COPD exacerbation by at least three, four, five, or six months.

A “subject susceptible to COPD exacerbations” as described herein may be a subject suffering from one or more co-morbidities (other than COPD). In some instances, said subject is suffering from one or more disease or condition selected from asthma, pulmonary hypertension, bronchiectasis, allergy, lung cancer, chest infection, cystic fibrosis, pulmonary fibrosis, pneumonia, hay fever, allergic rhinitis, bronchitis, emphysema, adult respiratory distress syndrome (ARDS), interstitial lung disease or tuberculosis, optionally wherein the asthma is allergic asthma, steroid resistant asthma, severe asthma, or pediatric asthma. The patient susceptible to COPD exacerbation may have a chronic bronchitis etiology, may have impaired lung function (such as 30 to 70% predicted FEV₁) or may have COPD symptoms despite use of long-acting muscarinic receptor antagonist (LAMA) or a long-acting beta-adrenergic receptor agonist (LABA) therapy.

In some embodiments, risk factors for COPD exacerbation include: high serum immunoglobulin (Ig), previous tuberculosis, severe airflow obstruction, chest infection and one or more hospital admission for COPD exacerbations in the previous year.

In some embodiments, the subjects provided herein may have suffered one or more COPD exacerbations in the year preceding the first administration of the pharmaceutical composition. In some embodiments, the subject may have suffered two or more COPD exacerbations in the year preceding the first administration of the pharmaceutical composition. In some embodiments, the subject may have suffered from at least one severe COPD exacerbation (e.g., requiring hospital treatment) in the preceding year. The subject may have suffered from one or more COPD exacerbations in the six months preceding the first administration of the pharmaceutical composition, or one or more COPD exacerbations in the one month preceding the first administration of the pharmaceutical composition.

In some embodiments, provided herein are methods of decreasing the frequency and/or severity of COPD exacerbations in a subject. In some embodiments, the subject is suffering from moderate COPD. In some embodiments, provided herein are methods of decreasing the frequency and/or severity of COPD exacerbations in a subject suffering from moderate COPD. In some embodiments, the subject has a baseline blood eosinophils level of less than or equal to 150 cells/μL. In some embodiments, provided herein are methods of decreasing the frequency and/or severity of COPD exacerbations in a subject suffering from moderate COPD and having a baseline blood eosinophils level of less than or equal to 150 cells/μL. The decrease in frequency and/or severity of COPD exacerbations may be measured by an amount of time to a first (e.g., moderate or severe) COPD exacerbation. In some embodiments, the amount of time to a first exacerbation is assessed via a hazard ratio from a Cox model, such as described in Example 1. In other embodiments, the decrease in frequency and/or severity of COPD exacerbations is measured by assessment of exacerbation risk, such as measured by a time to first event (as described in Example 1).

In some embodiments, the COPD exacerbations are moderate to severe COPD exacerbations. In some embodiments, the COPD exacerbations are moderate COPD exacerbations. In some embodiments, the COPD exacerbations are severe COPD exacerbations.

COPD exacerbations can be life-threatening and have been linked to comorbid conditions. They may also be associated with irreversible increases in airflow obstruction, which may lead to a higher frequency of hospitalization and increased risk of death. Exacerbations are also important events in a patient's COPD progression since they have a negative impact on health-related quality of life and may be associated with an accelerated decline in lung function and increased mortality. As such, methods of treating/decreasing the frequency of COPD exacerbations are desirable.

In some embodiments, the (e.g., moderate to severe) COPD exacerbations are decreased by at least 30% after administration of a pharmaceutical composition provided herein. In some embodiments, the (e.g., moderate to severe) COPD exacerbations are decreased by at least 40% after administration of a pharmaceutical composition provided herein. In some embodiments, the (e.g., moderate to severe) COPD exacerbations are decreased by about 36% after administration of a pharmaceutical composition provided herein. In some embodiments, the (e.g., moderate to severe) COPD exacerbations are decreased by about 44% after administration of a pharmaceutical composition provided herein. In some embodiments, the (e.g., moderate to severe) COPD exacerbations are decreased by at most 55% (e.g., 54%, 52%, 50%, 48%, or 46%) after administration of a pharmaceutical composition provided herein. In some embodiments, the decrease is measured in comparison to subjects administered a placebo.

In some embodiments, decreasing the frequency and/or severity of COPD exacerbations comprises increasing the amount of time to a first COPD exacerbation. In some embodiments, as described hereinabove, this is measured via a hazard ratio. In some embodiments, the hazard ratio for a subject having moderate COPD is at least 0.2 after administration of a pharmaceutical composition provided herein. In some embodiments, the hazard ratio for a subject having moderate COPD is from about 0.2 to about 0.6 after administration of a pharmaceutical composition provided herein. In some embodiments, the hazard ratio for a subject having moderate COPD is from 0.21 to 0.57 after administration of a pharmaceutical composition provided herein. In some embodiments, the hazard ratio for a subject having moderate COPD is at most 0.6 after administration of a pharmaceutical composition provided herein. In some embodiments, the hazard ratio for a subject having moderate COPD is about 0.35 after administration of a pharmaceutical composition provided herein.

In some embodiments, the hazard ratio for a subjecting having severe or very severe COPD is from about 0.6 to about 1.4 after administration of a pharmaceutical composition provided herein. In some embodiments, the hazard ratio for a subject having severe or very severe COPD is from 0.6 to 1.35 after administration of pharmaceutical composition provided herein. In some embodiments, the hazard ratio for a subject having severe or very severe COPD is at least 0.6 after administration of pharmaceutical composition provided herein. In some embodiments, the hazard ratio for a subject having severe or very severe COPD is at most 1.4 after administration of pharmaceutical composition provided herein. In some embodiments, the hazard ratio for a subject having severe or very severe COPD is about 0.9 after administration of pharmaceutical composition provided herein.

In some embodiments, ensifentrine is suitable for treatment of a subject with any level of COPD severity (e.g., mild, moderate, severe, or very severe). In some instances, following treatment with a composition described herein, subjects with moderate COPD may experience reduced hazard ratios in comparison to other subjects with other levels of COPD severity. In some embodiments, the hazard ratio for a subject having moderate COPD is at least 30% lower than for a subject having sever or very severe COPD, such as after administration with a pharmaceutical composition provided herein. In some embodiments, the hazard ratio for a subject having moderate COPD is at least 40% (e.g., at least 45%, 50%, 55%, or 60%) lower than for a subject having severe or very severe COPD after administration of a pharmaceutical composition provided herein. In some embodiments, the hazard ratio for a subject having moderate COPD is at most 90% lower than for a subject having severe or very severe COPD after administration of a pharmaceutical composition provided herein. In some embodiments, the hazard ratio for a subject having moderate COPD is at most 80% (e.g., at most 75%, 70%, 65% or 60%) lower than for a subject having severe or very severe COPD after administration of a pharmaceutical composition provided herein. In some embodiments, the hazard ratio for a subject having moderate COPD is about 40% to about 90% lower than for a subject having severe or very severe COPD after administration of pharmaceutical composition provided herein. In some embodiments, the hazard ratio for a subject having moderate COPD is about 50% to about 70% lower than for a subject having severe or very severe COPD after administration of a pharmaceutical composition provided herein. In some embodiments, the hazard ratio for a subject having moderate COPD is about 60% lower than for a subject having severe or very severe COPD after administration of a pharmaceutical composition provided herein.

In some instances, subjects with severe or very severe COPD may experience reduced hazard ratios in comparison to other subjects with other levels of COPD severity. In some embodiments, the hazard ratio for a subject having severe or very severe COPD is at least 30% lower than for a subject having moderate COPD after administration of a pharmaceutical composition provided herein. In some embodiments, the hazard ratio for a subject having severe or very severe COPD is at least 40% (e.g., at least 45%, 50%, 55%, or 60%) lower than for a subject having moderate COPD after administration of a pharmaceutical composition provided herein. In some embodiments, the hazard ratio for a subject having severe or very severe COPD is at most 90% lower than for a subject having moderate COPD after administration of a pharmaceutical composition provided herein. In some embodiments, the hazard ratio for a subject having severe or very severe COPD is at most 80% (e.g., at most 75%, 70%, 65%, or 60%) lower than for a subject having moderate COPD after administration of a pharmaceutical composition provided herein. In some embodiments, the hazard ratio for a subject having severe or very severe COPD is about 90% lower than for a subject having moderate COPD after administration of a pharmaceutical composition provided herein. In some embodiments, the hazard ratio for a subject having severe or very severe COPD is about 50% to about 70% lower than for a subject having moderate COPD after administration of a pharmaceutical composition provided herein. In some embodiments, the hazard ratio for a subject having severe or very severe COPD is about 60% lower than for a subject having moderate COPD after administration of a pharmaceutical composition provided herein.

In some embodiments, the methods provided herein decrease the frequency and/or severity of COPD exacerbations, such as measured by a decrease in exacerbation risk (e.g., as measured by time to first event). Ensifentrine may be suitable for decreasing exacerbation risk in a subject with any level of COPD severity (e.g., mild, moderate, severe, or very severe). In some embodiments, the pharmaceutical compositions provided herein may reduce exacerbation risk in a subject with moderate COPD. In some embodiments, the pharmaceutical compositions provided herein reduce exacerbation risk in a subject with severe COPD. In some embodiments, the pharmaceutical compositions provided herein reduce exacerbation risk in a subject with very severe COPD. In some embodiments, the pharmaceutical compositions provided herein reduce exacerbation risk in a subject with moderate COPD. In some embodiments, exacerbation risk (e.g., as measured by time to first event) is reduced by at least 30% for subjects having moderate COPD after administration of pharmaceutical composition provided herein. In some embodiments, exacerbation risk (e.g., as measured by time to first event) is reduced by at least 40% for subjects having moderate COPD after administration of pharmaceutical composition provided herein. In some embodiments, exacerbation risk (e.g., as measured by time to first event) is reduced by at least 50% for subjects having moderate COPD after administration of pharmaceutical composition provided herein. In some embodiments, exacerbation risk (e.g., as measured by time to first event) is reduced by from about 30% to about 70% for subjects having moderate COPD after administration of pharmaceutical composition provided herein. In some embodiments, exacerbation risk (e.g., as measured by time to first event) is reduced by from about 50% to about 60% for subjects having moderate COPD after administration of pharmaceutical composition provided herein.

In some embodiments, administration of pharmaceutical composition provided herein demonstrates a reduction in annualized (e.g., moderate or severe) exacerbations, such as over 24 weeks of administration with the pharmaceutical composition. In some embodiments, administration of ensifentrine provides a reduction in annualized moderate/severe COPD exacerbations of at least 30%. In some embodiments, administration of pharmaceutical composition provided herein provides a reduction in annualized moderate/severe COPD exacerbations of at least 40%. In some embodiments, administration of pharmaceutical composition provided herein provides a reduction in annualized moderate/severe COPD exacerbations of at most 50%. In some embodiments, administration of pharmaceutical composition provided herein provides a reduction in annualized moderate/severe COPD exacerbations of from about 30% to about 50%. In some embodiments, administration of pharmaceutical composition provided herein provides a reduction in annualized moderate/severe COPD exacerbations of about 40%. In some embodiments, administration of pharmaceutical composition provided herein provides a reduction in annualized moderate/severe COPD exacerbations of 36%, 43%, or 44%.

In some embodiments, an exacerbation rate ratio for a subject having moderate COPD (e.g., after 24 weeks of pharmaceutical composition administration, such as described in Example 1) is at least 0.2 (e.g., at least 0.22, 0.24, 0.26, 0.28, 0.3, or 0.32). In some embodiments, an exacerbation rate ratio for a subject having moderate COPD (e.g., after 24 weeks of pharmaceutical composition administration, such as described in Example 1) is at most 0.6 (e.g., at most 0.55, 0.5, 0.45, 0.4, or 0.35). In some embodiments, an exacerbation rate ratio for a subject having moderate COPD (e.g., after 24 weeks of pharmaceutical composition administration, such as described in Example 1) is from about 0.2 to about 0.6. In some embodiments, an exacerbation rate ratio for a subject having moderate COPD (e.g., after 24 weeks of pharmaceutical composition administration, such as described in Example 1) is from about 0.2 to about 0.4. In some embodiments, an exacerbation rate ratio for a subject having moderate COPD (e.g., after 24 weeks of pharmaceutical composition administration, such as described in Example 1) is about 0.3 (e.g., 0.34).

In some embodiments, an exacerbation rate ratio for a subject having severe or very severe COPD (e.g., after 24 weeks of pharmaceutical composition administration, such as described in Example 1) is at least 0.6. In some embodiments, an exacerbation rate ratio for a subject having severe or very severe COPD (e.g., after 24 weeks of pharmaceutical composition administration, such as described in Example 1) is at least 0.7 (e.g., at least 0.75, 0.8, 0.85, or 0.9). In some embodiments, an exacerbation rate ratio for a subject having severe or very severe COPD (e.g., after 24 weeks of pharmaceutical composition administration, such as described in Example 1) is at most 1.4 (e.g., at most 1.35, 1.3, 1.2, 1.1, 1.05, or 1). In some embodiments, an exacerbation rate ratio for a subject having severe or very severe COPD (e.g., after 24 weeks of pharmaceutical composition administration, such as described in Example 1) is from about 0.6 to about 1.4. In some embodiments, an exacerbation rate ratio for a subject having severe or very severe COPD (e.g., after 24 weeks of pharmaceutical composition administration, such as described in Example 1) is from about 0.8 to about 1. In some embodiments, an exacerbation rate ratio for a subject having severe or very severe COPD (e.g., after 24 weeks of pharmaceutical composition administration, such as described in Example 1) is about 0.9 (e.g., 0.93).

In some embodiments, the pharmaceutical compositions provided herein provide a decrease in exacerbation rate ratio in subjects with moderate COPD. In some embodiments, an exacerbation rate ratio for a subject having moderate COPD (e.g., after 24 weeks of pharmaceutical composition administration, such as described in Example 1) is at least 40% lower than for a subject with severe or very severe COPD. In some embodiments, an exacerbation rate ratio for a subject having moderate COPD (e.g., after 24 weeks of pharmaceutical composition administration, such as described in Example 1) is at least 50% (e.g., at least 55%, 60%) lower than for a subject with severe or very severe COPD (e.g., being administered the pharmaceutical composition). In some embodiments, an exacerbation rate ratio for a subject having moderate COPD (e.g., after 24 weeks of pharmaceutical composition administration, such as described in Example 1) is at most 90% (e.g., at most 85%, 80%, 75%, or 70%) lower than for a subject with severe or very severe COPD being administered the pharmaceutical composition. In some embodiments, an exacerbation rate ratio for a subject having moderate COPD (e.g., after 24 weeks of pharmaceutical composition administration, such as described in Example 1) is from about 40% to about 70% lower than for a subject with severe or very severe COPD being administered the pharmaceutical composition. In some embodiments, an exacerbation rate ratio for a subject having moderate COPD (e.g., after 24 weeks of pharmaceutical composition administration, such as described in Example 1) is about 60% lower than for a subject with severe or very severe COPD being administered the pharmaceutical composition.

In some embodiments, the pharmaceutical compositions provided herein provide a decrease in exacerbation rate ratio in subjects with severe or very severe COPD. In some embodiments, an exacerbation rate ratio for a subject having severe or very severe COPD is at least 40% lower than for a subject with moderate COPD being administered the pharmaceutical composition. In some embodiments, an exacerbation rate ratio for a subject having severe or very severe COPD is at least 50% (e.g., at least 55%, 60%) lower than for a subject with moderate COPD being administered the pharmaceutical composition. In some embodiments, an exacerbation rate ratio for a subject having severe or very severe COPD is at most 90% (e.g., at most 85%, 80%, 75%, or 70%) lower than for a subject with moderate COPD being administered the pharmaceutical composition. In some embodiments, an exacerbation rate ratio for a subject having severe or very severe COPD is about 40% to about 70% lower than for a subject with moderate COPD being administered the pharmaceutical composition. In some embodiments, an exacerbation rate ratio for a subject having severe or very severe COPD is about 60% lower than for a subject with moderate COPD being administered the pharmaceutical composition.

### COPD Reversibility Status

In some embodiments, ensifentrine is suitable for treating subjects with reversible or non-reversible statuses of COPD. In some instances, subjects with reversible COPD may respond to a greater extent than subjects with non-reversible COPD. In some instances, subjects with non-reversible COPD may respond to a greater extent than subjects with reversible COPD. Provided herein are methods of treating COPD in subjects with varying COPD reversibility statuses, comprising administering a pharmaceutical composition provided elsewhere herein to the subject. In some embodiments, provided herein is a method of treating COPD in subjects, wherein the subject has a COPD reversibility status of reversible. In some embodiments, provided herein is a method of treating COPD in subjects, wherein the reversibility status is non-reversible. In some instances, reversibility status as used herein is determined after administration of a bronchodilator (e.g., albuterol) to the subject. A “reversible” reversibility status is indicative of the subject's airflow obstruction returning to normal after administration of a bronchodilator (e.g., albuterol). A “non-reversible” reversibility status is indicative of the subject's airflow obstruction not returning to normal after administration of a bronchodilator.

In some embodiments, administration of a pharmaceutical composition provided elsewhere herein provides a change from baseline FEV₁ to peak FEV₁ (e.g., after 12 weeks of administration) in subjects with a COPD reversibility status of reversible of at least 0.12 L (120 mL). In some embodiments, administration of a pharmaceutical composition provided herein provides a change from baseline FEV₁ to peak FEV₁ (e.g., after 12 weeks of administration) in subjects with a COPD reversibility status of reversible of at least 0.13 L (e.g., 0.14 L, 0.15 L, or 0.16 L). In some embodiments, administration of a pharmaceutical composition provided herein provides a change from baseline FEV₁ to peak FEV₁ (e.g., after 12 weeks of administration) in subjects with a COPD reversibility status of reversible of at most 0.22 L (220 mL). In some embodiments, administration of a pharmaceutical composition provided herein provides a change from baseline FEV₁ to peak FEV₁ (e.g., after 12 weeks of administration) in subjects with a COPD reversibility status of reversible of at most 0.21 L (e.g., 0.2 L, 0.19 L, 0.18 L, or 0.17 L). In some embodiments, administration of a pharmaceutical composition provided herein provides a change from baseline FEV₁ to peak FEV₁ (e.g., after 12 weeks of administration) in subjects with a COPD reversibility status of reversible of from about 0.12 L to about 0.22 L. In some embodiments, administration of a pharmaceutical composition provided herein provides a change from baseline FEV₁ to peak FEV₁ (e.g., after 12 weeks of administration) in subjects with a COPD reversibility status of reversible of from about 0.14 L to about 0.19 L. In some embodiments, administration of a pharmaceutical composition provided herein provides a change from baseline FEV₁ to peak FEV₁ (e.g., after 12 weeks of administration) in subjects with a COPD reversibility status of reversible of about 0.17 L (170 mL).

In some embodiments, administration of a pharmaceutical composition provided herein provides a change from baseline FEV₁ to peak FEV₁ (e.g., after 12 weeks of administration) in subjects with a COPD reversibility status of non-reversible of at least 0.1 L (100 mL). In some embodiments, administration of a pharmaceutical composition provided herein provides a change from baseline FEV₁ to peak FEV₁ (e.g., after 12 weeks of administration) in subjects with a COPD reversibility status of non-reversible of at least 0.11 L (e.g., 0.12 L, 0.13 L). In some embodiments, administration of a pharmaceutical composition provided herein provides a change from baseline FEV₁ to peak FEV₁ (e.g., after 12 weeks of administration) in subjects with a COPD reversibility status of non-reversible of at most 0.16 L (160 mL). In some embodiments, administration of a pharmaceutical composition provided herein provides a change from baseline FEV₁ to peak FEV₁ (e.g., after 12 weeks of administration) in subjects with a COPD reversibility status of non-reversible of at most 0.15 L (e.g., 0.14 L, 0.13 L). In some embodiments, administration of a pharmaceutical composition provided herein provides a change from baseline FEV₁ to peak FEV₁ (e.g., after 12 weeks of administration) in subjects with a COPD reversibility status of non-reversible of from about 0.1 L to about 0.16 L. In some embodiments, administration of a pharmaceutical composition provided herein provides a change from baseline FEV₁ to peak FEV₁ (e.g., after 12 weeks of administration) in subjects with a COPD reversibility status of non-reversible of from about 0.11 L to about 0.15 L. In some embodiments, administration of a pharmaceutical composition provided herein provides a change from baseline FEV₁ to peak FEV₁ (e.g., after 12 weeks of administration) in subjects with a COPD reversibility status of non-reversible of about 0.13 L (130 mL).

In some embodiments, ensifentrine is suitable for treating subjects with reversible or non-reversible statuses of COPD. In some instances, following treatment with a composition described herein, subjects with reversible COPD may experience greater changes in baseline FEV₁ to peak FEV₁. In some embodiments, administration of a pharmaceutical composition provided herein provides a change from baseline FEV₁ to peak FEV₁ (e.g., after 12 weeks of administration) for a subject having a reversible status of COPD at least 25% greater than for a subject having a non-reversible status of COPD. In some embodiments, administration of a pharmaceutical composition provided herein provides a change from baseline FEV₁ to peak FEV₁ (e.g., after 12 weeks of administration) for a subject having a reversible status of COPD at least 30% greater than for a subject having a non-reversible status of COPD. In some embodiments, administration of a pharmaceutical composition provided herein provides a change from baseline FEV₁ to peak FEV₁ (e.g., after 12 weeks of administration) for a subject having a reversible status of COPD about 25% greater than for a subject having a non-reversible status of COPD.

In some instances, following treatment with a composition described herein, subjects with non-reversible COPD may experience greater changes in baseline FEV₁ to peak FEV₁. In some embodiments, administration of a pharmaceutical composition provided herein provides a change in baseline FEV₁ to peak FEV₁ for a subject having a non-reversible status of COPD of at least 25% greater than for a subject having a reversible status of COPD. In some embodiments, administration of a pharmaceutical composition provided herein provides a change in baseline FEV₁ to peak FEV₁ for a subject having a non-reversible status of COPD of at least 30% greater than for a subject having a reversible status of COPD. In some embodiments, administration of a pharmaceutical composition provided herein provides a change in baseline FEV₁ to peak FEV₁ for a subject having a non-reversible status of COPD of about 25% greater than for a subject having a reversible status of COPD.

In some embodiments, administration of a pharmaceutical composition provided herein provides a FEV₁ AUC_{0-4 h} (e.g., after 12 weeks of administration) in subjects with a COPD reversibility status of reversible of at least 0.12 L (e.g., 120 mL). In some embodiments, administration of a pharmaceutical composition provided herein provides a FEV₁ AUC_{0-4 h} (e.g., after 12 weeks of administration) in subjects with a COPD reversibility status of reversible of at least 0.13 L (e.g., 0.14 L, 0.15 L, or 0.16 L). In some embodiments, administration of a pharmaceutical composition provided herein provides a FEV₁ AUC_{0-4 h} (e.g., after 12 weeks of administration) in subjects with a COPD reversibility status of reversible of at most 0.21 L (210 mL). In some embodiments, administration of a pharmaceutical composition provided herein provides a FEV₁ AUC_{0-4 h} (e.g., after 12 weeks of administration) in subjects with a COPD reversibility status of reversible of at most 0.20 L (e.g., 0.19 L, 0.18 L, 0.17 L, or 0.16 L). In some embodiments, administration of a pharmaceutical composition provided herein provides a FEV₁ AUC_{0-4 h} (e.g., after 12 weeks of administration) in subjects with a COPD reversibility status of reversible of from about 0.12 L to about 0.21 L. In some embodiments, administration of a pharmaceutical composition provided herein provides a FEV₁ AUC_{0-4 h} (e.g., after 12 weeks of administration) in subjects with a COPD reversibility status of reversible of from about 0.14 L to about 0.18 L. In some embodiments, administration of a pharmaceutical composition provided herein provides a FEV₁ AUC_{0-4 h} (e.g., after 12 weeks of administration) in subjects with a COPD reversibility status of reversible of about 0.16 L (160 mL).

In some embodiments, administration of a pharmaceutical composition provided herein provides a FEV₁ AUC_{0-4 h} (e.g., after 12 weeks of administration) in subjects with a COPD reversibility status of non-reversible of at least 0.1 L. In some embodiments, administration of a pharmaceutical composition provided herein provides a FEV₁ AUC_{0-4 h} (e.g., after 12 weeks of administration) in subjects with a COPD reversibility status of non-reversible of at least 0.11 L or 0.12 L. In some embodiments, administration of a pharmaceutical composition provided herein provides a FEV₁ AUC_{0-4 h} (e.g., after 12 weeks of administration) in subjects with a COPD reversibility status of non-reversible of at most 0.15 L (150 mL). In some embodiments, administration of a pharmaceutical composition provided herein provides a FEV₁ AUC_{0-4 h} (e.g., after 12 weeks of administration) in subjects with a COPD reversibility status of non-reversible of at most 0.14 L (e.g., 0.13 L, 0.12 L). In some embodiments, administration of a pharmaceutical composition provided herein provides a FEV₁ AUC_{0-4 h} (e.g., after 12 weeks of administration) in subjects with a COPD reversibility status of non-reversible of from about 0.1 L to about 0.15 L. In some embodiments, administration of a pharmaceutical composition provided herein provides a FEV₁ AUC_{0-4 h} (e.g., after 12 weeks of administration) in subjects with a COPD reversibility status of non-reversible of about 0.12 L (120 mL).

In some embodiments, ensifentrine is suitable for treating subjects with reversible or non-reversible statuses of COPD. In some instances, following treatment with a composition described herein, subjects with reversible COPD may experience greater changes in FEV₁ AUC_{0-4 h}. In some embodiments, administration of a pharmaceutical composition provided herein provides a FEV₁ AUC_{0-4 h} (e.g., after 12 weeks of administration) in subjects with a COPD reversibility status of reversible at least 25% greater than for a subject having a non-reversible COPD status. In some embodiments, a pharmaceutical composition provided herein provides a FEV₁ AUC_{0-4 h} (e.g., after 12 weeks of administration) in subjects with a COPD reversibility status of reversible at least 30% greater than for a subject having a non-reversible COPD status. In some embodiments, a pharmaceutical composition provided herein provides a FEV₁ AUC_{0-4 h}(e.g., after 12 weeks of administration) in subjects with a COPD reversibility status of reversible of from about 30% to about 40% greater than for a subject having a non-reversible COPD status. In some embodiments, a pharmaceutical composition provided herein provides a FEV₁ AUC_{0-4 h} (e.g., after 12 weeks of administration) in subjects with a COPD reversibility status of reversible about 33% greater than for a subject having a non-reversible COPD status.

In some instances, following treatment with a composition described herein, subjects with non-reversible COPD may experience greater changes in FEV₁ AUC_{0-4 h}. In some embodiments, administration of a pharmaceutical composition provided herein provides a FEV₁ AUC_{0-4 h} in subjects with a COPD reversibility status of non-reversible at least 25% greater than for a subject having a reversible COPD status. In some embodiments, administration of a pharmaceutical composition provided herein provides a FEV₁ AUC_{0-4 h} in subjects with a COPD reversibility status of non-reversible at least 30% greater than for a subject having a reversible COPD status. In some embodiments, administration of a pharmaceutical composition provided herein provides a FEV₁ AUC_{0-4 h} in subjects with a COPD reversibility status of non-reversible of from about 30% to about 40% greater than for a subject having a reversible COPD status. In some embodiments, administration of a pharmaceutical composition provided herein provides a FEV₁ AUC_{0-4 h} in subjects with a COPD reversibility status of non-reversible about 33% greater than for a subject having a non-reversible COPD status.

### Chronic Bronchitis

In some embodiments, provided herein are methods of treating COPD in a subject with or without chronic bronchitis. In some embodiments, provided herein are methods of treating COPD in subjects not having chronic bronchitis. In some embodiments, ensifentrine is suitable for treating subjects with or without chronic bronchitis. In some instances, subjects without chronic bronchitis respond to a greater extent to ensifentrine. In some instances, subjects with chronic bronchitis respond to a greater extent to ensifentrine. In some instances, ensifentrine provides a benefit of being effective in both subjects with and without chronic bronchitis, contrary to other PDE4 inhibitors (e.g., roflumilast) which in some instances have been shown to be effective only in subjects with chronic bronchitis.

In some embodiments, administration of a pharmaceutical composition provided herein to a subject not having chronic bronchitis provides a mean change in baseline to peak FEV₁ of from about 0.13 L to about 0.21 L. In some embodiments, administration of a pharmaceutical composition provided herein to a subject not having chronic bronchitis provides a mean change in baseline to peak FEV₁ of at least 0.13 L. In some embodiments, administration of a pharmaceutical composition provided herein to a subject not having chronic bronchitis provides a mean change in baseline to peak FEV₁ of at least 0.14 L (e.g., 0.15, 0.16, or 0.17). In some embodiments, administration of a pharmaceutical composition provided herein to a subject not having chronic bronchitis provides a mean change in baseline to peak FEV₁ of at most 0.21 L. In some embodiments, administration of a pharmaceutical composition provided herein to a subject not having chronic bronchitis provides a mean change in baseline to peak FEV₁ of at most 0.2 L (e.g., 0.19, 0.18, or 0.17). In some embodiments, administration of a pharmaceutical composition provided herein to a subject not having chronic bronchitis provides a mean change in baseline to peak FEV₁ of from about 0.13 L to about 0.21 L. In some embodiments, administration of a pharmaceutical composition provided herein to a subject not having chronic bronchitis provides a mean change in baseline to peak FEV₁ of from about 0.1 L to about 0.2 L. In some embodiments, administration of a pharmaceutical composition provided herein to a subject not having chronic bronchitis provides a mean change in baseline to peak FEV₁ of from about 0.15 L to about 0.18 L. In some embodiments, administration of a pharmaceutical composition provided herein to a subject not having chronic bronchitis provides a mean change in baseline to peak FEV₁ of about 0.17 L.

In some embodiments, administration of a pharmaceutical composition provided herein to a subject having chronic bronchitis provides a mean change in baseline FEV₁ to peak FEV₁ of at least 0.11 L. In some embodiments, administration of a pharmaceutical composition provided herein to a subject not having chronic bronchitis provides a mean change in baseline to peak FEV₁ of at least 0.1 L (e.g., 0.12 L, 0.13 L, or 0.14 L). In some embodiments, administration of a pharmaceutical composition provided herein to a subject not having chronic bronchitis provides a mean change in baseline to peak FEV₁ of at most 0.17 L. In some embodiments, administration of a pharmaceutical composition provided herein to a subject not having chronic bronchitis provides a mean change in baseline to peak FEV₁ of at most 0.16 L (e.g., 0.15 or 0.14). In some embodiments, administration of a pharmaceutical composition provided herein to a subject not having chronic bronchitis provides a mean change in baseline to peak FEV₁ of from about 0.11 L to about 0.17 L. In some embodiments, administration of a pharmaceutical composition provided herein to a subject not having chronic bronchitis provides a mean change in baseline to peak FEV₁ of from about 0.13 L to about 0.15 L. In some embodiments, administration of a pharmaceutical composition provided herein to a subject not having chronic bronchitis provides a mean change in baseline to peak FEV₁ of about 0.14 L.

In some embodiments, ensifentrine is suitable for treatment of subjects regardless of a chronic bronchitis diagnoses. In some instances, following treatment with a composition described herein, subjects without chronic bronchitis experience greater changes in baseline to peak FEV₁. In some embodiments, administration of a pharmaceutical composition provided herein provides a mean change in baseline to peak FEV₁ of at least 15% greater than that for a subject who does have chronic bronchitis. In some embodiments, administration of a pharmaceutical composition provided herein provides a mean change in baseline to peak FEV₁ of at least 20% greater than that for a subject who does have chronic bronchitis. In some embodiments, administration of a pharmaceutical composition provided herein provides a mean change in baseline to peak FEV₁ of at most 40% greater than that for a subject who does have chronic bronchitis. In some embodiments, administration of a pharmaceutical composition provided herein provides a mean change in baseline to peak FEV₁ of at most 30% greater than that for a subject who does have chronic bronchitis. In some embodiments, administration of a pharmaceutical composition provided herein provides a mean change in baseline to peak FEV₁ of about 20% (e.g., 21%) greater than that for a subject who does have chronic bronchitis.

In some instances, subjects with chronic bronchitis experience greater changes in baseline to peak FEV₁. In some embodiments, administration of a pharmaceutical composition provided herein provides a mean change in baseline to peak FEV₁ of at least 15% greater than that for a subject who does not have chronic bronchitis. In some embodiments, administration of a pharmaceutical composition provided herein provides a mean change in baseline to peak FEV₁ of at least 20% greater than that for a subject who does not have chronic bronchitis. In some embodiments, administration of a pharmaceutical composition provided herein provides a mean change in baseline to peak FEV₁ of at most 40% greater than that for a subject who does not have chronic bronchitis. In some embodiments, administration of a pharmaceutical composition provided herein provides a mean change in baseline to peak FEV₁ of at most 30% greater than that for a subject who does not have chronic bronchitis. In some embodiments, administration of a pharmaceutical composition provided herein provides a mean change in baseline to peak FEV₁ of about 20% greater than that for a subject who does not have chronic bronchitis.

In some embodiments, administration of a pharmaceutical composition provided herein provides a mean FEV₁ AUC_{0-4 h} for a subject not having chronic bronchitis that is higher than a subject with chronic bronchitis. In some embodiments, administration of a pharmaceutical composition provided herein provides a mean FEV₁ AUC_{0-4 h} for a subject not having chronic bronchitis of at least 0.11 L. In some embodiments, administration of a pharmaceutical composition provided herein provides a mean FEV₁ AUC_{0-4 h} for a subject not having chronic bronchitis of at least 0.12 L (e.g., 0.13, 0.14, or 0.15, or 0.16). In some embodiments, administration of a pharmaceutical composition provided herein provides a mean FEV₁ AUC_{0-4 h} for a subject not having chronic bronchitis of at most 0.2 L. In some embodiments, administration of a pharmaceutical composition provided herein provides a mean FEV₁ AUC_{0-4 h} for a subject not having chronic bronchitis of at most 0.19 L (e.g., 0.18, 0.17, or 0.16). In some embodiments, administration of a pharmaceutical composition provided herein provides a mean FEV₁ AUC_{0-4 h} for a subject not having chronic bronchitis of from about 0.11 L to about 0.2 L. In some embodiments, administration of a pharmaceutical composition provided herein provides a mean FEV₁ AUC_{0-4 h} for a subject not having chronic bronchitis of from about 0.14 L to about 0.18 L. In some embodiments, administration of a pharmaceutical composition provided herein provides a mean FEV₁ AUC_{0-4 h} for a subject not having chronic bronchitis of about 0.16 L.

In some embodiments, administration of a pharmaceutical composition provided herein provides a mean FEV₁ AUC_{0-4 h} for a subject having chronic bronchitis of at least 0.1 L. In some embodiments, administration of a pharmaceutical composition provided herein provides a mean FEV₁ AUC_{0-4 h} for a subject having chronic bronchitis of at least 0.11 L (e.g., 0.12 or 0.13). In some embodiments, administration of a pharmaceutical composition provided herein provides a mean FEV₁ AUC_{0-4 h} for a subject having chronic bronchitis of at most 0.16 L. In some embodiments, administration of a pharmaceutical composition provided herein provides a mean FEV₁ AUC_{0-4 h} for a subject having chronic bronchitis of at most 0.15 L (e.g., 0.14 or 0.13). In some embodiments, administration of a pharmaceutical composition provided herein provides a mean FEV₁ AUC_{0-4 h} for a subject having chronic bronchitis of from about 0.1 L to about 0.16 L. In some embodiments, administration of a pharmaceutical composition provided herein provides a mean FEV₁ AUC_{0-4 h} for a subject having chronic bronchitis of from about 0.11 L to about 0.14 L. In some embodiments, administration of a pharmaceutical composition provided herein provides a mean FEV₁ AUC_{0-4 h} for a subject having chronic bronchitis of about 0.13 L.

In some embodiments, ensifentrine is suitable for treatment of subjects regardless of a chronic bronchitis diagnoses. In some instances, subjects without chronic bronchitis experience greater changes in FEV₁ AUC_{0-4 h}. In some embodiments, administration of a pharmaceutical composition provided herein provides a mean FEV₁ AUC_{0-4 h} for a subject not having chronic bronchitis that is at least 20% greater than for a subject having chronic bronchitis. In some embodiments, administration of a pharmaceutical composition provided herein provides a mean FEV₁ AUC_{0-4 h} for a subject not having chronic bronchitis that is at most 40% (e.g., 35%, 30%, or 25%) greater than for a subject having chronic bronchitis. In some embodiments, administration of a pharmaceutical composition provided herein provides a mean FEV₁ AUC_{0-4 h} for a subject not having chronic bronchitis that is about 23% greater than for a subject having chronic bronchitis.

In some embodiments, administration of a pharmaceutical composition provided herein provides a mean FEV₁ AUC_{0-4 h} for a subject having chronic bronchitis that is at least 20% greater than for a subject not having chronic bronchitis. In some embodiments, administration of a pharmaceutical composition provided herein provides a mean FEV₁ AUC_{0-4 h} for a subject having chronic bronchitis that is at most 40% (e.g., 35%, 30%, or 25%) greater than for a subject not having chronic bronchitis. In some embodiments, administration of a pharmaceutical composition provided herein provides a mean FEV₁ AUC_{0-4 h} for a subject having chronic bronchitis that is about 23% greater than for a subject not having chronic bronchitis.

In some embodiments, administration of a pharmaceutical composition provided herein provides a mean FEV₁ AUC_{0-12 h} for a subject not having chronic bronchitis that is higher than a subject with chronic bronchitis. In some embodiments, administration of a pharmaceutical composition provided herein provides a mean FEV₁ AUC_{0-12 h} for a subject not having chronic bronchitis of at least 0.08 L. In some embodiments, administration of a pharmaceutical composition provided herein provides a mean FEV₁ AUC_{0-12 h} for a subject not having chronic bronchitis of at least 0.09 L (e.g., 0.1, 0.11, or 0.12). In some embodiments, administration of a pharmaceutical composition provided herein provides a mean FEV₁ AUC_{0-12 h} for a subject not having chronic bronchitis of at most 0.17 L. In some embodiments, administration of a pharmaceutical composition provided herein provides a mean FEV₁ AUC_{0-12 h} for a subject not having chronic bronchitis of at most 0.16 L (e.g., 0.15, 0.14, 0.13, or 0.12). In some embodiments, administration of a pharmaceutical composition provided herein provides a mean FEV₁ AUC_{0-12 h} for a subject not having chronic bronchitis of about 0.08 L to about 0.17 L. In some embodiments, administration of a pharmaceutical composition provided herein provides a mean FEV₁ AUC_{0-12 h} for a subject not having chronic bronchitis of about 0.1 L to about 0.14 L. In some embodiments, administration of a pharmaceutical composition provided herein provides a mean FEV₁ AUC_{0-12 h} for a subject not having chronic bronchitis of about 0.12 L.

In some embodiments, administration of a pharmaceutical composition provided herein provides a mean FEV₁ AUC_{0-12 h} for a subject having chronic bronchitis of at least 0.05 L. In some embodiments, administration of a pharmaceutical composition provided herein provides a mean FEV₁ AUC_{0-12 h} for a subject having chronic bronchitis of at least 0.06 L (e.g., 0.07 or 0.08). In some embodiments, administration of a pharmaceutical composition provided herein provides a mean FEV₁ AUC_{0-12 h} for a subject having chronic bronchitis of at most 0.1 L. In some embodiments, administration of a pharmaceutical composition provided herein provides a mean FEV₁ AUC_{0-12 h} for a subject having chronic bronchitis of at most 0.09 L (e.g., 0.08). In some embodiments, administration of a pharmaceutical composition provided herein provides a mean FEV₁ AUC_{0-12 h} for a subject having chronic bronchitis of from about 0.05 L to about 0.1 L. In some embodiments, administration of a pharmaceutical composition provided herein provides a mean FEV₁ AUC_{0-12 h} for a subject having chronic bronchitis of from about 0.07 L to about 0.09 L. In some embodiments, administration of a pharmaceutical composition provided herein provides a mean FEV₁ AUC_{0-12 h} for a subject having chronic bronchitis of about 0.08 L.

In some embodiments, ensifentrine is suitable for treatment of subjects regardless of a chronic bronchitis diagnoses. In some instances, subjects without chronic bronchitis experience greater changes in FEV AUC_{0-12 h}. In some embodiments, administration of a pharmaceutical composition provided herein provides a FEV₁ AUC_{0-12 h} that is at least 30% greater for a subject not having chronic bronchitis than a subject having chronic bronchitis. In some embodiments, administration of a pharmaceutical composition provided herein provides a FEV₁ AUC_{0-12 h} that is at least 40% greater for a subject not having chronic bronchitis than a subject having chronic bronchitis. In some embodiments, administration of a pharmaceutical composition provided herein provides a FEV₁ AUC_{0-12 h} that is at least 50% greater for a subject not having chronic bronchitis than a subject having chronic bronchitis. In some embodiments, administration of a pharmaceutical composition provided herein provides a FEV₁ AUC_{0-12 h} that is at most 70% greater for a subject not having chronic bronchitis than a subject having chronic bronchitis. In some embodiments, administration of a pharmaceutical composition provided herein provides a FEV₁ AUC_{0-12 h} that is at most 60% greater for a subject not having chronic bronchitis than a subject having chronic bronchitis. In some embodiments, administration of a pharmaceutical composition provided herein provides a FEV₁ AUC_{0-12 h} that is about 50% greater for a subject not having chronic bronchitis than a subject having chronic bronchitis.

In some instances, subjects without chronic bronchitis experience greater changes in FEV AUC_{0-12 h}. In some embodiments, administration of a pharmaceutical composition provided herein provides a FEV₁ AUC_{0-12 h} that is at least 30% greater for a subject having chronic bronchitis than a subject not having chronic bronchitis. In some embodiments, administration of a pharmaceutical composition provided herein provides a FEV₁ AUC_{0-12 h} that is at least 40% greater for a subject having chronic bronchitis than a subject not having chronic bronchitis. In some embodiments, administration of a pharmaceutical composition provided herein provides a FEV₁ AUC_{0-12 h} that is at least 50% greater for a subject having chronic bronchitis than a subject not having chronic bronchitis. In some embodiments, administration of a pharmaceutical composition provided herein provides a FEV₁ AUC_{0-12 h} that is at most 70% greater for a subject having chronic bronchitis than a subject not having chronic bronchitis. In some embodiments, administration of a pharmaceutical composition provided herein provides a FEV₁ AUC_{0-12 h} that is at most 60% greater for a subject having chronic bronchitis than a subject not having chronic bronchitis. In some embodiments, administration of a pharmaceutical composition provided herein provides a FEV₁ AUC_{0-12 h} that is about 50% greater for a subject having chronic bronchitis than a subject not having chronic bronchitis.

### Inhalable Corticosteroids

Also provided herein are methods of treating COPD in subjects in the absence of administration with inhalable corticosteroids (ICS). In some embodiments, provided herein are methods of treating COPD in subjects also being administered inhalable corticosteroids (ICS). In some embodiments, ensifentrine is suitable for treatment of COPD in subjects being administered or not being administered ICS. In some instances, subjects in the absence of ICS administration may respond to an increased extent to ensifentrine. In some instances, subjects being administered ICS may respond to an increased extent to ensifentrine. In some embodiments, the method comprises improving dyspnea in the subject.

In some embodiments, the pharmaceutical compositions provided herein are effective in improving dyspnea in subjects being administered ICS and/or a LABA. In some embodiments, the pharmaceutical compositions provided herein are effective in improving dyspnea in subjects being administered ICS and being administered a long acting beta agonist (LABA). In some embodiments, the long acting beta agonist is a long-acting beta-adrenergic receptor agonist. In some embodiments, dyspnea is measured using the transition dyspnea index (TDI). In some embodiments, the TDI of a subject being administered a pharmaceutical composition provided herein is at least about 1 (e.g., at least 1.1, 1.15, 1.2, 1.25, or 1.3), while being administered both ICS and LABA. In some embodiments, the TDI of a subject being administered a pharmaceutical composition provided herein is at most 2 (e.g., 1.9, 1.8, 1.7, 1.6, 1.5, or 1.4) while being administered both ICS and LABA. In some embodiments, the TDI of a subject being administered a pharmaceutical composition provided herein is about 1 to about 1.7 (e.g., about 1.1 to about 1.6, about 1.2 to about 1.5, or about 1.3 to about 1.4).

In some embodiments, the TDI of a subject being administered both ICS and LABA is at least 50% (e.g., at least 60%, 70%, 80%, 90%, 100%, or 110%) greater for a subject being administered the pharmaceutical composition than a subject being administered a placebo. In some embodiments, the TDI of a subject being administered both ICS and LABA is at most 200% (e.g., at most 190%, 180%, 170%, 160%, 150%, or 140%) greater for a subject being administered the pharmaceutical composition than a subject being administered a placebo. In some embodiments, the TDI of a subject being administered both ICS and LABA is about 80% to about 150% (e.g., about 90% to about 140%, about 100% to about 130% or about 110% to about 130%) greater for a subject being administered the pharmaceutical composition than a subject being administered a placebo. In some embodiments, the TDI of a subject being administered both ICS and LABA is about 125% greater for a subject being administered the pharmaceutical composition than a subject being administered a placebo.

In some embodiments, the ensifentrine or pharmaceutically acceptable salt thereof may be used to treat COPD in a subject suffering from both COPD and asthma. In some embodiments, the ensifentrine or pharmaceutically acceptable salt thereof may be used in treating a subject suffering from both COPD and pulmonary hypertension. In some embodiments, the ensifentrine or pharmaceutically acceptable salt thereof may be used in treating a subject suffering from both COPD and bronchiectasis. In some embodiments, the pharmaceutical composition provided herein may be used to treat COPD in a subject suffering from both COPD and asthma. In some embodiments, the pharmaceutical composition provided herein may be used in treating a subject suffering from both COPD and pulmonary hypertension. In some embodiments, the pharmaceutical composition provided herein may be used in treating a subject suffering from both COPD and bronchiectasis.

### Administration Methods

In some embodiments, the methods provided herein comprise administering to the subject a therapeutically effective amount of ensifentrine. In some embodiments, a therapeutically effective amount of ensifentrine comprises a dose of ensifentrine as described elsewhere herein. In some embodiments, the therapeutically effective amount of ensifentrine is comprised in a pharmaceutical composition provided herein.

In some embodiments, the methods provided herein comprise administering the pharmaceutical composition to the subject by inhalation. In some embodiments, a pharmaceutical composition comprising ensifentrine and one or more acceptable excipients or diluents is administered to a subject by inhalation. In some embodiments, administration by inhalation is completed using a nebulizer, pressurized metered dose inhaler (pMDI), or dry powder inhaler (DPI). In some embodiments, administration by inhalation is completed using a nebulizer. In some embodiments, administration by inhalation is completed using a pressurized metered dose inhaler (pMDI). In some embodiments, administration by inhalation is completed using a dry powder inhaler (DPI).

In some embodiments, the methods provided herein comprise administering the pharmaceutical composition by inhalation from a nebulizer. Nebulizers aerosolize a liquid pharmaceutical composition into an aerosol that is inhaled into a patient's respiratory tract. Non-limiting examples of nebulizers include a soft mist nebuliser, a vibrating mesh nebuliser, a jet nebuliser and an ultrasonic wave nebulizer. Suitable nebuliser devices include the Philips I-neb™ (Philips), the Philips SideStream (Philips), the AeroNeb® (Philips), the Philips InnoSpire Go (Philips), the Pari LC Sprint (Pari GmbH), the AERxR™ Pulmonary Delivery System (Aradigm Corp) and the Pari LC Plus Reusable Nebuliser (Pari GmbH). The nebulizer may for instance be a PARI LC Sprint jet nebulizer with a PARI Vios® PRO Aerosol Delivery System PARI BOY© compressor. The compound may be inhaled via the nebulizer for from 1 to 15 minutes. The pharmaceutical composition may be inhaled via the nebulizer for from about 1 to about 15 minutes. In some embodiments, the pharmaceutical composition may be inhaled for at least 1 minute. In some embodiments, the pharmaceutical composition may be inhaled for about 1 minute to about 12 minutes, about 1 minute to about 8 minutes, about 1 minute to about 4 minutes, about 3 minutes to about 15 minutes, about 4 minutes to about 10 minutes, or about 6 minutes to about 12 minutes. In some embodiments, the pharmaceutical composition may be inhaled for from 5 to 7 minutes. In some embodiments, the pharmaceutical composition may be inhaled for 5 minutes. In some embodiments, the pharmaceutical composition may be inhaled for 6 minutes. In some embodiments, the pharmaceutical composition may be inhaled for 7 minutes.

### Dosing Regimens

The methods provided herein comprise administering the pharmaceutical composition to the subject at any frequency necessary to achieve therapeutic efficacy, as determined by the skilled artisan. In some embodiments, the pharmaceutical composition comprising ensifentrine is provided to the subject as a maintenance therapy. In some embodiments, the methods provided herein comprise administering the pharmaceutical composition to the subject once, twice, or three times per day, for instance twice or three times per day. In some embodiments, the methods comprise administering the pharmaceutical composition to the subject once per day. In some embodiments, the methods provided herein comprise administering the pharmaceutical composition to the subject twice per day (e.g., as a twice daily maintenance therapy). In some embodiments, the methods comprise administering the pharmaceutical composition to the subject three times per day. In some embodiments, the methods comprise administering the ensifentrine to the subject at least once per day. In some embodiments, the methods comprise administering a first dose of the pharmaceutical composition in the morning (e.g., within 3 h following waking) and a second dose of the pharmaceutical composition in the evening (e.g., within 3 h before bed). In some embodiments, morning and evening doses are administered from about 10 to about 14 h apart. In some embodiments, morning and evening doses are administered about 12 h apart.

The ensifentrine provided herein may be administered in any suitable therapeutically effective amount. In some embodiments, a daily dose of the ensifentrine is about 0.1 mg to about 20 mg. In some embodiments, the methods herein comprise administering a total daily dose of the ensifentrine of about 0.5 mg to about 10 mg. In certain embodiments, the total daily dose of the ensifentrine (e.g., ensifentrine free base) is about 5 mg to about 7 mg. In some embodiments, the total daily dose of the ensifentrine is about 6 mg per day. In some instances, the total daily dose of the ensifentrine may be 6.0 mg.

The methods provided herein may comprise administering the ensifentrine (e.g., or a pharmaceutical composition comprising ensifentrine) twice a day in two separate doses, which are the same or similar. In some instances, the method may comprise administering the compound to the subject twice a day in a first dose of from about 1 mg to about 5 mg and a second dose of from about 1 mg to about 5 mg. In certain embodiments, the method comprises administering the ensifentrine (e.g., or a pharmaceutical composition comprising ensifentrine) to the subject twice per day in a first dose of from about 2 mg to about 4 mg and a second dose of from about 2 mg to about 4 mg. In certain embodiments, the method comprises administering the ensifentrine (e.g., or a pharmaceutical composition comprising ensifentrine) to the subject twice per day in a first dose of about 3 mg and a second dose of about 3 mg.

In certain embodiments, the methods provided herein comprise administering two doses of about 3 mg of ensifentrine (e.g., or a pharmaceutical composition comprising ensifentrine) to the subject per day by inhalation. The method may comprise administering a dose of about 3 mg of the ensifentrine (e.g., or a pharmaceutical composition comprising ensifentrine) to the subject twice a day (e.g., 3 mg B.I.D.) by inhalation. In some embodiments, the method comprises administering by nebulizer a dose of about 3 mg of the ensifentrine (e.g., or a pharmaceutical composition comprising ensifentrine) to the subject twice per day. In some instances, each dose may comprise 3.0 mg of ensifentrine free base administered by nebulizer.

In some embodiments, the ensifentrine (e.g., or a pharmaceutical composition comprising ensifentrine) as provided herein is used as a maintenance therapy. In some embodiments, the methods provided herein comprise administering the pharmaceutical compositions to the subject at least once per day for at least 8 weeks. In some embodiments, the pharmaceutical compositions may be administered to the subject at least once per day for at least 16 weeks. In some embodiments, the pharmaceutical compositions may be administered to the subject at least once per day for at least 24 weeks. In some embodiments, the pharmaceutical compositions may be administered daily to the subject for at least 1 year. The methods provided herein may comprise administering the pharmaceutical compositions to the subject at least once every 24 h. In some instances, the method comprises administering the pharmaceutical compositions at least twice every 24 h, such as for at least 8 weeks, 16 weeks, or 24 weeks. In some embodiments, the pharmaceutical compositions is administered to the subject for any suitable period of time as determined by a skilled artisan.

In some embodiments, the ensifentrine is administered as a suspension formulation. In some embodiments, the ensifentrine is delivered as a dry powder, for instance a dry powder comprising particles comprising the ensifentrine and particles of a carrier such as lactose. In certain embodiments, the methods herein comprise administering an inhalable pharmaceutical composition comprising a suspension of particles of the ensifentrine in a diluent.

As described herein, the ensifentrine particles may be dispersed in a diluent (e.g., forming a suspension of particles in the diluent). A diluent may comprise water or a buffer (e.g., phosphate buffer). In some embodiments, a diluent comprises water. In some embodiments, a diluent comprises a buffer. In some embodiments, a diluent comprises a phosphate buffer.

In some embodiments, the ensifentrine particles may be dispersed in a sterile liquid vehicle. In some embodiments, the sterile liquid vehicle comprises one or more diluents. In some embodiments, the sterile liquid vehicle comprises a single diluent, such as water.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the invention described herein may be employed in practicing the disclosure. It is intended that the following claims define the scope of the disclosure and that methods and structures within the scope of these claims and their equivalents be covered thereby.

### EXAMPLES

### Example 1: Ensifentrine Treatment of Subjects with COPD

A clinical study was conducted to determine the efficacy of ensifentrine in treating COPD compared with placebo. Ensifentrine was administered by nebulizer at a dose of 3 mg twice daily (BID) for 24 weeks. The study was a multicenter, randomized, double-blind, parallel group, placebo-controlled trial with around 800 patients and 5:3 randomization.

The study population included patients aged 40-80 years with moderate to severe COPD (FEV₁ 30%-70% p.n., FEV₁/forced vital capacity (FVC) ratio<0.7, with mMRC≥2). The randomization stratified (a) the use of stable background maintenance LAMA or LABA therapy use (approx. 50% yes or no) and (b) cigarette smoking (current or former). Inhaled corticosteroid (ICS) maintenance therapy was permitted in up to 20% of patients under certain provisions.

The primary endpoint of the study was change from baseline in average FEV₁ area under the curve (AUC_{0-12 h}) over 4 h post-dose at week 12; morning trough FEV₁ at week 12; and other endpoints including moderate/severe COPD exacerbations frequency over 24 weeks.

### Methods

Baseline FEV₁ is the mean of the two measurements taken before study medication on the day of first dosing, i.e., ≤40 minutes and just prior to dosing, both pre-dose on day 1. Average FEV₁ AUC_{0-12 h} is defined as area under the curve over 12 h of the FEV₁, divided by 12 h.

Plasma concentrations of ensifentrine for the assessment of pharmacokinetic (PK) parameters were measured using validated bioanalytical methods for ensifentrine in human plasma with LLOQ of 5 μg/mL. Samples were taken on the following schedule:
Week 6: 1.0 h (±0.5 h) and 2.5 h (±0.5 h) (even sites) or 1.5 h and 4 h (±1 h) (odd sites);
Week 12: pre-dose (−0.5 h), 4 to 6 h, and 8 to 12 h (even sites) or 0.5 h (±0.25 h), 3 h (±0.5 h), and 6 to 8 h (odd sites);
Week 24: pre-dose (−0.5 h) and 1.5 h (even sites) or pre-dose (−0.5 h) and 1 h (±0.5 h) (odd sites)

### Formulation

The investigational product and placebo were provided in 2.5 mL unit dose format in an ampule and administered via a nebulizer. The formulation of the investigational product (ensifentrine suspension formulation comprising crystalline polymorph Form I of ensifentrine) and placebo are shown in Table 2.

TABLE 2

| Constituent | Concentration (mg/mL) |
|---|---|
| Ensifentrine | 1.2 (for the active) |
| particles (RPL554) | or 0 (for Placebo) |
| Dv10 = 0.5 μm | |
| Dv50 = 1.5 μm | |
| Dv90 = 3.6 μm | |
| Polysorbate 20 | 0.50 |
| (Tween 20) | |
| Sorbitan Monolaurate | 0.05 |
| (Span 20) | |
| Sodium Dihydrogen | 0.744 |
| Phosphate Dihydrate | |
| Disodium Hydrogen | 0.853 |
| Phosphate Dihydrate | |
| Sodium Chloride | 8.60 |
| Water | q.s. to 1 mL |

### Results—Chronic Bronchitis

In the study, 1,548 subjects were identified as either being diagnosed with chronic bronchitis or not being diagnosed with chronic bronchitis. Of the 1,548 subjects, 1,111 were identified as being diagnosed with chronic bronchitis, and 437 were identified as not being diagnosed with chronic bronchitis. The chronic bronchitis subgroup population was assessed for primary and secondary efficacy endpoints. For subjects with a chronic bronchitis diagnosis, the average FEV₁ AUC_{0-12 h} at week 12 of the study was 0.08 L (80 mL). For subjects without chronic bronchitis, the average FEV₁ AUC_{0-12 h} at week 12 of the study was 0.12 L (120 mL). The results of the FEV₁ AUC_{0-12 h} study are shown in Table 3.

TABLE 3

| Chronic | Ensifentrine | Placebo | FEV₁ AUC_{0-12 h} (95% |
|---|---|---|---|
| Bronchitis | Events (%) | Events (%) | Confidence Interval) |
| Yes | 707 (72.5%) | 404 (70.4%) | 0.08 L (0.05, 0.10) |
| No | 268 (27.5%) | 169 (29.4%) | 0.12 L (0.08, 0.16) |

The subjects in the chronic bronchitis subgroups were assessed for changes from baseline FEV₁ to peak FEV₁ at week 12 of the study. Subjects with chronic bronchitis demonstrated a change from baseline FEV₁ to peak FEV₁ of 0.14 L. Subjects without chronic bronchitis demonstrated a change from baseline FEV₁ to peak FEV₁ of 0.17 L. The results of the change from baseline FEV₁ to peak FEV₁ study are shown in Table 4.

TABLE 4

| Chronic | Ensifentrine | Placebo | Change from baseline |
|---|---|---|---|
| Bronchitis | Events (%) | Events (%) | to peak FEV₁ (95% CI) |
| Yes | 707 (72.5%) | 404 (70.4%) | 0.14 L (0.11, 0.17) |
| No | 268 (27.5%) | 169 (29.4%) | 0.17 L (0.13, 0.21) |

The subjects in the chronic bronchitis subgroups were assessed for FEV₁ AUC_{0-4 h} at week 12 of the study. Subjects with chronic bronchitis demonstrated an average FEV₁ AUC_{0-4 h} of 0.13 L. Subjects without chronic bronchitis demonstrated an average FEV₁ AUC_{0-4 h} of 0.16 L. The results of FEV₁ AUC_{0-4 h} study are shown in Table 5.

TABLE 5

| Chronic | Ensifentrine | Placebo | FEV₁ AUC_{0-4 h} |
|---|---|---|---|
| Bronchitis | Events (%) | Events (%) | (95% CI) |
| Yes | 707 (72.5%) | 404 (70.4%) | 0.13 L (0.10, 0.16) |
| No | 268 (27.5%) | 169 (29.4%) | 0.16 L (0.11, 0.20) |

### Results—Trough Lung Function

Trough lung function was assessed in subjects with varying severity levels of COPD (e.g., mild or moderate or severe or very severe). Trough lung function was assessed by studying the change from baseline FEV₁ to morning trough FEV₁ in 1,548 subjects. Of the 1,548 subjects in the study, 868 were identifying as having mild or moderate COPD, whereas 680 subjects were identified as having severe or very severe COPD. Subjects with mild or moderate COPD exhibited an average change from baseline FEV₁ to morning trough FEV₁ of 0.06 L. Subjects with severe or very severe COPD exhibited an average change from baseline FEV₁ to morning trough FEV₁ of 0.02 L. The results of the change in baseline FEV₁ to morning trough FEV₁ are shown in Table 6.

TABLE 6

| COPD | Ensifentrine | Placebo | Change from Baseline to |
|---|---|---|---|
| Severity | Events (%) | Events (%) | Morning Trough FEV₁ (95% CI) |
| Mild or moderate | 561 (57.5%) | 307 (53.5%) | 0.06 L (0.03, 0.09) |
| Severe or | 414 (42.5%) | 266 (46.3%) | 0.02 L (0.01, 0.05) |
| very severe | | | |

### Results—COPD Reversibility Status

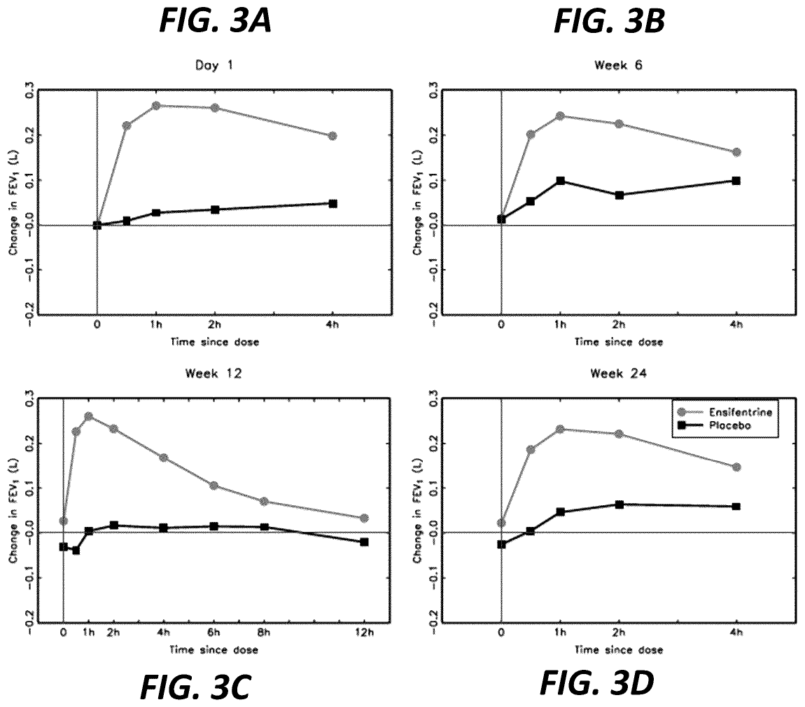

Ensifentrine's ability to treat COPD in individual identified with a reversibility state of COPD being reversible or non-reversible was assessed during the study. Among the participants in the study, 437 were identified to have COPD with a reversible reversibility status, whereas 1,111 were identified to have COPD with a non-reversible reversibility status. The ability of ensifentrine to treat COPD in these subgroup populations was assessed via baseline FEV₁ to peak FEV₁ and FEV₁ AUC_{0-4 h}. Subjects with a reversible status of COPD demonstrated an average change from baseline FEV₁ to peak FEV₁ of 0.17 L. Subjects with a non-reversible status of COPD demonstrated an average change from baseline FEV₁ to peak FEV₁ of 0.13 L. The results of the change from baseline FEV₁ to peak FEV₁ study are shown in Table 7. Time profiles of FEV₁ change from baseline for subjects with a reversible status of COPD are shown in FIG. 3A-D.

TABLE 7

| Reversibility | Ensifentrine | Placebo | Change from Baseline |
|---|---|---|---|
| Status | Events (%) | Events (%) | to Peak FEV₁ (95% CI) |
| Reversible | 289 (29.6%) | 148 (25.8%) | 0.17 L (0.12, 0.22) |
| Non-reversible | 686 (70.4%) | 425 (74.0%) | 0.13 L (0.10, 0.16) |

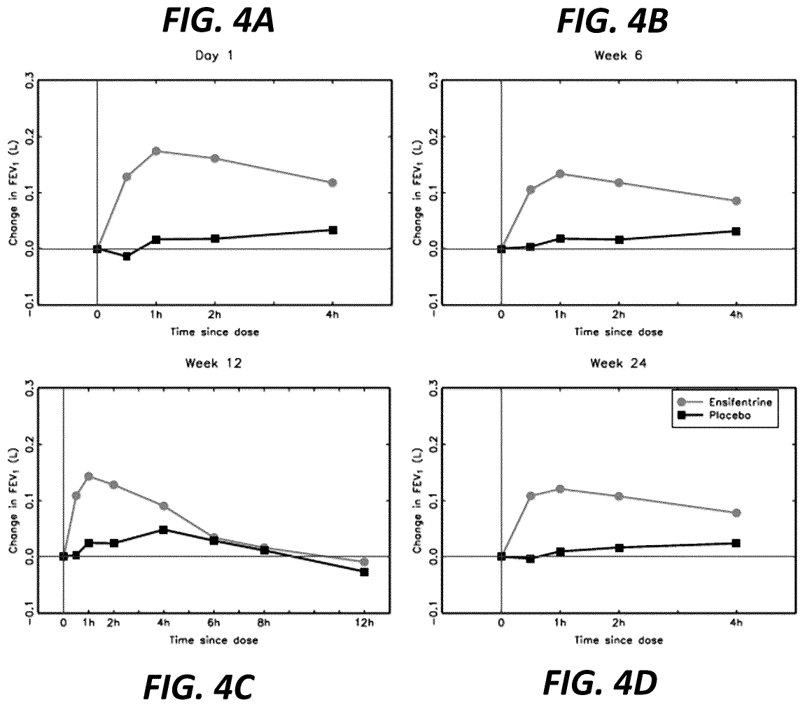

Subjects with a reversible status of COPD demonstrated an average FEV₁ AUC_{0-4 h} of 0.16 L. Subjects with a non-reversible status of COPD demonstrated an average FEV₁ AUC_{0-4 h} of 0.12 L. These results are shown in Table 8. Time profiles of FEV₁ change from baseline for subjects with a non-reversible status of COPD are shown in FIG. 4A-D.

TABLE 8

| Reversibility | Ensifentrine | Placebo | FEV₁ AUC_{0-4 h} |
|---|---|---|---|
| Status | Events (%) | Events (%) | (95% CI) |
| Reversible | 289 (29.6%) | 148 (25.8%) | 0.16 L (0.12, 0.21) |
| Non-reversible | 686 (70.4%) | 425 (74.0%) | 0.12 L (0.10, 0.15) |

### Results—COPD Exacerbations

Ensifentrine's ability to reduce the frequency and or severity of COPD exacerbations was studied in 1,549 subjects. Ensifentrine's ability to reduce the frequency and or severity of COPD exacerbations was assessed in various subgroup populations. In one part of the study, subjects were assessed based on whether they were diagnosed with mild or moderate COPD or severe or very severe COPD. Subjects with mild or moderate COPD exhibited an exacerbation rate ratio after 24 weeks of administration of 0.34. Subjects with severe or very severe COPD exhibited an exacerbation rate ratio after 24 weeks of administration of 0.93. An exacerbation rate ratio below one is indicative that the ensifentrine performs better at reducing exacerbations than the placebo. Lower exacerbation rate ratios may be correlated with increased efficacy of ensifentrine in decreasing the frequency/severity of COPD exacerbations. The results of this study are summarized in Table 9.

TABLE 9

| COPD | Ensifentrine | Placebo | Exacerbation Rate |
|---|---|---|---|
| Severity | Events (%) | Events (%) | Ratio (95% CI) |
| Mild or moderate | 561 (57.5%) | 307 (53.5%) | 0.34 (0.21, 0.55) |
| Severe of | 414 (42.5%) | 267 (46.5%) | 0.93 (0.63, 1.37) |
| very severe | | | |

These subjects were also assessed for the time to first moderate/severe COPD exacerbation, as measured by a hazard ratio from a Cox model. Subjects with mild or moderate COPD demonstrated an average hazard ratio of 0.35. Subjects with severe or very severe COPD demonstrated an average hazard ratio of 0.90. A hazard ratio of below 1.0 is indicative that the ensifentrine performs better than the placebo at reducing the severity/frequency of COPD exacerbations. A lower hazard ratio (e.g., closer to 0) is further indicative that ensifentrine performs better than a placebo. These results are summarized in Table 10.

TABLE 10

| | | | Time to First Moderate/ |
|---|---|---|---|
| | | | Severe Exacerbation |
| COPD | Ensifentrine | Placebo | (Hazard Ratio) |
| Severity | Events (%) | Events (%) | (95% CI) |
| Mild or moderate | 561 (57.5%) | 307 (53.5%) | 0.35 (0.21, 0.57) |
| Severe of | 414 (42.5%) | 267 (46.5%) | 0.90 (0.60, 1.35) |
| very severe | | | |

In another part of this study, subjects were assessed based on blood eosinophil concentration (e.g., greater than 150 cells/μL or less than or equal to 150 cells/μL). Reductions in exacerbations were observed in subjects with baseline blood eosinophil concentrations of greater than 150 cells/μL and with concentrations of less than or equal to 150 cells/μL. This may indicate effects on both Type 1 inflammation in COPD patients (more neutrophilic) and Type 2 inflammation (more eosinophilic). This may be in contrast to data with the anti-inflammatory effects of ICS, which are known to be limited to COPD patients with high blood eosinophil levels.

### Results—ICS Use

Ensifentrine was studied for its ability to treat COPD in subjects using inhalable corticosteroids (ICS) while also using a LABA. Changes in TDI were demonstrated between subjects being administered the ensifentrine pharmaceutical composition as compared to subjects being administered a placebo. The results of this study are summarized in Table 11. The results indicate that subjects being administered ensifentrine while taking LABA and ICS exhibited improvements in dyspnea as compared to subjects administered a placebo.

TABLE 11

| Transition Dyspnea Index | Statistic | Ensifentrine | Placebo |
|---|---|---|---|
| COPD Medication | LS Mean | 1.394 (0.4748) | 0.626 (0.4958) |

| | | |
|---|---|---|
| LABA + ICS | Estimate | 0.768 (0.047, |
| | (Ensi-Pla) | 1.489) |
| | P-Value | 0.0367 |

### EMBODIMENTS

Additional embodiments include:

1. A method of treating chronic obstructive pulmonary disease (COPD) in a human subject not having chronic bronchitis, the method comprising administering to the human subject a liquid pharmaceutical composition comprising a therapeutically effective amount of ensifentrine or a pharmaceutically acceptable salt thereof.
2. The method of embodiment 1, wherein the human subject has a mean change in baseline to peak FEV₁ (forced expiratory volume in 1 second) of from about 0.1 L to about 0.2 L.
3. The method of any one of the preceding embodiments, wherein the human subject has a mean change in baseline to peak FEV₁ of about 0.17 L.
4. The method of embodiment 2 or 3, wherein the mean change in baseline to peak FEV₁ of the human subject not having chronic bronchitis is at least 15% greater than the mean change in baseline to peak FEV₁ of a human subject who does have chronic bronchitis being administered the liquid pharmaceutical composition.
5. The method of embodiment 4, wherein the mean change in baseline to peak FEV₁ of the human subject not having chronic bronchitis is at least 20% greater than the mean change in baseline to peak FEV₁ of a human subject who does have chronic bronchitis.
6. The method of any one of the preceding embodiments, wherein the human subject has a mean FEV₁ AUC_{0-4 h} of from about 0.1 L to about 0.2 L.
7. The method of any one of the preceding embodiments, wherein the human subject has a mean FEV₁ AUC_{0-4 h} of about 0.16 L.
8. The method of embodiment 6 or 7, wherein the mean FEV₁ AUC_{0-4 h} of the human subject not having chronic bronchitis is at least 20% greater than the mean FEV₁ AUC_{0-4 h} of a human subject who does have chronic bronchitis being administered the liquid pharmaceutical composition.
9. The method of any one of the preceding embodiments, wherein the human subject has a mean FEV₁ AUC_{0-12 h} of from about 0.1 L to about 0.2 L.
10. The method of embodiment 9, wherein the human subject has a mean FEV₁ AUC_{0-12 h} of about 0.12 L.
11. The method of embodiment 9 or 10, wherein the mean FEV₁ AUC_{0-12 h} of the human subject not having chronic bronchitis is at least 30% greater than the mean FEV₁ AUC_{0-12 h} of a human subject who does have chronic bronchitis being administered the liquid pharmaceutical composition.
12. The method of embodiment 11, wherein the mean FEV₁ AUC_{0-12 h} of the human subject not having chronic bronchitis is at least 40% greater than the mean FEV₁ AUC_{0-12 h} of a human subject who does have chronic bronchitis being administered the liquid pharmaceutical composition.
13. The method of any one of embodiments 2-12, wherein the mean change in baseline to peak FEV₁ or mean FEV₁ AUC_{0-12 h} is measured after 12 weeks of administration of the liquid pharmaceutical composition.
14. A method of increasing trough lung function in a human subject suffering from moderate chronic obstructive pulmonary disease (COPD), the method comprising administering to the human subject a liquid pharmaceutical composition comprising a therapeutically effective amount of ensifentrine or a pharmaceutically acceptable salt thereof.
15. The method of embodiment 14, wherein the human subject has a change in baseline to morning trough FEV₁ of from about 30 mL to about 90 mL.
16. The method of embodiment 14 or 15, wherein the human subject has a change in baseline to morning trough FEV₁ of about 60 mL.
17. The method of embodiment 15 or 16, wherein the change in baseline to morning trough FEV₁ of the human subject suffering from moderate COPD is at least 100% greater than a change in baseline to morning trough FEV₁ of a human subject suffering from severe or very severe COPD being administered the liquid pharmaceutical composition.
18. The method of any one of embodiments 15-17, wherein the change in baseline to morning trough FEV₁ is measured after 12 weeks of administration of the liquid pharmaceutical composition.
19. A method of treating chronic obstructive pulmonary disease (COPD) having a reversibility status of reversible in a human subject, the method comprising administering to the human subject a liquid pharmaceutical composition comprising a therapeutically effective amount of ensifentrine or a pharmaceutically acceptable salt thereof.
20. The method of embodiment 19, wherein the human subject having a reversible status of COPD has a change from baseline FEV₁ to peak FEV₁ of from about 0.1 L to about 0.2 L.
21. The method of embodiment 20, wherein the human subject having a reversible status of COPD has a change from baseline FEV₁ to peak FEV₁ of about 0.17 L.
22. The method of embodiment 20 or 21, wherein the change from baseline FEV₁ to peak FEV₁ for a human subject having a reversible status of COPD is at least 20% greater than a change from baseline FEV₁ to peak FEV₁ for a human subject having a non-reversible status of COPD being administered the liquid pharmaceutical composition.
23. The method of embodiment 22, wherein the change from baseline FEV₁ to peak FEV₁ for a human subject having a reversible status of COPD is about 25% greater than a change from baseline FEV₁ to peak FEV₁ for a human subject having a non-reversible status of COPD being administered the liquid pharmaceutical composition.
24. The method of any one of embodiments 19-23, wherein the human subject having a reversible status of COPD has a FEV₁ AUC_{0-4 h} of from about 0.1 L to about 0.2 L.
25. The method of embodiment 24, wherein the human subject having a reversible status of COPD has a FEV₁ AUC_{0-4 h} of about 0.16 L.
26. The method of embodiment 24 or 25, wherein the FEV₁ AUC_{0-4 h} for a human subject having a reversible status of COPD is at least 25% greater than a FEV₁ AUC_{0-4 h} for a human subject having a non-reversible status of COPD being administered the liquid pharmaceutical composition.
27. The method of embodiment 26, wherein the FEV₁ AUC_{0-4 h} for a human subject having a reversible status of COPD is about 33% greater than a FEV₁ AUC_{0-4 h} for a human subject having a non-reversible status of COPD being administered the liquid pharmaceutical composition.
28. A method of decreasing the frequency and/or severity of chronic obstructive pulmonary disease (COPD) exacerbations in a human subject suffering from moderate COPD, the method comprising administering to the human subject a liquid pharmaceutical composition comprising a therapeutically effective amount of ensifentrine or a pharmaceutically acceptable salt thereof.
29. The method of embodiment 28, wherein the COPD exacerbations are moderate to severe COPD exacerbations.
30. The method of embodiment 29, wherein the moderate to severe COPD exacerbations are reduced by at least 30%.
31. The method of embodiment 30, wherein the moderate to severe COPD exacerbations are reduced by about 36%.
32. The method of embodiment 30, wherein the moderate to severe COPD exacerbations are reduced by about 44%.
33. The method of any one of embodiments 28-32, wherein decreasing the frequency and/or severity of COPD exacerbations comprises increasing the amount of time to a first COPD exacerbation, as measured by a hazard ratio.
34. The method of embodiment 33, wherein the hazard ratio for the human subject having moderate COPD is at least 40% lower than a hazard ratio for a subject having severe or very severe COPD being administered the liquid pharmaceutical composition.
35. The method of embodiment 34, wherein the hazard ratio for the human subject having moderate COPD is about 60% lower than a hazard ratio for a subject having severe or very severe COPD being administered the liquid pharmaceutical composition.
36. The method of any one of embodiments 28-35, wherein decreasing the frequency and/or severity of COPD exacerbations comprises decreasing exacerbation risk (e.g., as measured by time to first event).
37. The method of embodiment 36, wherein the exacerbation risk is reduced by at least 40%.
38. The method of embodiment 37, wherein the exacerbation risk is reduced by at least 50%.
39. The method of any one of embodiments 28-38, wherein the human subject comprises a baseline blood eosinophils concentration of less than or equal to 150 cells/μL.
40. The method of any one of embodiments 28-39, wherein an exacerbation rate ratio for the human subject having moderate COPD is at least 50% lower than an exacerbation rate for a human subject having severe or very severe COPD being administered the liquid pharmaceutical composition.
41. The method of embodiment 40, wherein the exacerbation rate ratio for the human subject having moderate COPD is about 60% lower than an exacerbation rate for a human subject having severe or very severe COPD being administered the liquid pharmaceutical composition.
42. A method of treating chronic obstructive pulmonary disease (COPD) in a human subject being administered inhalable corticosteroids (ICS) and a long acting beta agonist (LABA), the method comprising administering to the human subject a liquid pharmaceutical composition comprising a therapeutically effective amount of ensifentrine or a pharmaceutically acceptable salt thereof.
43. The method of embodiment 42, wherein a change in TDI (transition dyspnea index) is at least 100% greater for the human subject being administered the liquid pharmaceutical composition than for a human subject being administered a placebo.
44. The method of embodiment 43, wherein a change in TDI (transition dyspnea index) is about 125% greater for the human subject being administered the liquid pharmaceutical composition than for a human subject being administered a placebo.
45. The method of any one of the preceding embodiments, wherein the administering is by inhalation.
46. The method of any one of the preceding embodiments, wherein the inhalation is by nebulizer.
47. The method of any one of the preceding embodiments, wherein the ensifentrine is ensifentrine free base.
48. The method of any one of the preceding embodiments, wherein the administering to the human subject is at least once, twice, or three times per day.
49. The method of any one of the preceding embodiments, wherein the administering to the human subject is at least twice a day.
50. The method of any one of the preceding embodiments, wherein the therapeutically effective amount of ensifentrine or the pharmaceutically acceptable salt thereof is of from about 2 mg to about 4 mg.
51. The method of any one of the preceding embodiments, wherein the therapeutically effective amount of ensifentrine or the pharmaceutically acceptable salt thereof is 3 mg.
52. The method of any one of the preceding embodiments, wherein the therapeutically effective amount of ensifentrine or the pharmaceutically acceptable salt thereof comprises a total daily dose of the ensifentrine or the pharmaceutically acceptable salt thereof of from about 0.5 mg to about 10 mg.
53. The method of any one of the preceding embodiments, wherein the therapeutically effective amount of ensifentrine or the pharmaceutically acceptable salt thereof comprises a total daily dose of the ensifentrine or the pharmaceutically acceptable salt thereof of from about 5 mg to about 7 mg.
54. The method of any one of the preceding embodiments, wherein the administering to the human subject comprises a first dose and a second dose.
55. The method of embodiment 54, wherein the first dose comprises of from about 2 mg to about 4 mg of ensifentrine or the pharmaceutically acceptable salt thereof and the second dose comprises of from about 2 mg to about 4 mg of ensifentrine or the pharmaceutically acceptable salt thereof.
56. The method of embodiment 54, wherein the first and the second dose comprise about 3 mg of the ensifentrine or the pharmaceutically acceptable salt thereof.
57. The method of any one of embodiments 54-56, wherein the first dose is administered in the morning and the second dose is administered in the evening.
58. The method of any one of embodiments 54-56, wherein the first dose is administered within three hours after the human subject waking, and the second dose is administered within three hours before the human subject sleeps.
59. The method of any one of embodiments 54-56, wherein the first dose and the second dose are administered to the human subject about 10 h to about 14 h apart.
60. The method of any one of the preceding embodiments, wherein the method comprises administering the liquid pharmaceutical composition to the subject at least once per day for at least 8 weeks.
61. The method of any one of the preceding embodiments, wherein the administering to the human subject is at least once per day for at least 16 weeks.
62. The method of any one of the preceding embodiments, wherein the administering to the human subject is at least one per day for at least 24 weeks.
63. The method of any one of the preceding embodiments, wherein the liquid pharmaceutical composition is an inhalable pharmaceutical composition.
64. The method of any one of the preceding embodiments, wherein the liquid pharmaceutical composition comprises a suspension of ensifentrine particles comprising the ensifentrine or a pharmaceutically acceptable salt thereof in a diluent.
65. The method of embodiment 64, wherein the ensifentrine particles comprise at least 95% by weight of the ensifentrine or the pharmaceutically acceptable salt thereof.
66. The method of embodiment 64, wherein the ensifentrine particles comprise at least 95% by weight of ensifentrine free base.
67. The method of any one of embodiments 64-66, wherein the ensifentrine is in the form of a crystalline solid consisting of greater than 99% by weight of ensifentrine free base, at least 95% in the polymorphic form of a thermodynamically stable polymorph (I) of ensifentrine free base having the following structural parameters obtained by single crystal analysis:

| Wavelength | 0.71073 Å | |
|---|---|---|
| Crystal system | Triclinic | |
| Space group | P-1 | |
| Unit cell dimensions | a = 8.1246(4) Å | α = 91.583(4)° |
| | b = 11.4573(5) Å | β = 90.299(4)° |
| | c = 13.2398(6) Å | γ = 99.628(4)° |
| Volume | 1214.56(10) Å³ | |
| Z | 2 | |

68. The method of any one of embodiments 64-67, wherein the ensifentrine particles comprise:
of from about 0% to about 0.6% by weight of 1,3-bis(2-(2-(mesitylimino)-9,10-dimethoxy-4-oxo-6,7-dihydro-2H-pyrimido[6,1-a]isoquinolin-3(4H)-yl)ethyl)urea (BMIQU) relative to the total weight of the ensifentrine or the pharmaceutically acceptable salt thereof.
   69. The method of any one of embodiments 64-68, wherein the ensifentrine particles comprise of from about 0% to about 0.5% by weight of a biuret impurity of formula (A) relative to the total weight of the ensifentrine or the pharmaceutically acceptable salt thereof:

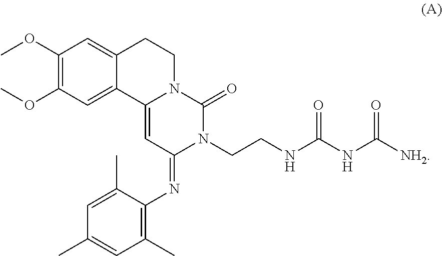 [SMILES: COc1cc2c(cc1OC)-c1c/c(=N\c3c(C)cc(C)cc3C)n(CCNC(=O)NC(N)=O)c(=O)n1CC2]

70. The method of embodiment 69, wherein the ensifentrine particles comprise of from about 0% to about 0.3% by weight of the biuret impurity relative to the total weight of the ensifentrine or the pharmaceutically acceptable salt thereof.
71. The method of any one of embodiments 68-70, wherein the ensifentrine particles comprise of from 0.01% to 0.3% by weight of BMIQU relative to the total weight of ensifentrine or a pharmaceutically acceptable salt thereof.
72. The method of any one of embodiments 64-71, wherein the liquid pharmaceutical composition comprises:
a. ensifentrine particles;
b. a buffer;
c. one or more surfactants; and
d. a tonicity adjuster.
   73. The method of any one of embodiments 64-72, wherein the liquid pharmaceutical composition comprises:
a. ensifentrine particles at a concentration of from about 1 mg/mL to about 1.4 mg/mL;
b. a buffer at a concentration of from about 1 mg/mL to about 2.0 mg/mL;
c. one or more surfactants at a total concentration of from about 0.3 mg/mL to about 0.8 mg/mL; and
d. a tonicity adjuster at a concentration of from about 5 mg/mL to about 10 mg/mL.
   74. The method of any one of embodiments 64-73, wherein the liquid pharmaceutical composition comprises:
a. ensifentrine particles at a concentration of from about 1 mg/mL to about 1.4 mg/mL;
b. polysorbate 20 (Tween 20) at a concentration of from about 0.3 mg/mL to about 0.7 mg/mL;
c. sorbitan monolaurate (Span 20) at a concentration of from 0 mg/mL to about 0.1 mg/mL;
d. sodium dihydrogen phosphate dihydrate at a concentration of from about 0.5 mg/mL to about 1 mg/mL;
e. disodium hydrogen phosphate dihydrate at a concentration of from about 0.5 mg/mL to about 1 mg/mL; and
f. sodium chloride at a concentration of from about 5 mg/mL to about 10 mg/mL.
   75. The method of any one of embodiments 64-74, wherein the liquid pharmaceutical composition comprises:
a. 1.2 mg/mL ensifentrine particles;
b. 0.5 mg/mL polysorbate 20;
c. 0.05 mg/mL sorbitan monolaurate;
d. 0.744 mg/mL sodium dihydrogen phosphate dihydrate;
e. 0.853 mg/mL disodium hydrogen phosphate dihydrate;
f. 8.6 mg/mL sodium chloride; and
g. water.
   76. The method of any one of embodiments 64-74, wherein the liquid pharmaceutical composition is a suspension comprising:
a. 1.4 mg/mL ensifentrine particles;
b. 0.55 mg/ml polysorbate 20;
c. 0.744 mg/ml sodium dihydrogen phosphate dihydrate;
d. 0.853 mg/ml disodium hydrogen phosphate dihydrate;
e. 8.6 mg/ml sodium chloride; and
f. water.
   77. The method of any one of embodiments 64-74, wherein the liquid pharmaceutical composition is a suspension comprising:
a. 1.3 mg/mL ensifentrine particles;
b. 0.55 mg/ml sorbitan monolaurate;
c. 0.744 mg/ml sodium dihydrogen phosphate dihydrate;
d. 0.853 mg/ml disodium hydrogen phosphate dihydrate;
e. 8.6 mg/ml sodium chloride; and
f. water.
   78. The method of any one of embodiments 64-74, wherein the liquid pharmaceutical composition is a suspension comprising:
a. 1.3 mg/mL ensifentrine particles;
b. 0.5 mg/ml polysorbate 20;
c. 0.05 mg/ml sorbitan monolaurate;
d. 1.1 mg/ml sodium dihydrogen phosphate dihydrate;
e. 0.9 mg/ml disodium hydrogen phosphate dihydrate;
f. 13 mg/ml sodium chloride; and
g. water.
   79. The method of any one of embodiments 64-74, wherein the liquid pharmaceutical composition is a suspension comprising:
a. 1.2 mg/mL ensifentrine particles;
b. 0.4 mg/ml polysorbate 20;
c. 0.10 mg/ml sorbitan monolaurate;
d. 0.744 mg/ml sodium dihydrogen phosphate dihydrate;
e. 0.853 mg/ml disodium hydrogen phosphate dihydrate;
f. 8.6 mg/ml sodium chloride; and
g. water.
   80. The method of any one of embodiments 64-74, wherein the liquid pharmaceutical composition is a suspension comprising:
a. 1.4 mg/mL ensifentrine particles;
b. 0.4 mg/ml polysorbate 20;
c. 0.04 mg/ml sorbitan monolaurate;
d. 1.2 mg/ml sodium dihydrogen phosphate dihydrate;
e. 8.6 mg/ml sodium chloride; and
f. water.
   81. The method of any one of embodiments 64-74, wherein the liquid pharmaceutical composition comprises:
a. the ensifentrine particles at a concentration of from 1.0 to 2.0 mg/mL;
b. one or more surfactants at a total concentration of from 0.3 to 0.7 mg/mL;
c. one or more buffers at a total concentration of from 1.0 to 2.0 mg/ml;
d. one or more tonicity adjusters at a concentration of from 1.0 to 15.0 mg/ml and
e. water.
   82. The method of any one of embodiments 72-81, wherein a total weight of the ensifentrine or the pharmaceutically acceptable salt thereof in the liquid pharmaceutical composition is of from about 2.7 mg to about 3.3 mg.
   83. The method of any one of the preceding embodiments, wherein the ensifentrine or the pharmaceutically acceptable salt thereof is used in combination with a muscarinic receptor antagonist, a beta-adrenergic receptor agonist, or an inhaled corticosteroid.
   84. Use of the method of any one of embodiments 1-83, for treating chronic obstructive pulmonary disease (COPD) in a human subject.

Although the foregoing refers to particular preferred embodiments, it will be understood that the present invention is not so limited. It will occur to those of ordinary skill in the art that various modifications may be made to the disclosed embodiments and that such modifications are intended to be within the scope of the present invention.

All of the publications, patent applications and patents cited in this specification are incorporated herein by reference in their entirety.

We claim:

1. A method of treating chronic obstructive pulmonary disease (COPD) in a human subject not having chronic bronchitis, the method comprising administering to the human subject a liquid pharmaceutical composition comprising a therapeutically effective amount of ensifentrine or a pharmaceutically acceptable salt thereof, wherein the administering to the human subject is at least once, twice, or three times per day.

2. The method of claim 1, wherein the human subject has a mean change in baseline to peak FEV₁ (forced expiratory volume in 1 second) of from about 0.1 L to about 0.2 L.

3. The method of claim 2, wherein the mean change in baseline to peak FEV₁ of the human subject not having chronic bronchitis is at least 15% greater than the mean change in baseline to peak FEV₁ of a human subject who does have chronic bronchitis being administered the liquid pharmaceutical composition.

4. The method of claim 1, wherein the human subject has a mean FEV₁ AUC_{0-4 h} of from about 0.1 L to about 0.2 L.

5. The method of claim 4, wherein the mean FEV₁ AUC_{0-4 h} of the human subject not having chronic bronchitis is at least 20% greater than the mean FEV₁ AUC_{0-4 h} of a human subject who does have chronic bronchitis being administered the liquid pharmaceutical composition.

6. The method of claim 1, wherein the human subject has a mean FEV₁ AUC_{0-12 h} of from about 0.1 L to about 0.2 L.

7. The method of claim 6, wherein the mean FEV₁ AUC_{0-12 h} of the human subject not having chronic bronchitis is at least 30% greater than the mean FEV₁ AUC_{0-12 h} of a human subject who does have chronic bronchitis being administered the liquid pharmaceutical composition.

8. The method of claim 1, wherein the mean change in baseline to peak FEV₁ or mean FEV₁ AUC_{0-12 h} is measured after 12 weeks of administration of the liquid pharmaceutical composition.

9. The method of claim 1, wherein the therapeutically effective amount of ensifentrine or the pharmaceutically acceptable salt thereof is of from about 2 mg to about 4 mg.

10. The method of claim 1, wherein the therapeutically effective amount of ensifentrine or the pharmaceutically acceptable salt thereof comprises a total daily dose of the ensifentrine or the pharmaceutically acceptable salt thereof of from about 0.5 mg to about 10 mg.

11. The method of claim 1, wherein a first dose comprises of from about 2 mg to about 4 mg of ensifentrine or the pharmaceutically acceptable salt thereof and a second dose comprises of from about 2 mg to about 4 mg of ensifentrine or the pharmaceutically acceptable salt thereof.

12. The method of claim 1, wherein the liquid pharmaceutical composition is an inhalable pharmaceutical composition.

13. The method of claim 1, wherein the liquid pharmaceutical composition comprises a suspension of ensifentrine particles comprising the ensifentrine or a pharmaceutically acceptable salt thereof in a diluent, wherein the ensifentrine particles comprise at least 95% by weight of the ensifentrine or the pharmaceutically acceptable salt thereof.

14. The method of claim 13, wherein the liquid pharmaceutical composition comprises:
a. ensifentrine particles;
b. a buffer;
c. one or more surfactants; and
d. a tonicity adjuster,
wherein a total weight of the ensifentrine or the pharmaceutically acceptable salt thereof in the liquid pharmaceutical composition is of from about 2.7 mg to about 3.3 mg.

15. The method of claim 14, wherein the liquid pharmaceutical composition comprises:
a. ensifentrine particles at a concentration of from about 1 mg/mL to about 1.4 mg/mL;
b. a buffer at a concentration of from about 1 mg/mL to about 2.0 mg/mL;
c. one or more surfactants at a total concentration of from about 0.3 mg/mL to about 0.8 mg/mL; and
d. a tonicity adjuster at a concentration of from about 5 mg/mL to about 10 mg/mL.

16. The method of claim 14, wherein the liquid pharmaceutical composition comprises:
a. ensifentrine particles at a concentration of from about 1 mg/mL to about 1.4 mg/mL;
b. polysorbate 20 at a concentration of from about 0.3 mg/mL to about 0.7 mg/mL;
c. sorbitan monolaurate at a concentration of from 0 mg/mL to about 0.1 mg/mL;
d. sodium dihydrogen phosphate dihydrate at a concentration of from about 0.5 mg/mL to about 1 mg/mL;
e. disodium hydrogen phosphate dihydrate at a concentration of from about 0.5 mg/mL to about 1 mg/mL; and
f. sodium chloride at a concentration of from about 5 mg/mL to about 10 mg/mL.

17. The method of claim 14, wherein the liquid pharmaceutical composition comprises:
a. 1.2 mg/mL ensifentrine particles;
b. 0.5 mg/mL polysorbate 20;
c. 0.05 mg/mL sorbitan monolaurate;
d. 0.744 mg/mL sodium dihydrogen phosphate dihydrate;
e. 0.853 mg/mL disodium hydrogen phosphate dihydrate;
f. 8.6 mg/mL sodium chloride; and
g. water.

18. The method of claim 14, wherein the liquid pharmaceutical composition is a suspension comprising:
a. 1.4 mg/mL ensifentrine particles;
b. 0.55 mg/ml polysorbate 20;
c. 0.744 mg/ml sodium dihydrogen phosphate dihydrate;
d. 0.853 mg/ml disodium hydrogen phosphate dihydrate;
e. 8.6 mg/ml sodium chloride; and
f. water.

19. The method of claim 14, wherein the liquid pharmaceutical composition is a suspension comprising:
a. 1.3 mg/mL ensifentrine particles;
b. 0.55 mg/ml sorbitan monolaurate;
c. 0.744 mg/ml sodium dihydrogen phosphate dihydrate;
d. 0.853 mg/ml disodium hydrogen phosphate dihydrate;
e. 8.6 mg/ml sodium chloride; and
f. water.

20. The method of claim 14, wherein the liquid pharmaceutical composition is a suspension comprising:
a. 1.3 mg/mL ensifentrine particles;
b. 0.5 mg/ml polysorbate 20;
c. 0.05 mg/ml sorbitan monolaurate;
d. 1.1 mg/ml sodium dihydrogen phosphate dihydrate;
e. 0.9 mg/ml disodium hydrogen phosphate dihydrate;
f. 13 mg/ml sodium chloride; and
g. water.

21. The method of claim 14, wherein the liquid pharmaceutical composition is a suspension comprising:
a. 1.2 mg/mL ensifentrine particles;
b. 0.4 mg/ml polysorbate 20;
c. 0.10 mg/ml sorbitan monolaurate;
d. 0.744 mg/ml sodium dihydrogen phosphate dihydrate;
e. 0.853 mg/ml disodium hydrogen phosphate dihydrate;
f. 8.6 mg/ml sodium chloride; and
g. water.

22. The method of claim 14, wherein the liquid pharmaceutical composition is a suspension comprising:
a. 1.4 mg/mL ensifentrine particles;
b. 0.4 mg/ml polysorbate 20;
c. 0.04 mg/ml sorbitan monolaurate;
d. 1.2 mg/ml sodium dihydrogen phosphate dihydrate;
e. 8.6 mg/ml sodium chloride; and
f. water.

23. The method of claim 14, wherein the liquid pharmaceutical composition comprises:
a. the ensifentrine particles at a concentration of from 1.0 to 2.0 mg/mL;
b. one or more surfactants at a total concentration of from 0.3 to 0.7 mg/mL;
c. one or more buffers at a total concentration of from 1.0 to 2.0 mg/ml;
d. one or more tonicity adjusters at a concentration of from 1.0 to 15.0 mg/ml and
e. water.

24. A method of treating chronic obstructive pulmonary disease (COPD) having a reversibility status of reversible in a human subject, the method comprising administering to the human subject a liquid pharmaceutical composition comprising a therapeutically effective amount of ensifentrine or a pharmaceutically acceptable salt thereof.

25. The method of claim 20, wherein the human subject having a reversible status of COPD has a change from baseline FEV₁ to peak FEV₁ of from about 0.1 L to about 0.2 L.

26. The method of claim 21, wherein the change from baseline FEV₁ to peak FEV₁ for a human subject having a reversible status of COPD is at least 20% greater than a change from baseline FEV₁ to peak FEV₁ for a human subject having a non-reversible status of COPD being administered the liquid pharmaceutical composition.

27. The method of claim 20, wherein the human subject having a reversible status of COPD has a FEV₁ AUC_{0-4 h} of from about 0.1 L to about 0.2 L.

28. The method of claim 23, wherein the FEV₁ AUC_{0-4 h} for a human subject having a reversible status of COPD is at least 25% greater than a FEV₁ AUC_{0-4 h} for a human subject having a non-reversible status of COPD being administered the liquid pharmaceutical composition.

29. A method of treating chronic obstructive pulmonary disease (COPD) in a human subject being administered inhalable corticosteroids (ICS) and a long acting beta agonist (LABA), the method comprising administering to the human subject a liquid pharmaceutical composition comprising a therapeutically effective amount of ensifentrine or a pharmaceutically acceptable salt thereof, wherein a change in TDI (transition dyspnea index) is at least 100% greater for the human subject being administered the liquid pharmaceutical composition than for a human subject being administered a placebo.

30. The method of claim 29, wherein a change in TDI (transition dyspnea index) is about 125% greater for the human subject being administered the liquid pharmaceutical composition than for a human subject being administered a placebo.
